(19) United States
(12) Patent Application Publication
(10) Pub. No.: US 2024/0336630 A1
(43) Pub. Date: Oct. 10, 2024

(54) MAP4K1 INHIBITORS

(71) Applicant: Blueprint Medicines Corporation, Cambridge, MA (US)

(72) Inventors: Jason D. Brubaker, Cambridge, MA (US); Michael J. Burke, Cambridge, MA (US); Joshua T. Close, Cambridge, MA (US); Thomas A. Dineen, Cambridge, MA (US); Joseph L. Kim, Cambridge, MA (US); Chandrasekhar V. Miduturu, Cambridge, MA (US); Emanuele Perola, Cambridge, MA (US)

(21) Appl. No.: 18/578,926

(22) PCT Filed: Jul. 14, 2022
(86) PCT No.: PCT/US2022/073718

§ 371 Date: Jan. 12, 2024

Related U.S. Application Data
(60) Provisional application No. 63/222,341, filed on Jul. 15, 2021.

(51) Int. Cl. C07D 519/00; A61K 31/4375; A61K 31/497; A61K 31/506; A61K 39/395; C07D 401/12; C07D 471/04

(52) U.S. Cl. CPC C07D 519/00; A61K 31/4375; A61K 31/497; A61K 31/506; A61K 39/3955; C07D 401/12; C07D 471/04

(57) ABSTRACT

One embodiment of the disclosure is a compound represented by Formula I or a pharmaceutically acceptable salt thereof. The variables in Formula I are defined herein. Compounds of Formula I are MAP4K1 inhibitors, which can be used to treat a diseases or disorders in a subject that benefits from control of MAP4K1 activity.

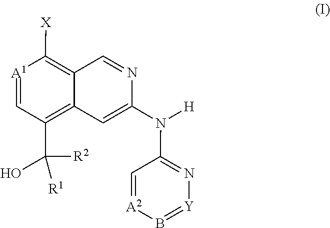

### RELATED APPLICATIONS

This application claims the benefit of U.S. Provisional Application Ser. No. 63/222,341, filed Jul. 15, 2021, the entire teachings of which are incorporated herein by reference.

### FIELD

This application is directed to MAP4K1 inhibitors and methods for their use, such as to control the activity of MAP4K1 in a subject.

### BACKGROUND

MAP4K1, also known as hematopoietic progenitor kinase 1 (HPK1), was originally cloned from hematopoietic progenitor cells (Hu, M. C., et al., Genes Dev, 1996. 10(18): p. 2251-64). MAP4K1 is of particular interest as a target, because it is predominantly expressed in hematopoietic cells such as T cells, B cells, macrophages, dendritic cells, neutrophils, and mast cells (Hu, M. C., et al, Genes Dev, 1996. 10(18): p. 2251-64; Kiefer, F., et al, EMBO J, 1996. 15(24): p. 7013-25). MAP4K1 kinase activity has been shown to be induced upon activation of T cell receptors (TCR) (Liou, J., et al., Immunity, 2000. 12(4): p. 399-408), B cell receptors (BCR) (Liou, J., et al., Immunity, 2000. 12(4): p. 399-408), transforming growth factor receptor (TGF-R) (Wang, W., et al, J Biol Chem, 1997. 272(36): p. 22771-5; Zhou, G., et al, J Biol Chem, 1999. 274(19): p. 13133-8), or Gₛ-coupled PGE₂ receptors (EP2 and EP4) (Ikegami, R, et al, J Immunol, 2001. 166(7): p. 4689-96). As such, MAP4K1 regulates diverse functions of various immune cells.

MAP4K1 is important in regulating the functions of various immune cells and it has been implicated in autoimmune diseases and anti-tumor immunity (Shui, J. W., et al, Nat Immunol, 2007. 8(1): p. 84-91; Wang, X., et al, J Biol Chem, 2012. 287(14): p. 11037-48). Those observations suggested that attenuation of MAP4K1 activity may contribute to autoimmunity in patients. Furthermore, MAP4K1 may also control anti-tumor immunity via T cell-dependent mechanisms. In the PGE2-producing Lewis lung carcinoma tumor model, the tumors developed more slowly in MAP4K1 knockout mice as compared to wild-type mice (see US 2007/0087988). In addition, it was shown that adoptive transfer of MAP4K1 deficient T cells was more effective in controlling tumor growth and metastasis than wild-type T cells (Alzabin, S., et al., Cancer Immunol Immunother, 2010. 59(3): p. 419-29). Similarly, bone marrow derived dendritic cells (BMDCs) from MAP4K1 knockout mice were more efficient to mount a T cell response to eradicate Lewis lung carcinoma as compared to wild-type BMDCs (Alzabin, S., et al., J Immunol, 2009. 182(10): p. 6187-94). Data obtained from MAP4K1 kinase dead mice demonstrated that MAP4K1 kinase activity is critical in conferring suppressive functions of MAP4K1 in a wide range of immune cells including CD4+, CD8+, DC, NK to T regulatory cells (Tregs) and inactivation of kinase domain was sufficient to elict robust anti-tumor immune responses. Liu et al., PLoS ONE 14(3):e0212670 https://doi.org/10.1371/journal.pone.0212670. Moreover, loss of MAP4K1 kinase function suppresses tumor growth in preclinical tumor models and therapeutic co-blockade of MAP4K1 kinase and PD-L1 enhances anti-tumor responses. Hernandez S. et al., Cell Reports 2018 25: p. 80-94. Recently presented results show tumor growth inhibition in a CT-26 syngeneic mouse model using a small molecule MAP4K1 inhibitor (Seungmook, L., Cancer research. AACR Journal, 2019, Abstract 4150). These data have validated MAP4K1 as a novel drug target for enhancing antitumor immunity.

Accordingly, there is a need for new compounds that modulate MAP4K1 activity for the treatment of MAP4K1-dependent diseases or disorders such as cancer, viral infection, and other diseases and disorders. Of particular importance is the need for new compounds that selectively modulate MAP4K1 activity.

### SUMMARY

Provided herein are compounds, or pharmaceutically acceptable salts thereof, and compositions which inhibit MAP4K1, thereby enhancing an immune response in a subject. For example, the IC₅₀ values for inhibition of MAP4K1 provided in Table 3 demonstrate that these compounds are potent inhibitors of MAP4K1. Also disclosed are methods of using the compounds and compositions described herein for treating cancer and viral infection.

A first embodiment of the disclosure is a compound represented by Formula I:

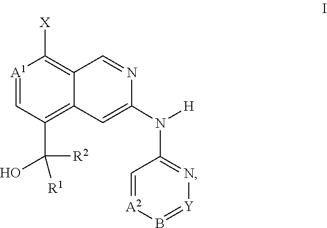

or a pharmaceutically acceptable salt thereof, wherein:
A¹ is selected from N and CH;
A² is selected from CH and N;
X is selected from C₁₋₃ alkyl, OR³, NHR⁴ and halogen;
B is selected from CR⁵ and N, Y is CR⁶, or
Y and B, taken together, form a 5 to 7-membered heterocycle or C₅₋₆ cycloalkyl, wherein said heterocycle or cycloalkyl is optionally substituted with 1-6 R⁷;
R¹ and R² are each independently selected from hydrogen, C₁₋₆ alkyl, C₁₋₆ haloalkyl, C₁₋₆ alkyl substituted with OR⁸, phenyl, C₃₋₆ cycloalkyl and 4 to 6-membered heterocycle, or R¹ and R², taken together with the atoms to which they are attached, form a C₃₋₆ cycloalkyl or 4 to 6-membered heterocycle;
R³ is selected from C₁₋₃ alkyl, C₃₋₆ cycloalkyl and 4 to 6-membered heterocycle, wherein said alkyl, cycloalkyl, and heterocycle are optionally substituted with 1-3 R⁹;
R⁴ is selected from hydrogen, C₁₋₃ alkyl, C₃₋₅ cycloalkyl and 4 to 6-membered heterocycle, wherein said alkyl, cycloalkyl, and heterocycle are optionally substituted with 1-3 R¹⁰;
R⁵ is selected from hydrogen, COOH, CN, halogen, and C₁₋₃ alkoxy;
R⁶ is selected from C₁_₅ alkyl, C₄₋₆ cycloalkyl, 3 to 6-membered heterocycle, NHR¹¹, NR¹²R¹³ and OR¹⁴, wherein said alkyl, cycloalkyl or heterocycle is optionally substituted with OH, NH₂, 1-4 halogen or R¹⁵;
each R⁷ is independently selected from C₁₋₃ alkyl, halogen and OH, wherein said alkyl is optionally substituted with 1-3 halogen, or
two R⁷ attached to the same carbon form an oxo, or
two R⁷ attached to the same carbon atom taken together with the carbon atom to which they are attached form a C₃₋₅ cycloalkyl;
R⁸ is H or C₁₋₃ alkyl.
each R⁹ is independently selected from C₁₋₃ alkyl, C₃₋₆ cycloalkyl substituted with halogen, halogen, C(O)Me, SO₂Me, C(O)NR¹⁶R¹⁷, C₁₋₃ alkoxy, and OH;
each R¹⁰ is independently selected from C₁₋₃ alkyl, C₃₋₆ cycloalkyl substituted with halogen, halogen, SO₂Me, C(O)NR¹⁶R¹⁷, C₁₋₃ alkoxy, and OH;
R¹¹ is selected from C₁₋₆ alkyl and C₃₋₆ cycloalkyl, wherein said alkyl or cycloalkyl is optionally substituted with 1-3 R¹⁸;
R¹² and R¹³ are each is independently selected from C₁₋₆ alkyl and C₃₋₆ cycloalkyl, wherein said alkyl or cycloalkyl is optionally substituted with 1-3 R¹⁸;
R¹⁴ is selected from C₁₋₆ alkyl and C₃₋₆ cycloalkyl, wherein said alkyl or cycloalkyl is optionally substituted with 1-3 R¹⁸;
R¹⁵ is OH, C₁₋₃ alkyl or C₃₋₅ cycloalkyl;
R¹⁶ and R¹⁷ are each is independently selected from C₁₋₆ alkyl and C₃₋₆ cycloalkyl, wherein said alkyl or cycloalkyl is optionally substituted with 1-3 R¹⁹;
each R¹⁸ is independently halogen; and
each R¹⁹ is independently halogen.

Another embodiment of the disclosure is a pharmaceutical composition comprising a pharmaceutically acceptable carrier or excipient and a compound disclosed herein or a pharmaceutically acceptable salt thereof.

Another embodiment of the disclosure is a method of inhibiting MAP4K1 in a subject in need thereof, comprising contacting MAP4K1 with an effective amount of the compound disclosed herein or a pharmaceutically acceptable salt thereof or a pharmaceutical composition comprising a compound disclosed herein or a pharmaceutically acceptable salt thereof.

Another embodiment of the disclosure is a method for enhancing an immune response in a subject in need thereof, comprising administering to said subject an effective amount of the compound disclosed herein, or a pharmaceutically acceptable salt thereof, or a pharmaceutical composition comprising a compound disclosed herein or a pharmaceutically acceptable salt thereof.

Another embodiment of the disclosure is a method of treating a MAP4K1-dependent disorder or disease (e.g., treating a cancer) in a subject in need thereof, comprising administering to the subject an effective amount of a compound disclosed herein or a pharmaceutically acceptable salt thereof or a pharmaceutical composition comprising the compound(s).

Another embodiment of the disclosure is the use of a compound disclosed herein or a pharmaceutically acceptable salt thereof or a pharmaceutical composition comprising the compound(s), for the preparation of a medicament for treating a MAP4K1-dependent disorder or disease (e.g., treating a cancer) in a subject in need thereof.

Another embodiment of the disclosure is a compound disclosed herein or a pharmaceutically acceptable salt thereof or a pharmaceutical composition comprising the compound(s), for use in treating a MAP4K1-dependent disorder or disease (e.g., treating a cancer) in a subject in need thereof.

### DETAILED DESCRIPTION

The disclosed compounds or pharmaceutically acceptable salts thereof are MAP4K1 inhibitors, which can be used for treating a MAP4K1-dependent disorder or disease. Such diseases or disorders include cancer and viral infection.

### Compound Embodiments

Example embodiments include:

First embodiment: a compound represented by Formula I:

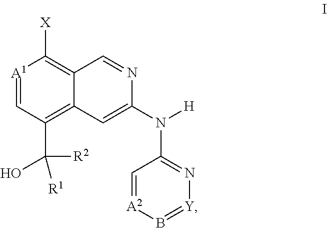

or a pharmaceutically acceptable salt thereof. The variables in Formula I are described in the summary above.

Second embodiment: a compound represented by Formula II:

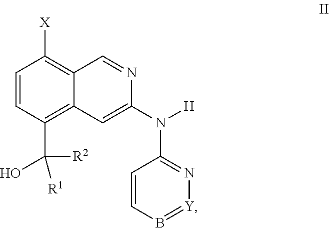

or a pharmaceutically acceptable salt thereof. The variables in Formula II are described above in the first embodiment.

Third embodiment: a compound represented by Formula III:

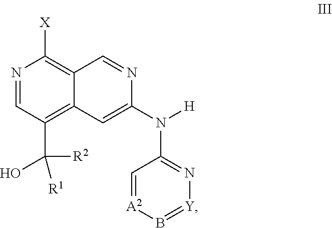

or a pharmaceutically acceptable salt thereof. The variables in Formula III are described above in the first embodiment.

Fourth embodiment: a compound represented by Formula IV(A) or IV(B):

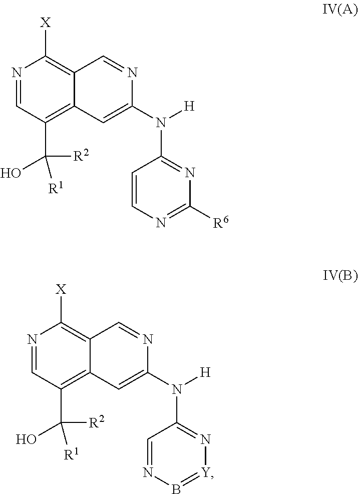

or a pharmaceutically acceptable salt thereof. The variables in Formulae IV(A) and IV(B) are described above in the first embodiment.

Fifth embodiment: a compound represented by Formula V:

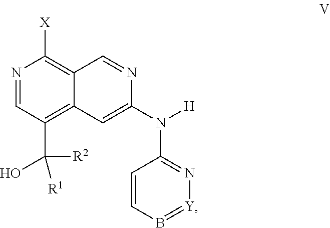

or a pharmaceutically acceptable salt thereof, wherein Y and B, taken together, form a 5 to 7-membered heterocycle or C₅₋₆ cycloalkyl, and said heterocycle or cycloalkyl is optionally substituted with 1-6 R⁷. The variables in Formula V are described above in the first embodiment.

Sixth embodiment: a compound represented by Formula VI:

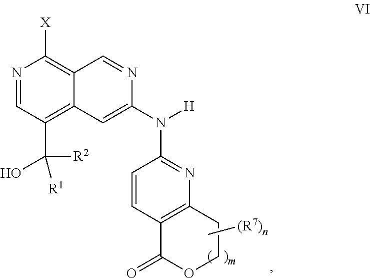

or a pharmaceutically acceptable salt thereof, wherein: each R⁷ is independently selected from C₁₋₃ alkyl, halogen and OH, wherein said alkyl is optionally substituted with 1-3 halogen, or two R⁷ attached to the same carbon atom taken together with the carbon atom to which they are attached form a C₃₋₅ cycloalkyl; n is 0, 1, 2, 3, 4, 5 or 6; and m is 0, 1 or 2. The remainder of the variables in Formula VI are described above in the first embodiment.

Seventh embodiment: a compound represented by Formula VII:

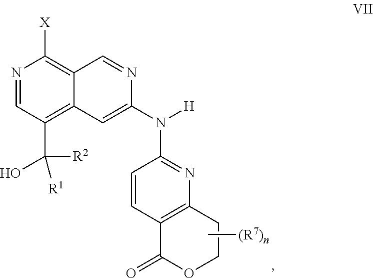

or a pharmaceutically acceptable salt thereof, wherein n is 0, 1, 2, 3 or 4. The remainder of the variables in Formula VII are described above in the first and/or sixth embodiment.

Eighth embodiment: a compound represented by Formulae I, II, III, IV(A), IV(B), V, VI or VII, or a pharmaceutically acceptable salt thereof, wherein X is NHR⁴, and R⁴ is CH₃ or cyclopropyl. The remainder of the variables in Formulae I, II, III, IV(A), IV(B), V, VI and VII are described above in the first, fifth, sixth and/or seventh embodiments.

Ninth embodiment: a compound represented by Formulae I, II, III, IV(A), IV(B), V, VI or VII, or a pharmaceutically acceptable salt thereof, wherein X is OR³. The remainder of the variables in Formulae I, II, III, IV(A), IV(B), V, VI and VII are described above in the first, fifth, sixth and/or seventh embodiments.

Tenth embodiment: a compound represented by Formulae I, II, III, IV(A), IV(B), V, VI or VII, or a pharmaceutically acceptable salt thereof, wherein: R¹ and R² are each independently selected from hydrogen, C₁₋₂ alkyl, C₁₋₂ haloalkyl, C₁₋₃ alkyl substituted with OR⁸, phenyl and C₃₋₄ cycloalkyl, or R¹ and R², taken together with the atoms to which they are attached, form a 4 to 6-membered heterocycle; and R⁸ is C₁₋₂ alkyl. The remainder of the variables in Formulae I, II, III, IV(A), IV(B), V, VI and VII are described above in the first, fifth, sixth, seventh, eighth and/or ninth embodiments.

Eleventh embodiment: a compound represented by Formulae I, II, III, IV(A), IV(B), V, VI or VII, or a pharmaceutically acceptable salt thereof, wherein: R¹ and R² are each independently selected from hydrogen, CH₃, CH₂CH₃, CH₂OCH₃, CHF₂, CF₃, cyclobutyl, cyclopropyl and phenyl, or R¹ and R², taken together with the atoms to which they are attached, form tetrahydropyran. The remainder of the variables in Formulae I, II, III, IV(A), IV(B), V, VI and VII are described above in the first, fifth, sixth, seventh, eighth and/or ninth embodiments.

Twelfth embodiment: a compound represented by Formulae I, II, III, IV(A), IV(B), V, VI or VII, or a pharmaceutically acceptable salt thereof, R¹ and R² are each independently selected from hydrogen, CH₃, CH₂CH₃, CH₂OCH₃, CHF₂, CF₃, cyclobutyl, cyclopropyl and phenyl, or

R¹ and R², taken together with the atoms to which they are attached, form

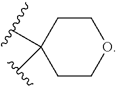 [SMILES: *C1(*)CCOCC1]

The remainder of the variables in Formulae I, II, III, IV(A), IV(B), V, VI and VII are described above in the first, fifth, sixth, seventh, eighth and/or ninth embodiments.

Thirteenth embodiment: a compound represented by Formulae I, II, III, IV(A), IV(B), V, VI or VII, or a pharmaceutically acceptable salt thereof, wherein: R³ is selected from C₁₋₃ alkyl, C₃₋₄ cycloalkyl and 4-membered heterocycle; wherein said alkyl, cycloalkyl, and heterocycle are optionally substituted with 1-3 R⁹; and each R⁹ is independently selected from C₁₋₃ alkyl, halogen, C(O)Me and SO₂Me. The remainder of the variables in Formulae I, II, III, IV(A), IV(B), V, VI and VII are described above in the first, fifth, sixth, seventh, ninth, tenth, eleventh and/or twelfth embodiments.

Fourteenth embodiment: a compound represented by Formulae I, II, III, IV(A), IV(B), V, VI or VII, or a pharmaceutically acceptable salt thereof, wherein: R³ is 4-membered heterocycle containing nitrogen; R⁹ is C(O)Me; and a ring nitrogen of the 4-membered heterocycle is bonded to —C(O)Me. The remainder of the variables in Formulae I, II, III, IV(A), IV(B), V, VI and VII are described above in the first, fifth, sixth, seventh, ninth, tenth, eleventh and/or twelfth embodiments.

Fifteenth embodiment: a compound represented by Formulae I, II, III, IV(A), IV(B), V, VI or VII, or a pharmaceutically acceptable salt thereof, wherein: R³ is selected from CH₃, CH₂CH₃, CH₂CH₂CH₃, cyclopropyl, cyclobutyl, azetidinyl, each optionally substituted with 1-3 R⁹; and each R⁹ is independently selected from CH₃, F, C(O)Me and SO₂Me. The remainder of the variables in Formulae I, II, III, IV(A), IV(B), V, VI and VII are described above in the first, fifth, sixth, seventh, ninth, tenth, eleventh and/or twelfth embodiments.

Sixteenth embodiment: a compound represented by Formulae I, II, III, IV(A), IV(B), V, VI or VII, or a pharmaceutically acceptable salt thereof, wherein R³ is selected from CH₃, CH₂CH₃, CH₂CHF₂, CH₂CF₃, CH₂CH₂CH₃, cyclopropyl:

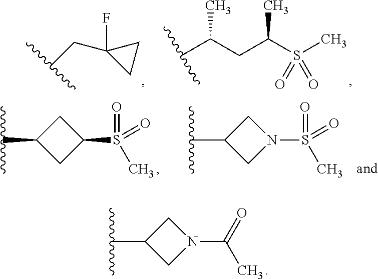

The remainder of the variables in Formulae I, II, III, IV(A), IV(B), V, VI and VII are described above in the first, fifth, sixth, seventh, ninth, tenth, eleventh and/or twelfth embodiments.

Seventeenth embodiment: a compound represented by Formulae I, II, III or IV(B), or a pharmaceutically acceptable salt thereof, wherein R⁵ is CN. The remainder of the variables in Formulae I, II, III and IV(B) are describe above in the first, eighth, ninth, tenth, eleventh, twelfth, thirteenth, fourteenth, fifteenth and/or sixteenth embodiments.

Eighteenth embodiment: a compound represented by Formulae I, II, III, IV(A) or IV(B), or a pharmaceutically acceptable salt thereof, wherein: R⁶ is selected from C₁₋₄ alkyl, and 4 to 5-membered heterocycle, wherein said alkyl or heterocycle is optionally substituted with 1-4 halogen or R¹⁵; and R¹⁵ is C₁₋₂ alkyl. The remainder of the variables in Formulae I, II, III, IV(A) and IV(B) are describe above in the first, eighth, ninth, tenth, eleventh, twelfth, thirteenth, fourteenth, fifteenth, sixteenth and/or seventeenth embodiments.

Nineteenth embodiment: a compound represented by Formulae I, II, III, IV(A) or IV(B), or a pharmaceutically acceptable salt thereof, wherein R⁶ is selected from CH(CH₃)₂, CF(CH₃)₂, C(CH₃)₃,

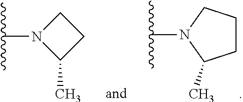

The remainder of the variables in Formulae I, II, III, IV(A) and IV(B) are describe above in the first, eighth, ninth, tenth, eleventh, twelfth, thirteenth, fourteenth, fifteenth, sixteenth and/or seventeenth embodiments.

Twentieth embodiment: a compound represented by Formulae I, II, III, IV(B), V, VI or VII, or a pharmaceutically acceptable salt thereof, wherein: each R⁷ is independently CH₃, or two R⁷ attached to the same carbon form an oxo, or two R⁷ attached to the same carbon atom taken together with the carbon atom to which they are attached form cyclopropyl. The remainder of the variables in Formulae I, II, III, IV(B), V, VI and VII are described above in the first, fifth, sixth, seventh, eighth, ninth, tenth, eleventh, twelfth, thirteenth, fourteenth, fifteenth and/or sixteenth embodiments.

Twenty first embodiment: a compound represented by Formulae I, II, III, IV(B) or V, or a pharmaceutically acceptable salt thereof, wherein Y and B, taken together, form:

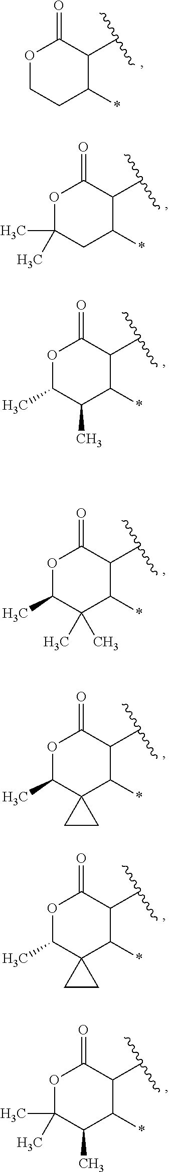

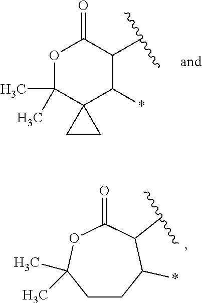

wherein [symbol] represents a bond to A², and [symbol] represents a bond to N. The remainder of the variables in Formulae I, II, III, IV(B), V, VI and VII are describe above in the first, eighth, ninth, tenth, eleventh, twelfth, thirteenth, fourteenth, fifteenth and/or sixteenth embodiments.

In one embodiment, the Compounds 71-74 in Table 4, and pharmaceutically acceptable salts thereof, are excluded from the disclosure.

The disclosure also includes the compounds depicted in Table 1 and prepared in the Exemplification, both the neutral form and pharmaceutically acceptable salts thereof. The synthetic protocol used to prepare compounds in Table 1 is listed in the last column of Table 1 and full details for each synthetic protocol are described in Scheme 1 in the General Synthetic Methods and Intermediates section.

TABLE 1

| | | | | Synthetic |
|---|---|---|---|---|
| Cpd. | | LCMS | | Protocol and |
| No. | Structure | (M + 1) | NMR | Intermediates |
| 1 | 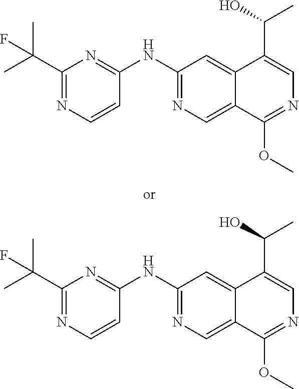 | 358 | ¹H NMR (400 MHz, CD₃OD): δ ppm 9.36 (d, J = 0.8 Hz, 1H), 8.83 (s, 1H), 8.42 (d, J = 6.0 Hz, 1H), 8.20 (s, 1H), 7.23 (d, J = 6.0 Hz, 1H), 5.40 (q, J = 6.4 Hz, 1H), 4.15 (s, 3H), 1.88 (s, 3H), 1.82 (s, 3H), 1.64 (d, J = 6.4 Hz, 3H). | Scheme 1, Intermediate 14 and Intermediate 20, 1st eluting isomer by chiral SFC Column: Daicel Chiralcel OJ-H (250 mm*30 mm, 5 um); Mobile Phase: [0.1% NH3H2O MEOH]; B %: 20%-20%) |
| 2 | 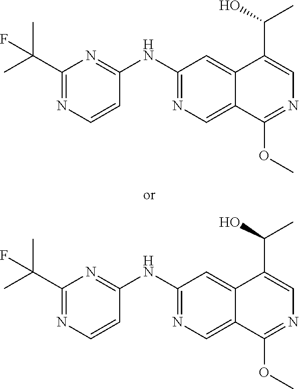 | 358 | ¹H NMR (400 MHz, CD₃OD): δ ppm 9.34 (d, J = 0.8 Hz, 1H), 8.82 (s, 1H), 8.41 (d, J = 6.0 Hz, 1H), 8.19 (s, 1H), 7.22 (d, J = 6.0 Hz, 1H), 5.39 (q, J = 6.4 Hz, 1H), 4.14 (s, 3H), 1.88 (s, 3H), 1.82 (s, 3H), 1.64 (d, J = 6.4 Hz, 3H). | Scheme 1, Intermediate 14 and Intermediate 20, 2nd eluting isomer by chiral SFC Column: Daicel Chiralcel OJ-H 250 mm*30 mm, 5um); Mobile Phase: [0.1% NH3H2O MEOH]; B % 20%-20%) |
| 3 | 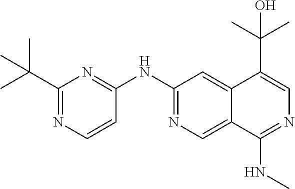 [SMILES: CNc1ncc(C(C)(C)O)c2cc(Nc3ccnc(C(C)(C)C)n3)ncc12] | 367 | ¹H NMR (400 MHz, CD3OD): δ ppm 9.21 (s, 1H), 8.58 (s, 1H), 8.31 (d, J = 6.0 Hz, 1H), 8.00 (s, 1H), 7.47 (d, J = 6.0 Hz, 1H), 3.05 (s, 3H), 1.75 (s, 6H), 1.43 (s, 9H). | Scheme 1, Intermediate 21 and 2-(tert- butyl) pyrimidin-4- amine |
| 4 | 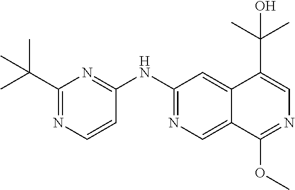 [SMILES: COc1ncc(C(C)(C)O)c2cc(Nc3ccnc(C(C)(C)C)n3)ncc12] | 368 | ¹H NMR (400 MHz, CD3OD): δ ppm 9.36 (s, 1H), 8.76 (s, 1H), 8.31 (d, J = 6.0 Hz, 1H), 8.14 (s, 1H), 7.40 (d, J = 6.0 Hz, 1H), 4.12 (s, 3H), 1.79 (s, 6H), 1.45 (s, 9H). | Scheme 1, Intermediate 22 and 2-(tert- butyl) pyrimidin-4- amine |
| 5 | 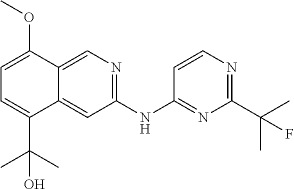 [SMILES: COc1ccc(C(C)(C)O)c2cc(Nc3ccnc(C(C)(C)F)n3)ncc12] | 371 | ¹H-NMR (400 MHz, MeOD) δ ppm 9.41 (s, 1H), 9.02 (s, 1H), 8.34 (d, 1H, J = 6.0 Hz), 7.76 (d, 1H, J = 8.0 Hz), 7.33 (d, 1H, J = 6.0 Hz), 6.84 (d, 1H, J = 8.4 Hz), 4.05 (s, 3H), 1.84 (t, 12H, J = 9.2 Hz). | Scheme 1, Intermediate 14 and Intermediate 29, Reaction Conditions: XPhosPre-catG2 Cs2CO3 Dioxane, 120° C., 12 h |
| 6 | 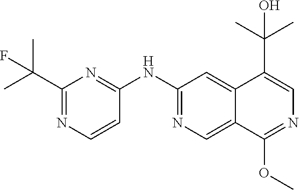 [SMILES: COc1ncc(C(C)(C)O)c2cc(Nc3ccnc(C(C)(C)F)n3)ncc12] | 372 | ¹H NMR (400 MHz, CD₃OD): δ ppm 9.35 (s, 1H), 9.05 (s, 1H), 8.37 (d, J = 6.0 Hz, 1H), 8.16 (s, 1H), 7.33 (d, J = 6.0 Hz, 1H), 4.12 (s, 3H), 1.84 (s, 3H), 1.79 (s, 9H). | Scheme 1, Intermediate 14 and Intermediate 22 |
| 7 | 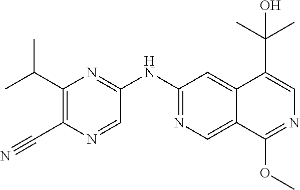 [SMILES: COc1ncc(C(C)(C)O)c2cc(Nc3cnc(C#N)c(C(C)C)n3)ncc12] | 379 | ¹H NMR (400 MHz, CD₃OD): δ ppm 9.38 (s, 1H), 9.07 (s, 1H), 8.65 (s, 1H), 8.17 (s, 1H), 4.13 (s, 3H), 3.50-3.41 (m, 1H), 1.80 (s, 6H), 1.45 (d, J = 6.8 Hz, 6H). | Scheme 1, Intermediate 15 and Intermediate 22 |
| 8 | 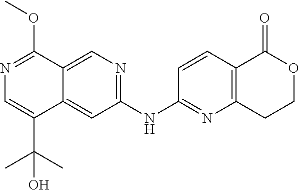 [SMILES: COc1ncc(C(C)(C)O)c2cc(Nc3ccc4c(n3)CCOC4=O)ncc12] | 381 | | Scheme 1, Intermediate 6 and Intermediate 22 |
| 9 | 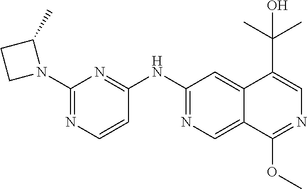 [SMILES: COc1ncc(C(C)(C)O)c2cc(Nc3ccnc(N4CC[C@@H]4C)n3)ncc12] | 381 | ¹H NMR (400 MHz, CD₃OD): δ ppm 9.32 (s, 1H), 8.83 (s, 1H), 8.12 (s, 1H), 7.96 (d, J = 5.6 Hz, 1H), 6.66 (d, J = 6.0 Hz, 1H), 4.60-4.52 (m, 1H), 4.24-4.16 (m, 1H), 4.12 (s, 3H), 4.10-4.02 (m, 1H), 2.59- 2.46 (m, 1H), 2.04-1.90 (m, 1H), 1.76 (d, J = 1.6 Hz, 6H), 1.51 (d, J = 6.4 Hz, 3H). | Scheme 1, Intermediate 18 and Intermediate 22 |
| 10 | 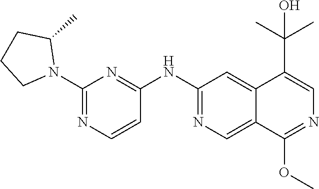 [SMILES: COc1ncc(C(C)(C)O)c2cc(Nc3ccnc(N4CCC[C@@H]4C)n3)ncc12] | 395 | ¹H NMR (400 MHz, CD₃OD): δ ppm 9.33 (s, 1H), 8.87 (s, 1H), 8.11 (s, 1H), 7.97 (d, J = 6.0 Hz, 1H), 6.59 (d, J = 5.2 Hz, 1H), 4.46-4.36 (m, 1H), 4.12 (s, 3H), 3.82-3.75 (m, 1H), 3.68-3.57 (m, 1H), 2.18- 2.07 (m, 2H), 2.05-1.96 (m, 1H), 1.77 (s, 6H), 1.24 (d, J = 6.0 Hz, 3H). | Scheme 1, Intermediate 17 and Intermediate 22 |
| 11 | 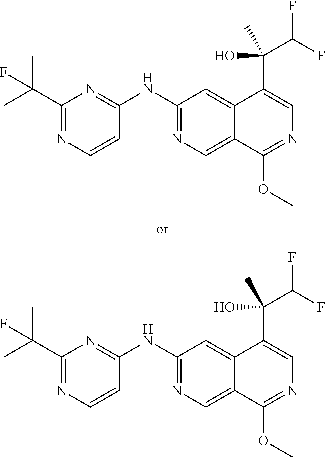 [SMILES: COc1ncc([C@@](C)(O)C(F)F)c2cc(Nc3ccnc(C(C)(C)F)n3)ncc12 ; COc1ncc([C@](C)(O)C(F)F)c2cc(Nc3ccnc(C(C)(C)F)n3)ncc12] | 408 | ¹H NMR (400 MHz, CD₃OD): δ ppm 9.37 (s, 1H), 9.17 (s, 1H), 8.37 (d, J = 4.0 Hz, 1H), 8.18 (s, 1H), 7.26 (d, J = 8.0 Hz, 1H), 6.49-6.07 (m, 1H), 4.14 (s, 3H), 1.84 (d, J = 4.0 Hz, 6H), 1.79 (s, 3H). | Scheme 1, Intermediate 14 and Intermediate 23, 1st eluting isomer from chiral SFC Column: Daicel Chiralpak AD (250 mm*30 mm, 10 um); Mobile Phase: [0.1% NH3H2O IPA]; B %: 40%-40%) |
| 12 | 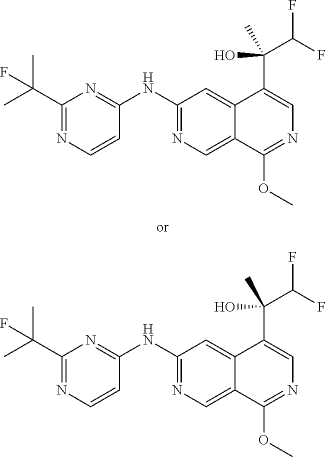 [SMILES: COc1ncc([C@@](C)(O)C(F)F)c2cc(Nc3ccnc(C(C)(C)F)n3)ncc12 ; COc1ncc([C@](C)(O)C(F)F)c2cc(Nc3ccnc(C(C)(C)F)n3)ncc12] | 408 | ¹H NMR (400 MHz, CD₃OD): δ ppm 9.37 (s, 1H), 9.17 (s, 1H), 8.37 (d, J = 4.0 Hz, 1H), 8.18 (s, 1H), 7.26 (d, J = 8.0 Hz, 1H), 6.42-6.11 (m, 1H), 4.14 (s, 3H), 1.84 (d, J = 4.0 Hz, 6H), 1.79 (s, 3H). | Scheme 1, Intermediate 14 and Intermediate 23, 2nd eluting isomer from chiral SFC Column: Daicel Chiralpak AD (250 mm*30 mm, 10 um); Mobile Phase: [0.1% NH3H2O IPA]; B %: 40%-40%) |
| 13 | 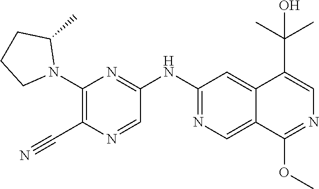 [SMILES: COc1ncc(C(C)(C)O)c2cc(Nc3cnc(C#N)c(N4CCC[C@@H]4C)n3)ncc12] | 420 | ¹H NMR (400 MHz, CD₃OD): δ ppm 9.34 (s, 1H), 8.80 (s, 1H), 8.11 (s, 1H), 8.07 (s, 1H), 4.81-4.78 (m, 1H), 4.12 (s, 3H), 4.08-3.99 (m, 1H), 3.85- 3.76 (m, 1H), 2.24-2.11 (m, 2H), 2.08-1.99 (m, 1H), 1.82- 1.77 (m, 1H), 1.75 (s, 6H), 1.29 (d, J = 6.4 Hz, 3H). | Scheme 1, Intermediate 16 and Intermediate 22 |
| 14 | 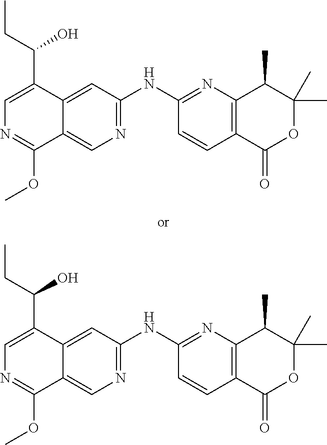 | 423 | ¹H NMR (400 MHz, 6d- DMSO): δ ppm 10.72 (s, 1H), 9.30 (s, 1H), 8.93 (s, 1H), 8.07 (d, J = 8.0 Hz, 2H), 7.32 (d, J = 8.8 Hz, 1H), 5.32 (d, J = 4.0 Hz, 1H), 4.95-4.91 (m, 1H), 4.06 (s, 3H), 3.17-3.15 (m, 1H), 1.90-1.70 (m, 2H), 1.44- 1.35 (m, 9H), 9.25 (t, J = 7.2 Hz, 3H). | Scheme 1, Intermediate 3 and Intermediate 24, 1st eluting isomer from chiral SFC Column: Daicel Chiralpak AD (250 mm*50 mm, 10 um); Mobile Phase: [0.1% NH3H2O MEOH]; B %: 30%-30%) |
| 15 | 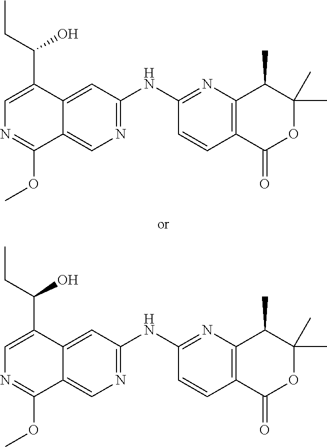 | 423 | ¹H NMR (400 MHz, 6d- DMSO): δ ppm 10.72 (s, 1H), 9.30 (s, 1H), 8.93 (s, 1H), 8.07 (d, J = 8.0 Hz, 2H), 7.32 (d, J = 8.8 Hz, 1H), 5.32 (d, J = 4.0 Hz, 1H), 4.95 (m, 1H), 4.06 (s, 3H), 3.13-3.11 (m, 1H), 1.90-1.79 (m, 2H), 1.44-1.35 (m, 9H), 0.92 (t, J = 7.2 Hz, 3H). | Scheme 1, Intermediate 3 and Intermediate 24, 2nd eluting isomer from chiral SFC Column: Daicel Chiralpak AD (250 mm*50 mm, 10 um); Mobile Phase: [0.1% NH3H2O MEOH]; B % 30%-30%) |
| 16 | 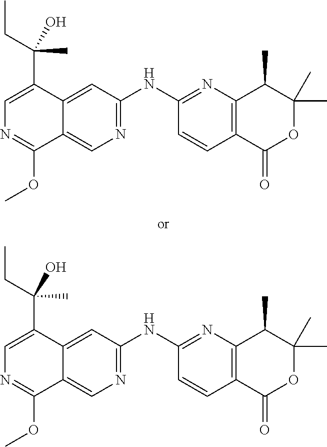 | 437 | ¹H-NMR (400 MHz, CD₃OD): δ ppm 9.23 (d, J = 5.2 Hz, 2H), 8.07 (s, 1H), 8.01 (d, J = 8.8 Hz, 1H), 7.13 (d, J = 8.8 Hz, 1H), 4.03 (s, 3H), 2.96 (d, J = 7.2 Hz, 1H), 2.20-2.04 (m, 2H), 1.69 (s, 3H), 1.42 (s, 3H), 1.35 (s, 3H), 1.30 (d, J = 7.2 Hz, 3H), 0.72 (d, J = 7.6 Hz, 3H) | Scheme 1, Intermediate 3 and Intermediate 25, 1st eluting isomer from chiral SFC Column: Daicel Chiralpak OD (250 mm*30 mm, 10 um); Mobile Phase: [0.1% NH3H2O ETOH]; B %: 35%-35%) |
| 17 | 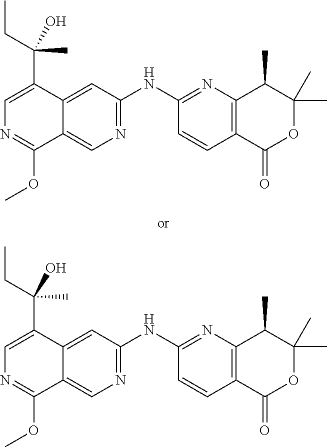 | 437 | ¹H-NMR (400 MHz, CD₃OD): δ ppm 9.28 (s, 1H), 9.21 (s, 1H), 8.06 (s, 1H), 8.01 (d, J = 8.8 Hz, 1H), 7.12 (d, J = 8.8 Hz, 1H), 4.03 (s, 3H), 3.03- 2.95 (m, 1H), 2.21-2.04 (m, 2H), 1.68 (s, 3H), 1.42 (s, 3H), 1.36 (s, 3H), 1.29 (d, J = 7.2 Hz, 3H), 0.75-0.68 (m, 3H) | Scheme 1, Intermediate 3 and Intermediate 25, 2nd eluting isomer from chiral SFC Column: Daicel Chiralpak OD (250 mm*30 mm, 10 um); Mobile Phase: [0.1% NH3H2O ETOH]; B %: 35%-35%) |
| 18 | 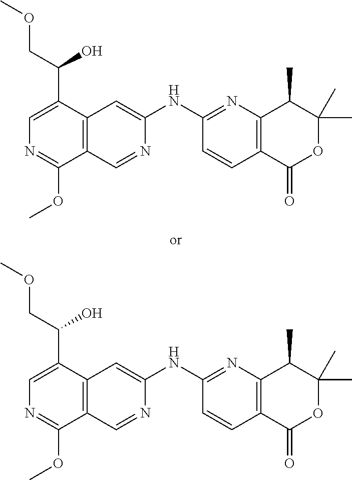 | 439 | ¹H NMR (400 MHz, CD3OD): δ ppm 9.32 (s, 1H), 9.00 (s, 1H), 8.16 (s, 1H), 8.12 (d, J = 8.4 Hz, 1H), 7.21 (d, J = 8.4 Hz, 1H), 5.35-5.30 (m, 1H), 4.13 (s, 3H), 3.78-3.75 (m, 1H), 3.73-3.65 (m, 1H), 3.43 (s, 3H), 3.20-3.15 (m, 1H), 1.55- 1.40 (m, 9H). | Scheme 1, Intermediate 3 and Intermediate 26, 1st eluting isomer from chiral SFC Column: Daicel Chiralpak IG (250 mm*50 mm, 10 um); Mobile Phase: [0.1% NH3H2O MEOH]; B %: 50%-50%) |
| 19 | 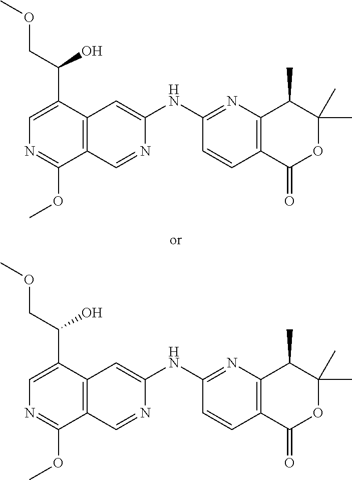 | 439 | ¹H NMR (400 MHz, CD3OD): δ ppm 9.32 (s, 1H), 8.94 (s, 1H), 8.16 (s, 1H), 8.12 (d, J = 8.4 Hz, 1H), 7.21 (d, J = 8.4 Hz, 1H), 5.38-5.30 (m, 1H), 4.13 (s, 3H), 3.78-3.75 (m, 1H), 3.70-3.63 (m, 1H), 3.41 (s, 3H), 3.25-3.15 (m, 1H), 1.55- 1.40 (m, 9H). | Scheme 1, Intermediate 3 and Intermediate 26, 2nd eluting isomer from chiral SFC Column: Daicel Chiralpak IG (250 mm*50 mm, 10 um); Mobile Phase: [0.1% NH3H2O MEOH]; B %: 50%-50%) |
| 20 | 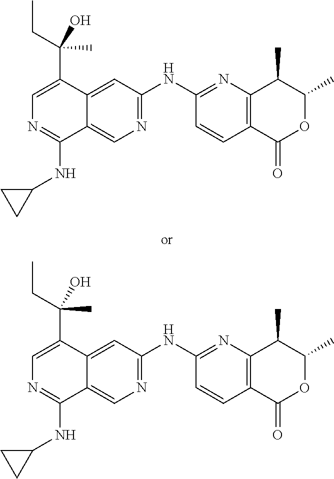 [SMILES: CC[C@@](C)(O)c1cnc(NC2CC2)c2cnc(Nc3ccc4c(n3)[C@@H](C)[C@H](C)OC4=O)cc12 ; CC[C@](C)(O)c1cnc(NC2CC2)c2cnc(Nc3ccc4c(n3)[C@@H](C)[C@H](C)OC4=O)cc12] | 448 | ¹H NMR (400 MHz, CD₃OD): δ ppm 9.22 (s, 1H), 9.05 (s, 1H), 8.12 (d, J = 8.8 Hz, 1H), 8.05 (s, 1H), 7.33 (d, J = 8.8 Hz, 1H), 4.67-4.57 (m, 1H), 3.06-2.96 (m, 1H), 2.87-2.78 (m, 1H), 2.21-2.13 (m, 2H), 1.75 (s, 3H), 1.50 (d, J = 3.8 Hz, 3H), 1.46 (d, J = 6.4 Hz, 3H), 0.92-0.86 (m, 2H), 0.82 (t, J = 7.4 Hz, 3H), 0.69-0.64 (m, 2H). | Scheme 1, Intermediate 1 and Intermediate 27 |
| 21 | 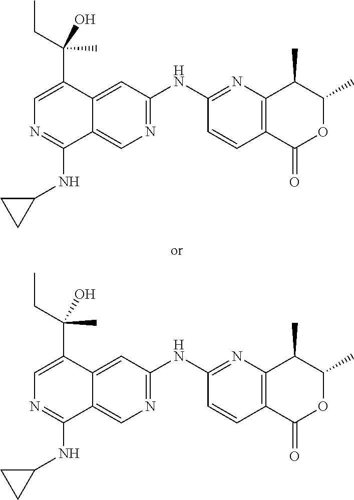 [SMILES: CC[C@@](C)(O)c1cnc(NC2CC2)c2cnc(Nc3ccc4c(n3)[C@@H](C)[C@H](C)OC4=O)cc12 ; CC[C@](C)(O)c1cnc(NC2CC2)c2cnc(Nc3ccc4c(n3)[C@@H](C)[C@H](C)OC4=O)cc12] | 448 | ¹H NMR (400 MHz, CD₃OD): δ ppm 9.21 (s, 1H), 9.08 (s, 1H), 8.12 (d, J = 8.8 Hz, 1H), 8.04 (s, 1H), 7.31 (d, J = 8.8 Hz, 1H), 4.68-4.56 (m, 1H), 3.09-2.99 (m, 1H), 2.87-2.78 (m, 1H), 2.32-2.09 (m, 2H), 1.75 (s, 3H), 1.48 (t, J = 6.8 Hz, 3H), 0.92-0.85 (m, 2H), 0.81 (t, J = 7.4 Hz, 3H), 0.70- 0.63 (m, 2H). | Scheme 1, Intermediate 1 and Intermediate 27 |
| 22 | 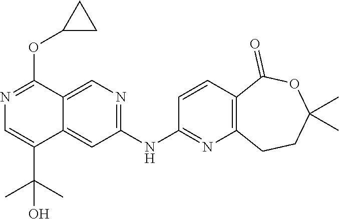 [SMILES: CC1(C)CCc2nc(Nc3cc4c(C(C)(C)O)cnc(OC5CC5)c4cn3)ccc2C(=O)O1] | 449 | ¹H-NMR (400 MHz, CD₃OD): δ ppm 9.36 (s, 1H), 9.13 (s, 1H), 8.08 (s, 1H), 7.97 (d, J = 8.4 Hz, 1H), 7.07 (d, J = 8.8 Hz, 1H), 4.36-4.33 (m, 1H), 3.16-3.15 (m, 2H), 2.22-2.18 (m, 2H), 1.74 (s, 6H), 1.32 (s, 6H), 0.80-0.76 (m, 4H). | Scheme 1, Intermediate 11 and Intermediate 30 |
| 23 | 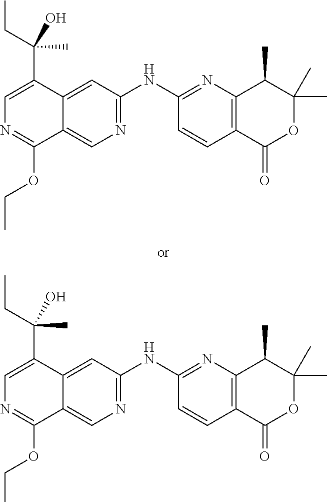 | 451 | ¹H-NMR (400 MHz, CD₃OD): 9.37 (s, 1H), 9.33 (s, 1H), 8.17 (s, 1H), 8.13 (d, J = 8.8 Hz, 1H), 7.25 (d, J = 8.4 Hz, 1H), 4.63-4.53 (m, 2H), 3.12-3.03 (m, 1H), 2.30-2.14 (m, 2H), 1.80 (s, 3H), 1.58-1.50 (m, 6H), 1.47 (s, 3H), 1.45-1.39 (m, 3H), 0.84 (t, J = 7.2 Hz, 3H). | Scheme 1, Intermediate 3 and Intermediate 31 |
| 24 | 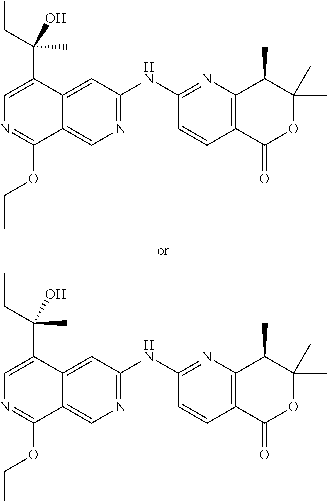 | 451 | ¹H-NMR (400 MHz, CD₃OD): 9.40 (s, 1H), 9.37 (s, 1H), 8.15 (s, 1H), 8.13 (d, J = 8.4 Hz, 1H), 7.24 (d, J = 8.8 Hz, 1H), 4.62-4.55 (m, 2H), 3.17-3.07 (m, 1H), 2.36-2.16 (m, 2H), 1.80 (s, 3H), 1.57-1.51 (m, 6H), 1.48 (s, 3H), 1.41 (d, J = 7.2 Hz, 3H), 0.83 (t, J = 7.2 Hz, 3H). | Scheme 1, Intermediate 3 and Intermediate 32 |
| 25 | 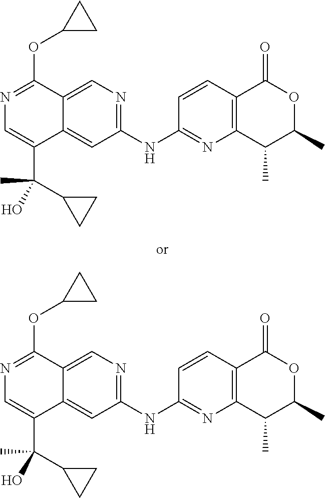 | 461 | ¹H NMR (400 MHz, CD₃OD): δ ppm 9.30 (s, 1H), 9.24 (s, 1H), 8.36 (s, 1H), 8.12 (d, J = 8.8 Hz, 1H), 7.30 (d, J = 8.8 Hz, 1H), 4.64-4.55 (m, 1H), 4.48-4.42 (m, 1H), 3.11-3.02 (m, 1H), 1.75 (s, 3H), 1.64- 1.57 (m, 1H), 1.51-1.45 (m, 6H), 0.93-0.84 (m, 4H), 0.71- 0.62 (m, 1H), 0.60-0.48 (m, 3H). | Scheme 1, Intermediate 1 and Intermediate 33, 1st eluting isomer from chiral SFC Column: Daicel Chiralpak OJ- H (250 mm × 30 mm, 5 um); Mobile Phase: [0.1% NH3H2O MEOH]; B %: 30%-30%) |
| 26 | 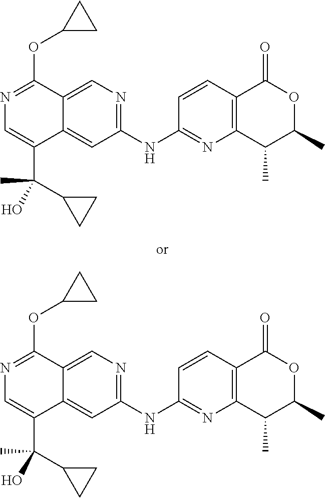 | 461 | ¹H NMR (400 MHz, CD₃OD): δ ppm 9.27 (s, 1H), 9.26 (s, 1H), 8.34 (s, 1H), 8.14 (d, J = 8.8 Hz, 1H), 7.34 (d, J = 8.4 Hz, 1H), 4.65-4.56 (m, 1H), 4.50-4.42 (m, 1H), 3.08-2.97 (m, 1H), 1.72 (s, 3H), 1.68- 1.58 (m, 1H), 1.54-1.45 (m, 6H), 0.94-0.87 (m, 4H), 0.69- 0.62 (m, 1H), 0.60-0.49 (m, 3H). | Scheme 1, Intermediate 1 and Intermediate 33, 2nd eluting isomer from chiral SFC Column: Daicel Chiralpak OJ- H (250 mm × 30 mm, 5 um); Mobile Phase: [0.1% NH3H2O MEOH]; B %: 30%-30%) |
| 27 | 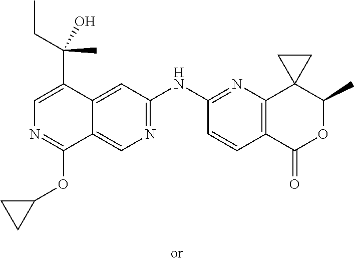 [SMILES: CC[C@](C)(O)c1cnc(OC2CC2)c2cnc(Nc3ccc4c(n3)C3(CC3)[C@@H](C)OC4=O)cc12] | 461 | ¹H NMR (400 MHz, CD3OD): δ ppm 9.25 (s, 1 H), 8.72 (s, 1 H), 8.16-8.10 (m, 2 H), 7.39 (d, J = 8.8 Hz, 1 H), 4.64-4.57 (m, 1 H), 4.49-4.42 (m, 1 H), 2.24-2.09 (m, 2 H), 1.82-1.77 (m, 1 H), 1.75 (s, 3 H), 1.58- 1.50 (m, 1 H), 1.37 (d, J = 6.4 Hz, 3 H), 1.23-1.14 (m, 2 H), 0.89 (s, 4 H), 0.84-0.79 (m, 3 H). | Scheme 1, Intermediate 8 and Intermediate 35 |
| | 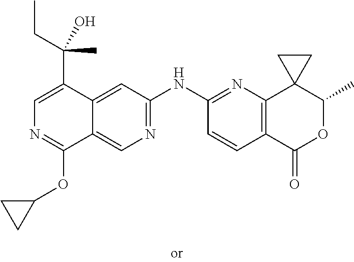 [SMILES: CC[C@](C)(O)c1cnc(OC2CC2)c2cnc(Nc3ccc4c(n3)C3(CC3)[C@H](C)OC4=O)cc12] | | | |
| | 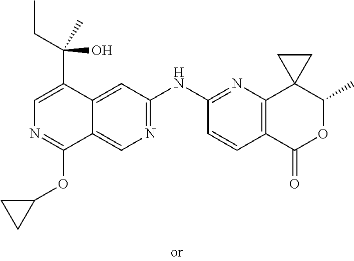 [SMILES: CC[C@@](C)(O)c1cnc(OC2CC2)c2cnc(Nc3ccc4c(n3)C3(CC3)[C@H](C)OC4=O)cc12] | | | |
| | 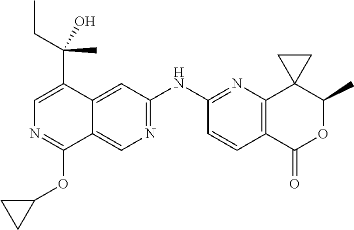 [SMILES: CC[C@](C)(O)c1cnc(OC2CC2)c2cnc(Nc3ccc4c(n3)C3(CC3)[C@@H](C)OC4=O)cc12] | | | |
| 28 | 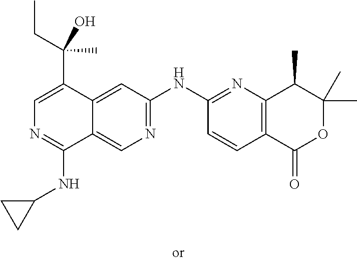 [SMILES: CC[C@@](C)(O)c1cnc(NC2CC2)c2cnc(Nc3ccc4c(n3)[C@@H](C)C(C)(C)OC4=O)cc12] | 462 | ¹H NMR (400 MHz, CD₃OD): δ ppm 9.22 (d, J = 2.0 Hz, 2H), 8.11 (d, J = 8.8 Hz, 1H), 8.05 (s, 1H), 7.24 (d, J = 8.4 Hz, 1H), 3.09-3.02 (m, 1H), 2.88-2.80 (m, 1H), 2.24-2.13 (m, 2H), 1.76 (s, 3H), 1.51 (s, 3H), 1.45 (s, 3H), 1.39 (d, J = 7.2 Hz, 3H), 0.94-0.88 (m, 2H), 0.83 (t, J = 7.4 Hz, 3H), 0.71-0.66 (m, 2H). | Scheme 1, Intermediate 3 and Intermediate 27 |
| | 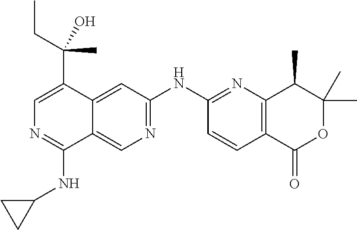 [SMILES: CC[C@](C)(O)c1cnc(NC2CC2)c2cnc(Nc3ccc4c(n3)[C@@H](C)C(C)(C)OC4=O)cc12] | | | |
| 29 | 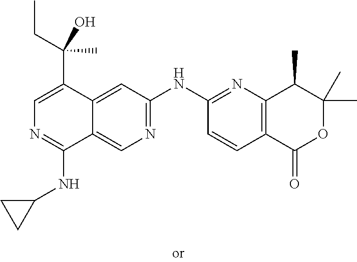 [SMILES: CC[C@@](C)(O)c1cnc(NC2CC2)c2cnc(Nc3ccc4c(n3)[C@@H](C)C(C)(C)OC4=O)cc12] | | ¹H NMR (400 MHz, CD₃OD): δ ppm 9.29 (s, 1H), 9.22 (s, 1H), 8.10 (d, J = 8.8 Hz, 1H), 8.04 (s, 1H), 7.22 (d, J = 8.4 Hz, 1H), 3.14-3.04 (m, 1H), 2.87-2.77 (m, 1H), 2.32-2.11 (m, 2H), 1.76 (s, 3H), 1.52 (s, 3H), 1.45 (s, 1H), 1.38 (d, J = 7.2 Hz, 3H), 0.95-0.86 (m, 2H), 0.82 (t, J = 7.4 Hz, 3H), 0.71-0.64 (m, 2H). | Scheme 1, Intermediate 3 and Intermediate 28 |
| | 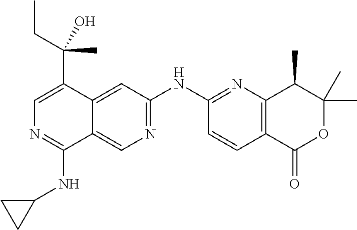 [SMILES: CC[C@](C)(O)c1cnc(NC2CC2)c2cnc(Nc3ccc4c(n3)[C@@H](C)C(C)(C)OC4=O)cc12] | | | |
| 30 | 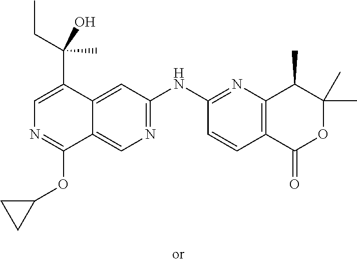 [SMILES: CC[C@@](C)(O)c1cnc(OC2CC2)c2cnc(Nc3ccc4c(n3)[C@@H](C)C(C)(C)OC4=O)cc12] | 463 | ¹H NMR (400 MHz, CDCl₃): δ ppm 9.29 (s, 1H), 9.11 (s, 1H), 8.25-8.19 (m, 2H), 7.96 (s, 1H), 7.17 (d, J = 8.8 Hz, 1H), 4.55-4.49 (m, 1H), 3.04-2.07 (m, 1H), 2.27-2.11 (m, 2H), 1.90 (s, 1H), 1.80 (s, 3H), 1.49 (d, J = 2.0 Hz, 6H), 1.44 (d, J = 6.8 Hz, 3H), 0.95-0.86 (m, 7H) | Scheme 1, Intermediate 3 and Intermediate 34 |
| | 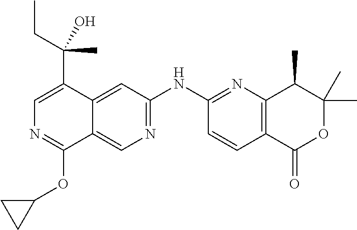 [SMILES: CC[C@](C)(O)c1cnc(OC2CC2)c2cnc(Nc3ccc4c(n3)[C@@H](C)C(C)(C)OC4=O)cc12] | | | |
| 31 | 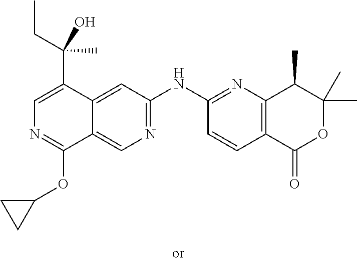 [SMILES: CC[C@@](C)(O)c1cnc(OC2CC2)c2cnc(Nc3ccc4c(n3)[C@@H](C)C(C)(C)OC4=O)cc12] | 463 | ¹H-NMR (400 MHz, CDCl₃): δ ppm 9.29 (s, 1H), 9.15 (s, 1H), 8.24-8.18 (m, 2H), 7.96 (s, 1H), 7.15 (d, J = 8.8 Hz, 1H), 4.55-4.48 (m, 1H), 3.06- 2.97 (m, 1H), 2.23-2.14 (m, 2H), 1.92 (s, 1H), 1.80 (s, 3H), 1.49 (d, J = 3.6 Hz, 6H), 1.43 (d, J = 7.2 Hz, 3H), 0.95-0.90 (m, 4H), 0.87 (t, J = 7.2 Hz, 3H) | Scheme 1, Intermediate 3 and Intermediate 35 |
| | 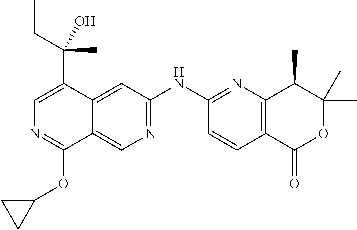 [SMILES: CC[C@](C)(O)c1cnc(OC2CC2)c2cnc(Nc3ccc4c(n3)[C@@H](C)C(C)(C)OC4=O)cc12] | | | |
| 32 | 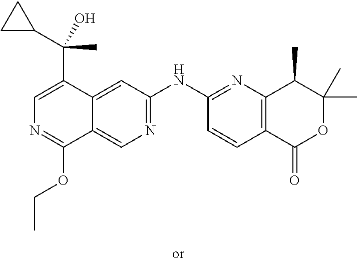 [SMILES: CCOc1ncc([C@@](C)(O)C2CC2)c2cc(Nc3ccc4c(n3)[C@@H](C)C(C)(C)OC4=O)ncc12] | 463 | ¹H-NMR (400 MHz, CD₃OD): 9.45 (s, 1H), 9.36 (s, 1H), 8.35 (s, 1H), 8.13 (d, J = 8.8 Hz, 1H), 7.27 (d, J = 8.8 Hz, 1H), 4.58 (q, J = 7.2 Hz, 2H), 3.21- 3.09 (m, 1H), 1.78 (s, 3H), 1.68-1.59 (m, 1H), 1.56-1.51 (m, 6H), 1.49 (s, 3H), 1.39 (d, J = 7.2 Hz, 3H), 0.74-0.65 (m, 1H), 0.64-0.48 (m, 3H) | Scheme 1, Intermediate 3 and Intermediate 37 |
| | 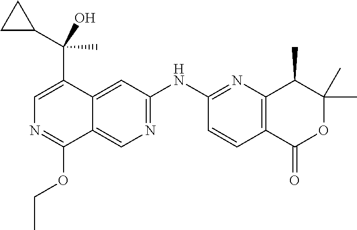 [SMILES: CCOc1ncc([C@](C)(O)C2CC2)c2cc(Nc3ccc4c(n3)[C@@H](C)C(C)(C)OC4=O)ncc12] | | | |
| 33 | 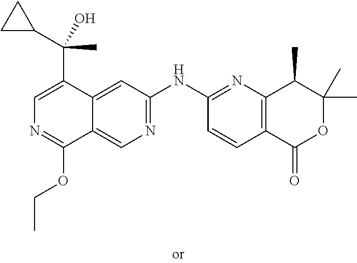 [SMILES: CCOc1ncc([C@@](C)(O)C2CC2)c2cc(Nc3ccc4c(n3)[C@@H](C)C(C)(C)OC4=O)ncc12] | 463 | ¹H-NMR (400 MHz, CD₃OD): 9.41 (s, 1H), 9.36 (s, 1H), 8.31 (s, 1H), 8.13 (d, J = 8.4 Hz, 1H), 7.29 (d, J = 8.8 Hz, 1H), 4.58 (q, J = 7.2 Hz, 2H), 3.09 (q, J = 7.2 Hz, 1H), 1.75 (s, 3H), 1.71-1.62 (m, 1H), 1.57- 1.51 (m, 6H), 1.47 (s, 3H), 1.42 (d, J = 6.8 Hz, 3H), 0.71- 0.44 (m, 4H) | Scheme 1, Intermediate 3 and Intermediate 36 |
| | 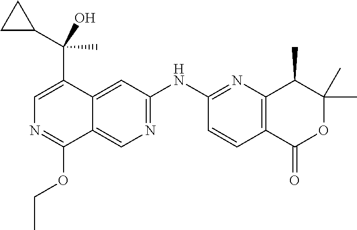 [SMILES: CCOc1ncc([C@](C)(O)C2CC2)c2cc(Nc3ccc4c(n3)[C@@H](C)C(C)(C)OC4=O)ncc12] | | | |
| 34 | 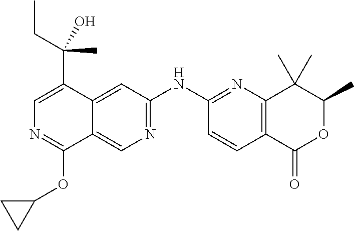 [SMILES: CC[C@](C)(O)c1cnc(OC2CC2)c2cnc(Nc3ccc4c(n3)C(C)(C)[C@@H](C)OC4=O)cc12] | 463 | ¹H NMR (400 MHz, CD3OD): δ ppm 9.26 (s, 1 H), 8.88 (s, 1 H), 8.15-8.10 (m, 2 H), 7.42 (d, J = 8.8 Hz, 1 H), 4.60-4.52 (m, 1 H), 4.51-4.38 (m, 1 H), 2.31-2.09 (m, 2 H), 1.75 (s, 3 H), 1.53 (s, 3 H), 1.45 (d, J = 6.4 Hz, 3 H), 1.31 (s, 3 H), 0.94-0.86 (m, 4 H), 0.84-0.78 (m, 3 H). | Scheme 1, Intermediate 12 and Intermediate 35 |
| 35 | 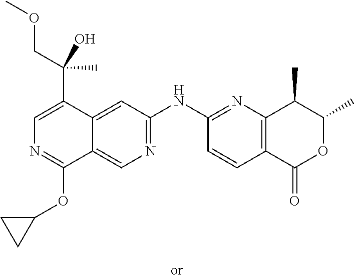 [SMILES: COC[C@@](C)(O)c1cnc(OC2CC2)c2cnc(Nc3ccc4c(n3)[C@@H](C)[C@H](C)OC4=O)cc12] | 465 | ¹H NMR (400 MHz, CDCl3): δ ppm 9.29 (s, 1H), 9.03 (s, 1H), 8.24 (d, J = 8.8 Hz, 1H), 8.15 (s, 1H), 7.81 (s, 1H), 7.23 (d, J = 8.8 Hz, 1H), 4.57-4.43 (m, 2H), 4.06 (d, J = 9.2 Hz, 1H), 3.62 (d, J = 9.2 Hz, 1H), 3.48 (s, 3H), 3.06-2.96 (m, 2H), 1.74 (s, 3H), 1.55-1.50 (m, 6H), 0.94-0.86 (m, 4H). | Scheme 1, Intermediate 1 and Intermediate 39 |
| | 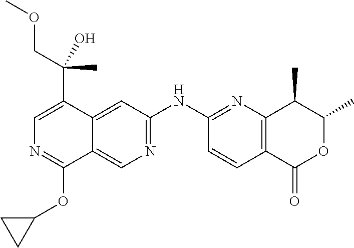 [SMILES: COC[C@](C)(O)c1cnc(OC2CC2)c2cnc(Nc3ccc4c(n3)[C@@H](C)[C@H](C)OC4=O)cc12] | | | |
| 36 | 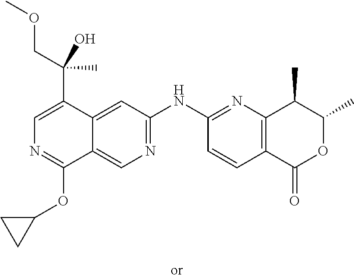 [SMILES: COC[C@@](C)(O)c1cnc(OC2CC2)c2cnc(Nc3ccc4c(n3)[C@@H](C)[C@H](C)OC4=O)cc12] | 465 | ¹H NMR (400 MHz, CDCl3): δ ppm 9.29 (s, 1H), 8.96 (s, 1H), 8.24 (d, J = 8.8 Hz, 1H), 8.15 (s, 1H), 7.83 (s, 1H), 7.25 (d, J = 8.8 Hz, 1H), 4.57-4.43 (m, 2H), 4.04 (d, J = 9.2 Hz, 1H), 3.62 (d, J = 9.2 Hz, 1H), 3.48 (s, 3H), 3.06-2.96 (m, 2H), 1.74 (s, 3H), 1.55-1.50 (m, 6H), 0.94-0.86 (m, 4H). | Scheme 1, Intermediate 1 and Intermediate 38 |
| | 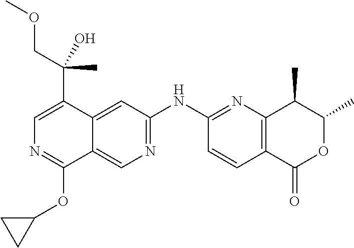 [SMILES: COC[C@](C)(O)c1cnc(OC2CC2)c2cnc(Nc3ccc4c(n3)[C@@H](C)[C@H](C)OC4=O)cc12] | | | |
| 37 | 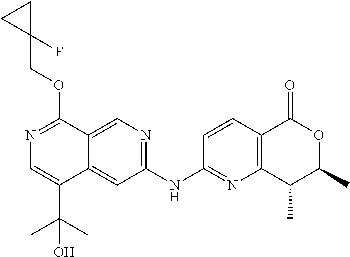 [SMILES: C[C@@H]1OC(=O)c2ccc(Nc3cc4c(C(C)(C)O)cnc(OCC5(F)CC5)c4cn3)nc2[C@H]1C] | 467 | ¹H-NMR (400 MHz, MeOD): δ ppm 9.40 (s, 1H), 9.26 (s, 1H), 8.15-8.13 (m, 2H), 7.31 (d, J = 8.8 Hz, 1H), 4.84 (s, 2H), 4.63-4.60 (m, 1H), 3.09- 3.02 (m, 1H), 1.81 (d, J = 3.6 Hz, 6H), 1.53-1.47 (m, 6H), 1.23-1.16 (m, 2H), 0.96 (d, J = 7.2 Hz, 2H) | Scheme 1, Intermediate 1 and Intermediate 40 |
| 38 | 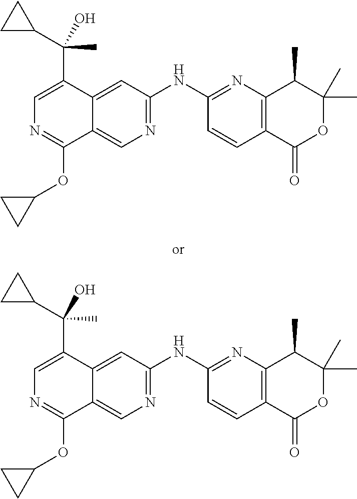 [SMILES: C[C@@H]1c2nc(Nc3cc4c([C@@](C)(O)C5CC5)cnc(OC5CC5)c4cn3)ccc2C(=O)OC1(C)C ; C[C@@H]1c2nc(Nc3cc4c([C@](C)(O)C5CC5)cnc(OC5CC5)c4cn3)ccc2C(=O)OC1(C)C] | 475 | ¹H NMR (400 MHz, CD₃OD): δ ppm 9.46 (s, 1H), 9.24 (s, 1H), 8.37 (s, 1H), 8.11 (d, J = 8.8 Hz, 1H), 7.24 (d, J = 8.8 Hz, 1H), 4.49-4.42 (m, 1H), 3.18-3.07 (m, 1H), 1.77 (s, 3H), 1.67-1.58 (m, 1H), 1.51 (s, 3H), 1.46 (s, 3H), 1.37 (d, J = 7.2 Hz, 3H), 0.93-0.85 (m, 4H), 0.72-0.64 (m, 1H), 0.62- 0.47 (m, 3H). | Scheme 1, Intermediate 3 and Intermediate 33, 1st eluting isomer from chiral SFC Column: Daicel Chiralpak IC (250 mm × 30 mm, 10 um); Mobile Phase: [0.1% NH3H2O MEOH]; B %: 60%-60%) |
| 39 | 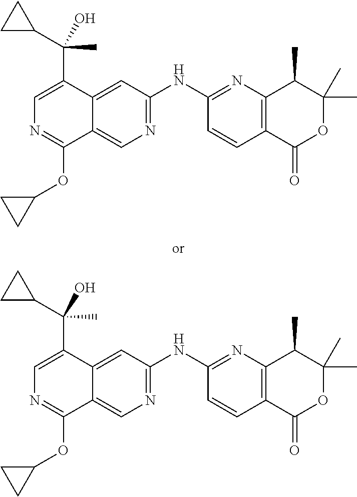 | 475 | ¹H NMR (400 MHz, CD₃OD): δ ppm 9.42 (s, 1H), 9.25 (s, 1H), 8.33 (s, 1H), 8.11 (d, J = 8.8 Hz, 1H), 7.26 (d, J = 8.4 Hz, 1H), 4.49-4.42 (m, 1H), 3.12-3.01 (m, 1H), 1.74 (s, 3H), 1.70-1.62 (m, 1H), 1.50 (s, 3H), 1.45 (s, 3H), 1.41 (d, J = 7.2 Hz, 3H), 0.93-0.85 (m, 4H), 0.68-0.62 (m, 1H), 0.61- 0.46 (m, 3H). | Scheme 1, Intermediate 3 and Intermediate 33, 2nd eluting isomer from chiral SFC Column: Daicel Chiralpak IC (250 mm × 30 mm, 10 um); Mobile Phase: [0.1% NH3H2O MEOH]; B % 60%-60%) |
| 40 | 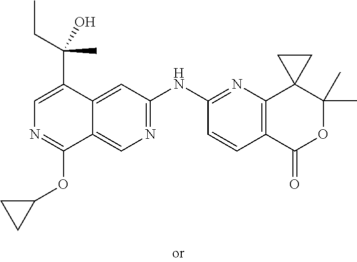 [SMILES: CC[C@](C)(O)c1cnc(OC2CC2)c2cnc(Nc3ccc4c(n3)C3(CC3)C(C)(C)OC4=O)cc12] | 475 | ¹H NMR (400 MHz, CD₃Cl): δ ppm 9.30 (s, 1H), 8.71 (s, 1H), 8.24 (d, J = 8.8 Hz, 1H), 8.15 (s, 1H), 7.83 (s, 1H), 7.21 (d, J = 8.4 Hz, 1H), 4.55-4.50 (m, 1H), 2.18-2.14 (m, 2H), 1.93 (s, 1H), 1.79 (s, 3H), 1.67-1.63 (m, 2H), 1.43 (s, 6H), 1.19- 1.18 (m, 2H), 0.94-0.88 (m, 7H). | Scheme 1, Intermediate 10 and Intermediate 35 |
| | 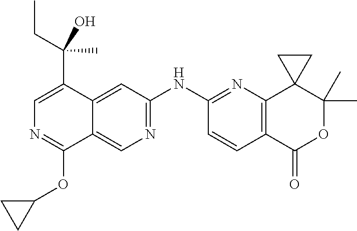 [SMILES: CC[C@@](C)(O)c1cnc(OC2CC2)c2cnc(Nc3ccc4c(n3)C3(CC3)C(C)(C)OC4=O)cc12] | | | |
| 41 | 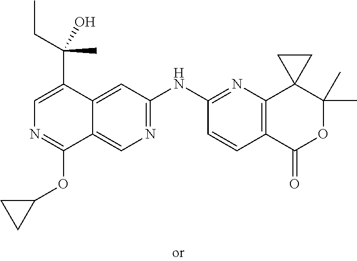 [SMILES: CC[C@](C)(O)c1cnc(OC2CC2)c2cnc(Nc3ccc4c(n3)C3(CC3)C(C)(C)OC4=O)cc12] | 475 | ¹H NMR (400 MHz, CD₃Cl): δ ppm 9.22 (s, 1H), 8.61 (s, 1H), 8.16 (d, J = 8.4 Hz, 1H), 8.06 (s, 1H), 7.66 (s, 1H), 7.12 (d, J = 8.8 Hz, 1H), 4.45-4.42 (m, 1H), 2.09-2.06 (m, 2H), 1.76 (s, 1H), 1.70 (s, 3H), 1.60-1.52 (m, 2H), 1.34 (s, 6H), 1.09 (s, 2H), 0.85-0.79 (m, 7H). | Scheme 1, Intermediate 10 and Intermediate 34 |
| | 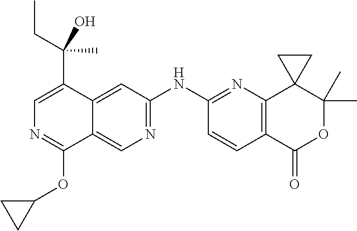 [SMILES: CC[C@@](C)(O)c1cnc(OC2CC2)c2cnc(Nc3ccc4c(n3)C3(CC3)C(C)(C)OC4=O)cc12] | | | |
| 42 | 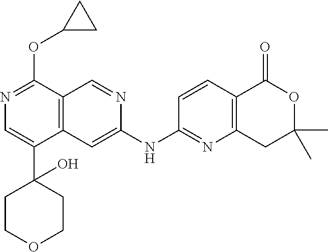 [SMILES: CC1(C)Cc2nc(Nc3cc4c(C5(O)CCOCC5)cnc(OC5CC5)c4cn3)ccc2C(=O)O1] | 477 | | Scheme 1, Intermediate 5 and Intermediate 60 |
| 43 | 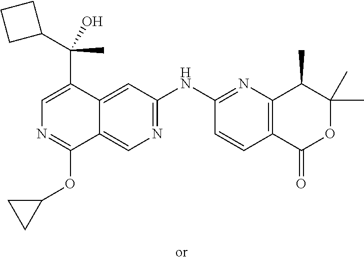 [SMILES: C[C@@H]1c2nc(Nc3cc4c([C@@](C)(O)C5CCC5)cnc(OC5CC5)c4cn3)ccc2C(=O)OC1(C)C] | 489 | ¹H NMR (400 MHz, CD₃OD): δ ppm 9.41 (s, 1H), 9.23 (s, 1H), 8.20 (s, 1H), 8.12 (d, J = 8.8 Hz, 1H), 7.22 (d, J = 8.8 Hz, 1H), 4.48-4.41 (m, 1H), 3.40-3.34 (m, 1H), 3.16-3.06 (m, 1H), 2.37-2.24 (m, 1H), 2.12-2.02 (m, 1H), 2.01-1.91 (m, 1H), 1.88-1.79 (m, 1H), 1.70 (s, 3H), 1.68-1.61 (m, 1H), 1.52 (s, 3H), 1.46 (s, 3H), 1.43 (d, J = 7.2 Hz, 3H), 0.92- 0.86 (m, 4H). | Scheme 1, Intermediate 3 and Intermediate 41 |
| | 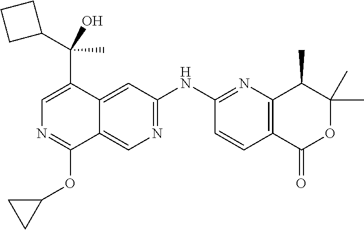 [SMILES: C[C@@H]1c2nc(Nc3cc4c([C@](C)(O)C5CCC5)cnc(OC5CC5)c4cn3)ccc2C(=O)OC1(C)C] | | | |
| 44 | 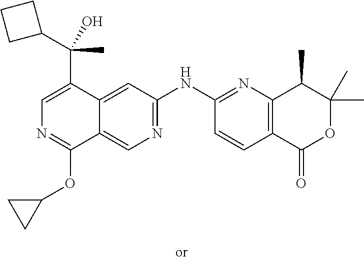 [SMILES: C[C@@H]1c2nc(Nc3cc4c([C@@](C)(O)C5CCC5)cnc(OC5CC5)c4cn3)ccc2C(=O)OC1(C)C] | 489 | ¹H NMR (400 MHz, CD₃OD): δ ppm 9.45 (s, 1H), 9.23 (s, 1H), 8.21 (s, 1H), 8.12 (d, J = 8.8 Hz, 1H), 7.23 (d, J = 8.4 Hz, 1H), 4.48-4.42 (m, 1H), 3.42-3.36 (m, 1H), 3.12-3.05 (m, 1H), 2.34-2.22 (m, 1H), 2.13-2.02 (m, 1H), 1.97-1.90 (m, 1H), 1.85-1.77 (m, 1H), 1.70 (s, 3H), 1.67-1.61 (m, 1H), 1.53 (s, 3H), 1.47 (s, 3H), 1.42 (d, J = 7.2 Hz, 3H), 0.93- 0.86 (m, 4H). | Scheme 1, Intermediate 3 and Intermediate 42 |
| | 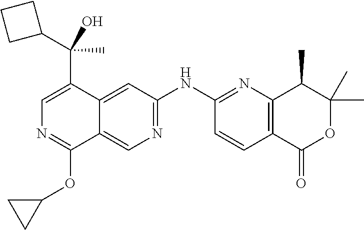 [SMILES: C[C@@H]1c2nc(Nc3cc4c([C@](C)(O)C5CCC5)cnc(OC5CC5)c4cn3)ccc2C(=O)OC1(C)C] | | | |
| 45 | 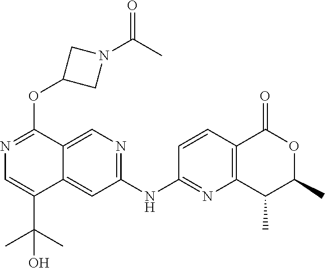 [SMILES: CC(=O)N1CC(Oc2ncc(C(C)(C)O)c3cc(Nc4ccc5c(n4)[C@@H](C)[C@H](C)OC5=O)ncc23)C1] | 492 | ¹H NMR (400 MHz, CD₃OD) δ ppm 9.42 (d, J = 3.2 Hz, 1H), 9.29 (d, J = 3.2 Hz, 1H), 8.16 (s, 1H), 8.14 (d, J = 8.8 Hz, 1H), 7.33 (d, J = 8.8 Hz, 1H), 5.63 (s, 1H), 4.77-4.69 (m, 1H), 4.62 (t, J = 6.8 Hz, 1H), 4.53-4.45 (m, 1H), 4.42- 4.33 (m, 1H), 4.19-4.09 (m, 1H), 3.07 (t, J = 6.4 Hz, 1H), 1.96 (s, 3H), 1.83 (d, J = 3.6 Hz, 6H), 1.54 (d, J = 7.2 Hz, 3H), 1.50 (d, J = 6.4 Hz, 3H). | Scheme 1, Intermediate 1 and Intermediate 43 |
| 46 | 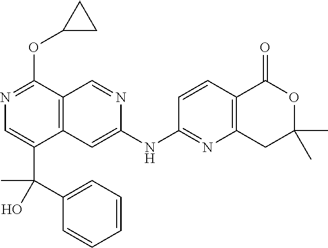 [SMILES: CC1(C)Cc2nc(Nc3cc4c(C(C)(O)c5ccccc5)cnc(OC5CC5)c4cn3)ccc2C(=O)O1] | | | Scheme 1, Intermediate 5 and Intermediate 59 |
| 47 | 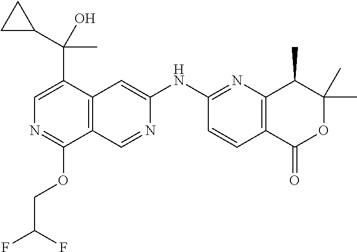 [SMILES: C[C@@H]1c2nc(Nc3cc4c(C(C)(O)C5CC5)cnc(OCC(F)F)c4cn3)ccc2C(=O)OC1(C)C] | 499 | | Scheme 1, Intermediate 3 and Intermediate 44 |
| 48 | 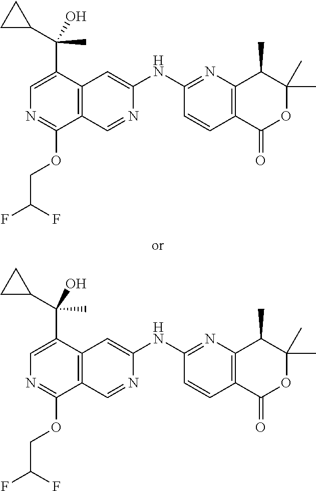 [SMILES: C[C@@H]1c2nc(Nc3cc4c([C@@](C)(O)C5CC5)cnc(OCC(F)F)c4cn3)ccc2C(=O)OC1(C)C ; C[C@@H]1c2nc(Nc3cc4c([C@](C)(O)C5CC5)cnc(OCC(F)F)c4cn3)ccc2C(=O)OC1(C)C] | 499 | | Scheme 1, Intermediate 3 and Intermediate 44, 1st eluting isomer by chiral SFC Column: ChiralPak IC-H 21 × 250 mm; Mobile Phase: 30% Methanol + 0.25% Diethylamine in CO₂) |
| 49 | 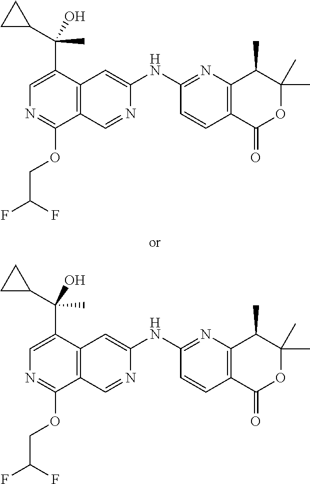 [SMILES: C[C@@H]1c2nc(Nc3cc4c([C@@](C)(O)C5CC5)cnc(OCC(F)F)c4cn3)ccc2C(=O)OC1(C)C ; C[C@@H]1c2nc(Nc3cc4c([C@](C)(O)C5CC5)cnc(OCC(F)F)c4cn3)ccc2C(=O)OC1(C)C] | 499 | | Scheme 1, Intermediate 3 and Intermediate 44, 2nd eluting isomer by chiral SFC Column: ChiralPak IC-H 21 × 250 mm; Mobile Phase: 30% Methanol + 0.25% Diethylamine in CO₂ |
| 50 | 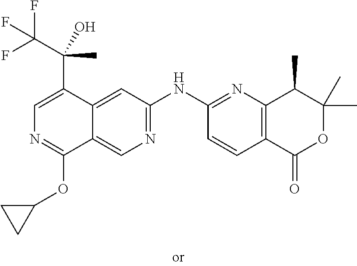 [SMILES: C[C@@H]1c2nc(Nc3cc4c([C@@](C)(O)C(F)(F)F)cnc(OC5CC5)c4cn3)ccc2C(=O)OC1(C)C] | 503 | ¹H-NMR (400 MHz, CDCl₃): δ ppm 9.29 (s, 1H), 9.18 (s, 1H), 8.29 (s, 1H), 8.21 (d, J = 8.4 Hz, 1H), 7.90 (s, 1H), 7.18 (d, J = 8.4 Hz, 1H), 4.58-4.51 (m, 1H), 3.05-2.97 (m, 1H), 2.83-2.65 (m, 1H), 2.05 (s, 3H), 1.48 (d, J = 3.6 Hz, 6H), 1.43 (d, J = 7.2 Hz, 3H), 0.97- 0.91 (m, 4H) | Scheme 1, Intermediate 3 and Intermediate 45 |
| | 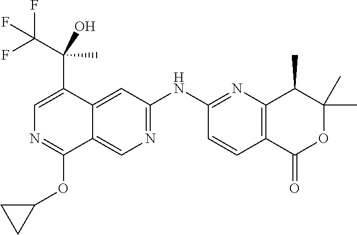 [SMILES: C[C@@H]1c2nc(Nc3cc4c([C@](C)(O)C(F)(F)F)cnc(OC5CC5)c4cn3)ccc2C(=O)OC1(C)C] | | | |
| 51 | 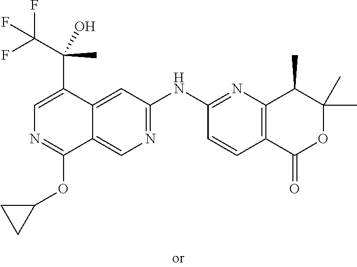 [SMILES: C[C@@H]1c2nc(Nc3cc4c([C@@](C)(O)C(F)(F)F)cnc(OC5CC5)c4cn3)ccc2C(=O)OC1(C)C] | 503 | ¹H-NMR (400 MHz, CDCl₃): δ ppm 9.30 (s, 1H), 9.13 (s, 1H), 8.29 (s, 1H), 8.22 (d, J = 8.8 Hz, 1H), 7.82 (s, 1H), 7.20 (d, J = 8.8 Hz, 1H), 4.58-4.52 (m, 1H), 3.05-2.97 (m, 1H), 2.68-2.62 (m, 1H), 2.04 (s, 3H), 1.48 (s, 6H), 1.42 (d, J = 7.2 Hz, 3H), 0.96-0.90 (m, 4H) | Scheme 1, Intermediate 3 and Intermediate 46 |
| | 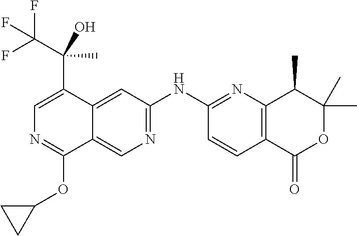 [SMILES: C[C@@H]1c2nc(Nc3cc4c([C@](C)(O)C(F)(F)F)cnc(OC5CC5)c4cn3)ccc2C(=O)OC1(C)C] | | | |
| 52 | 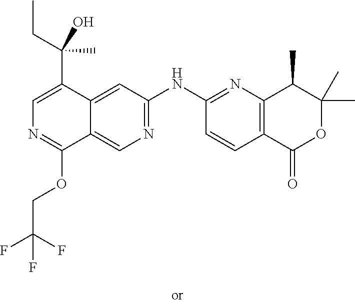 [SMILES: CC[C@@](C)(O)c1cnc(OCC(F)(F)F)c2cnc(Nc3ccc4c(n3)[C@@H](C)C(C)(C)OC4=O)cc12] | 505 | ¹H-NMR (400 MHz, CD₃OD): 9.42 (s, 1 H), 9.36 (s, 1 H), 8.22 (s, 1 H), 8.15 (d, J = 8.8 Hz, 1 H), 7.25 (d, J = 8.8 Hz, 1 H), 5.11 (q, J = 24 Hz, 2 H), 3.09 (d, J = 7.2 Hz, 1 H), 2.27- 2.19 (m, 2 H), 1.82 (s, 3 H), 1.54 (s, 3 H), 1.48 (s, 3 H), 1.40 (d, J = 7.2 Hz, 1 H), 0.85 (d, t = 14.8 Hz, 1 H). | Scheme 1, Intermediate 3 and Intermediate 47 |
| | 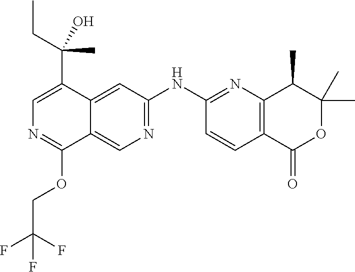 [SMILES: CC[C@](C)(O)c1cnc(OCC(F)(F)F)c2cnc(Nc3ccc4c(n3)[C@@H](C)C(C)(C)OC4=O)cc12] | | | |
| 53 | 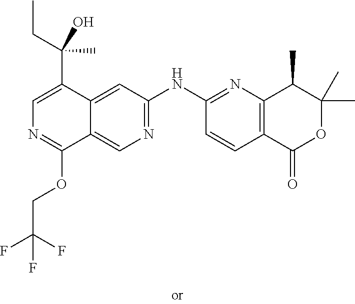 [SMILES: CC[C@@](C)(O)c1cnc(OCC(F)(F)F)c2cnc(Nc3ccc4c(n3)[C@@H](C)C(C)(C)OC4=O)cc12] | 505 | ¹H-NMR (400 MHz, CD₃OD): 9.49 (s, 1 H), 9.36 (s, 1 H), 8.21 (s, 1 H), 8.14 (d, J = 8.8 Hz, 1 H), 7.24 (d, J = 8.8 Hz, 1 H), 5.11 (q, J = 24 Hz, 2 H), 3.12 (d, J = 7.2 Hz, 1 H), 2.30- 2.20 (m, 2 H), 1.81 (s, 3 H), 1.55 (s, 3 H), 1.48 (s, 3 H), 1.41 (d, J = 7.2 Hz, 1 H), 0.84 (d, t = 14.8 Hz, 1 H). | Scheme 1, Intermediate 3 and Intermediate 48 |
| | 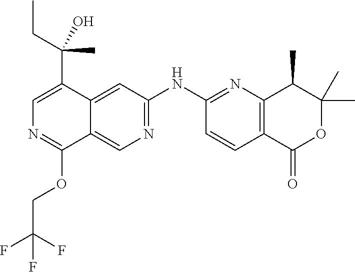 [SMILES: CC[C@](C)(O)c1cnc(OCC(F)(F)F)c2cnc(Nc3ccc4c(n3)[C@@H](C)C(C)(C)OC4=O)cc12] | | | |
| 54 | 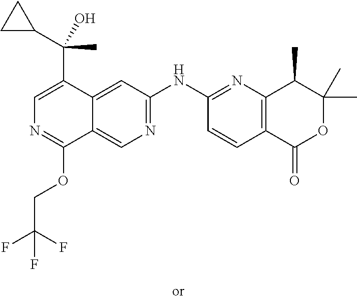 [SMILES: C[C@@H]1c2nc(Nc3cc4c([C@@](C)(O)C5CC5)cnc(OCC(F)(F)F)c4cn3)ccc2C(=O)OC1(C)C] | 517 | ¹H NMR (400 MHz, CD₃OD): δ ppm 9.43 (s, 1 H), 9.31 (s, 1 H), 8.39 (s, 1 H), 8.22 (d, J = 8.8 Hz, 1 H), 7.27 (d, J = 8.8 Hz, 1 H), 5.15-5.09 (m, 2 H), 3.23-3.18 (m, 1 H), 1.72 (s, 3 H), 1.65-1.58 (m, 1 H), 1.51 (s, 3 H), 1.48 (s, 3 H), 1.45 (d, J = 7.2 Hz, 3 H), 0.65-0.51 (m, 4 H). | Scheme 1, Intermediate 3 and Intermediate 49 |
| | 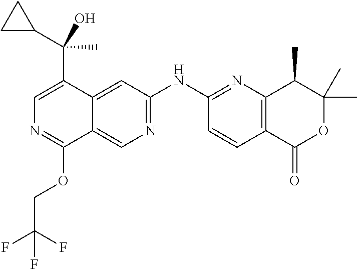 [SMILES: C[C@@H]1c2nc(Nc3cc4c([C@](C)(O)C5CC5)cnc(OCC(F)(F)F)c4cn3)ccc2C(=O)OC1(C)C] | | | |
| 55 | 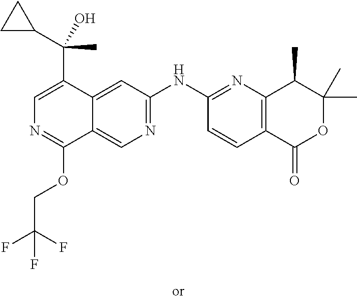 [SMILES: C[C@@H]1c2nc(Nc3cc4c([C@@](C)(O)C5CC5)cnc(OCC(F)(F)F)c4cn3)ccc2C(=O)OC1(C)C] | | ¹H NMR (400 MHz, CD₃OD): δ ppm 9.53-9.49 (m, 1 H), 9.34 (s, 1 H), 8.38-8.33 (m, 1 H), 8.21 (d, J = 8.4 Hz, 1 H), 7.28- 7.24 (m, 1 H), 5.13-5.06 (m, 2 H), 3.18-3.04 (m, 1 H), 1.76 (d, J = 11.2 Hz, 3 H), 1.68- 1.57 (m, 1 H), 1.51 (s, 3 H), 1.46 (d, J = 7.2 Hz, 3 H), 1.42- 1.35 (m, 3 H), 0.70-0.48 (m, 4 H). | Scheme 1, Intermediate 3 and Intermediate 50 |
| | 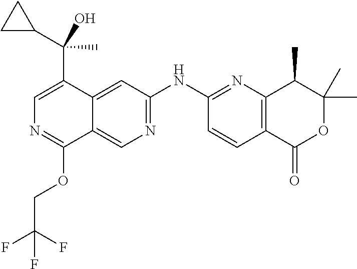 [SMILES: C[C@@H]1c2nc(Nc3cc4c([C@](C)(O)C5CC5)cnc(OCC(F)(F)F)c4cn3)ccc2C(=O)OC1(C)C] | | | |
| 56 | 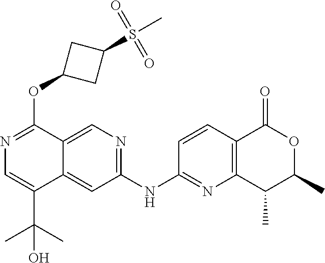 [SMILES: C[C@@H]1OC(=O)c2ccc(Nc3cc4c(C(C)(C)O)cnc(O[C@H]5C[C@@H](S(C)(=O)=O)C5)c4cn3)nc2[C@H]1C] | 527 | ¹H NMR (400 MHz, 6d- DMSO): δ ppm 10.68 (s, 1H), 9.32 (s, 1H), 9.29 (s, 1H), 8.09-8.07 (m, 2H), 7.36 (d, J = 8.8 Hz, 1H), 5.40-5.32 (m, 1H), 5.22 (s, 1H), 4.61-4.54 (m, 1H), 3.88-3.79 (m, 1H), 3.01-2.96 (m, 1H), 2.94 (s, 3H), 2.89-2.82 (m, 2H), 2.07 (s, 1H), 1.69 (s, 6H), 1.45 (d, J = 7.2 Hz, 3H), 1.38 (d, J = 6.8 Hz, 3H). | Scheme 1, Intermediate 1 and Intermediate 51 |
| 57 | 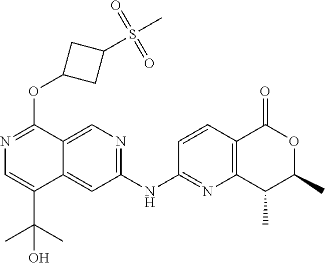 [SMILES: C[C@@H]1OC(=O)c2ccc(Nc3cc4c(C(C)(C)O)cnc(OC5CC(S(C)(=O)=O)C5)c4cn3)nc2[C@H]1C] | 528 | ¹H NMR (400 MHz, CD₃OD) δ ppm 9.40 (s, 1H), 9.27 (s, 1H), 8.15 (s, 1H), 8.13 (s, 1H), 7.32 (d, J = 8.8 Hz, 1H), 5.60 (s, 1H), 4.70-4.56 (m, 1H), 4.44 (t, J = 7.8 Hz, 2H), 4.28-4.11 (m, 2H), 3.33 (s, 6H), 3.05 (s, 4H), 1.82 (s, 6H), 1.57-1.45 (m, 6H). | Scheme 1, Intermediate 1 and Intermediate 54 |
| 58 | 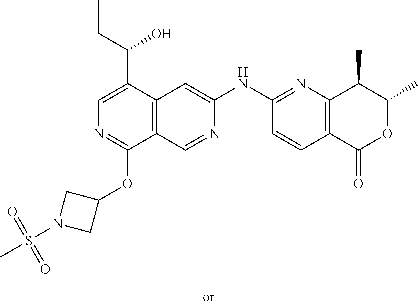 | 528 | ¹H NMR (400 MHz, CD₃OD): δ ppm 10.76 (s, 1H), 9.41 (s, 1H), 8.88 (s, 1H), 8.10 (d, J = 8.8 Hz, 1H), 8.05 (s, 1H), 7.33 (d, J = 8.4 Hz, 1H), 5.62-5.52 (m, 1H), 5.40-5.30 (m, 1H), 5.00-4.90 (m, 1H), 4.60-4.50 (m, 2H), 4.45-4.32 (m, 2H), 4.30-4.20 (m, 2H), 3.15 (s, 3H), 3.15-3.05 (m, 1H), 1.90- 1.80 (m, 9H), 1.49 (d, J = 7.2 Hz, 3H), 1.44 (d, J = 6.4 Hz, 3H), 0.95-0.85 (m, 3H) | Scheme 1, Intermediate 1 and Intermediate 55 |
| | 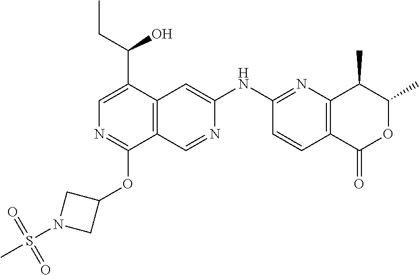 | | | |
| 59 | 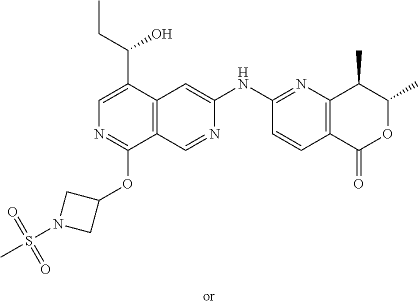 | 528 | ¹H NMR (400 MHz, CD₃OD): δ ppm 10.77 (s, 1H), 9.42 (s, 1H), 8.85 (s, 1H), 8.11 (d, J = 8.8 Hz, 1H), 8.07 (s, 1H), 7.33 (d, J = 8.4 Hz, 1H), 5.62-5.52 (m, 1H), 5.40-5.30 (m, 1H), 5.00-4.90 (m, 1H), 4.60-4.50 (m, 2H), 4.45-4.32 (m, 2H), 4.30-4.20 (m, 2H), 3.15 (s, 3H), 3.15-3.05 (m, 1H), 1.90- 1.80 (m, 9H), 1.49 (d, J = 7.2 Hz, 3H), 1.44 (d, J = 6.4 Hz, 3H), 0.95-0.85 (m, 3H). | Scheme 1, Intermediate 1 and Intermediate 56 |
| | 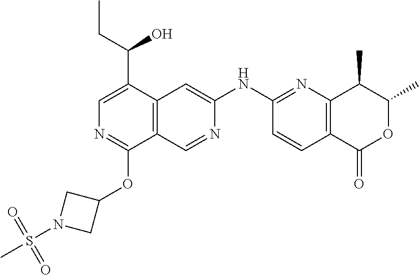 | | | |
| 60 | 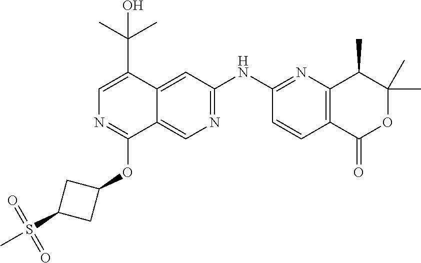 | 541 | ¹H NMR (400 MHz, 6d- DMSO): δ ppm 10.72 (s, 1H), 9.38 (s, 1H), 9.32 (s, 1H), 8.11 (s, 1H), 8.06 (d, J = 8.8 Hz, 1H), 7.31 (d, J = 8.8 Hz, 1H), 5.40-5.33 (m, 1H), 5.21 (s, 1H), 3.86-3.81 (m, 1H), 3.03- 2.98 (m, 1H), 2.94 (s, 3H), 2.89-2.82 (m, 2H), 2.07 (s, 1H), 1.71 (s, 6H), 1.39 (d, J = 13.6 Hz, 6H), 1.33 (d, J = 7.2 Hz, 3H). | Scheme 1, Intermediate 3 and Intermediate 51 |
| 61 | 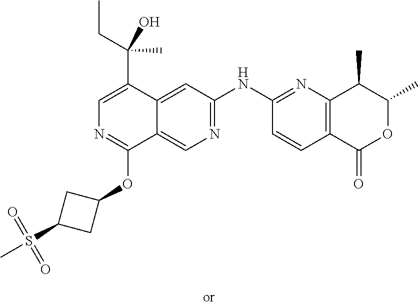 | 541 | ¹H NMR (400 MHz, CD₃OD): δ ppm 9.36 (s, 1H), 9.22 (s, 1H), 8.14 (d, J = 8.4 1H), 8.13 (s, 1H), 7.30 (d, J = 8.4 Hz, 1H), 5.49-5.39 (m, 1H), 4.68- 4.54 (m, 2H), 3.88-3.74 (m, 1H), 3.02-2.96 (m, 2H), 2.93 (s, 3H), 2.75-2.64 (m, 2H), 2.28-2.12 (m, 2H), 1.77 (s, 3H), 1.51 (d, J = 7.2 Hz, 3H), 1.47 (d, J = 6.8 Hz, 3H), 0.80 (t, J = 7.6 Hz, 3H). | Scheme 1, Intermediate 1 and Intermediate 53 |
| | 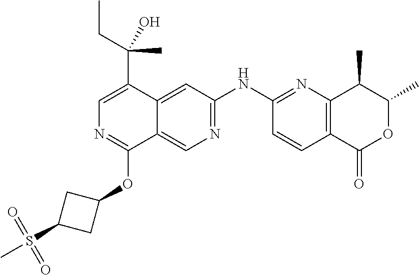 | | | |
| 62 | 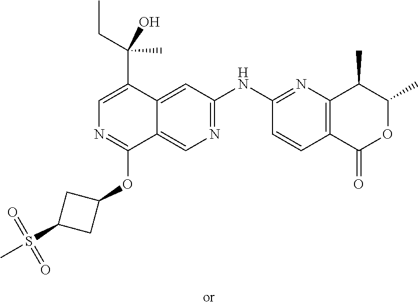 | 541 | ¹H NMR (400 MHz, CD₃OD): δ ppm 9.36 (s, 1H), 9.19 (s, 1H), 8.14 (s, 1H), 8.13 (d, J = 8.8 Hz, 1H), 7.30 (d, J = 8.8 Hz, 1H), 5.48-5.39 (m, 1H), 3.88-3.76 (m, 1H), 3.02- 2.95 (m, 2H), 2.93 (s, 3H), 2.74-2.63 (m, 2H), 2.25- 2.12 (m, 2H), 1.77 (s, 3H), 1.51 (d, J = 7.2 Hz, 3H), 1.47 (d, J = 6.8 Hz, 3H), 0.81 (t, J = 7.2 Hz, 3H). | Scheme 1, Intermediate 1 and Intermediate 52 |
| | 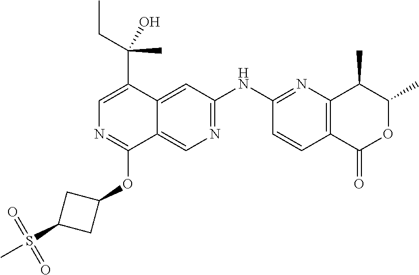 | | | |
| 63 | 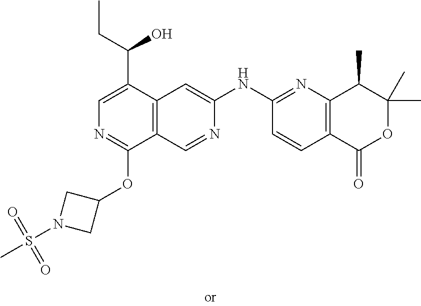 | 542 | ¹H NMR (400 MHz, CD₃OD): δ ppm 10.77 (s, 1H), 9.41 (s, 1H), 8.91 (s, 1H), 8.09 (d, J = 8.8 Hz, 1H), 8.06 (s, 1H), 7.32 (d, J = 8.8 Hz, 1H), 5.62-5.55 (m, 1H), 5.50-5.30 (m, 1H), 5.00-4.90 (m, 1H), 4.50-4.40 (m, 2H), 4.20-4.10 (m, 2H), 3.20-3.05 (m, 4H), 1.90-1.80 (m, 2H), 1.60-1.30 (m, 9H), 1.00-0.85 (m, 3H) | Scheme 1, Intermediate 3 and Intermediate 56 |
| | 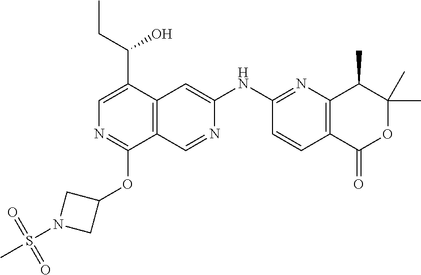 | | | |
| 64 | 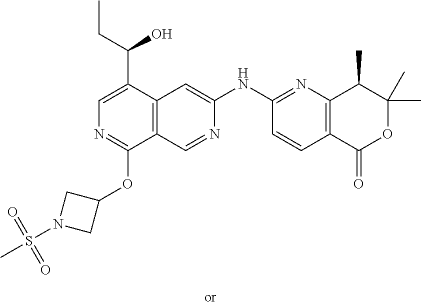 | 542 | ¹H NMR (400 MHz, CD₃OD): δ ppm 10.78 (s, 1H), 9.42 (s, 1H), 8.96 (s, 1H), 8.09 (d, J = 8.8 Hz, 1H), 8.04 (s, 1H), 7.33 (d, J = 8.8 Hz, 1H), 5.62-5.55 (m, 1H), 5.50-5.30 (m, 1H), 5.00-4.90 (m, 1H), 4.50-4.40 (m, 2H), 4.20-4.10 (m, 2H), 3.20-3.05 (m, 4H), 1.90-1.80 (m, 2H), 1.60-1.30 (m, 9H), 1.00-0.85 (m, 3H) | Scheme 1, Intermediate 3 and Intermediate 55 |
| | 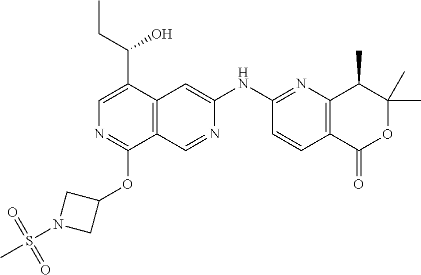 | | | |
| 65 | 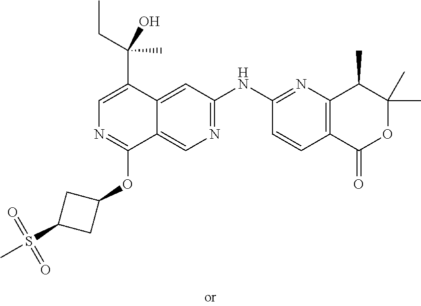 | 555 | ¹H NMR (400 MHz, CD₃OD): δ ppm 9.39 (s, 1H), 9.34 (s, 1H), 8.13 (s, 1H), 8.11 (d, J = 8.4 Hz, 1H), 7.21 (d, J = 8.8 Hz, 1H), 5.48-5.38 (m, 1H), 3.88-3.77 (m, 1H), 3.12- 3.04 (m, 1H), 3.02-2.95 (m, 2H), 2.93 (s, 3H), 2.74-2.64 (m, 2H), 2.32-2.12 (m, 2H), 1.77 (s, 3H), 1.52 (s, 3H), 1.45 (s, 3H), 1.38 (d, J = 7.2 Hz, 3H), 0.81 (t, J = 7.6 Hz, 3H). | Scheme 1, Intermediate 1 and Intermediate 53 |
| | 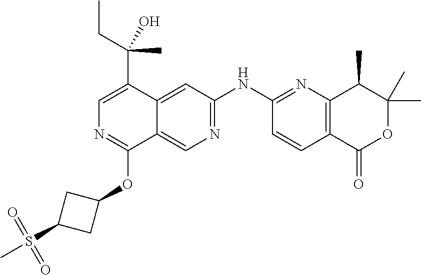 | | | |
| 66 | 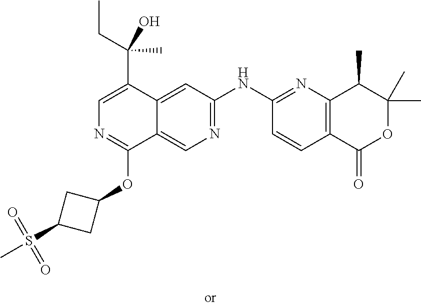 | 555 | ¹H NMR (400 MHz, CD₃OD): δ ppm 9.35 (s, 1H), 9.33 (s, 1H), 8.15 (s, 1H), 8.11 (d, J = 8.8 Hz, 1H), 7.22 (d, J = 8.8 Hz, 1H), 5.49-5.38 (m, 1H), 3.88-3.77 (m, 1H), 3.08- 3.04 (m, 1H), 3.01-2.95 (m, 2H), 2.93 (s, 3H), 2.74-2.62 (m, 2H), 2.20 (m, 2H), 1.78 (s, 3H), 1.52 (s, 3H), 1.45 (s, 3H), 1.40 (d, J = 7.2 Hz, 3H), 0.82 (t, J = 7.6 Hz, 3H). | Scheme 1, Intermediate 1 and Intermediate 52 |
| | 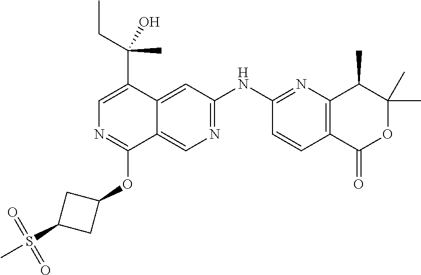 | | | |
| 67 | 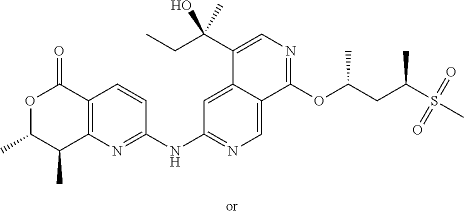 | 557 | ¹H NMR (400 MHz, CD3OD): δ ppm 9.35 (s, 1 H), 9.17 (s, 1 H), 8.24-8.05 (m, 2 H), 7.30 (d, J = 8.4 Hz, 1 H), 5.76-5.60 (m, 1 H), 4.72-4.49 (m, 1 H), 3.42-3.34 (m, 1 H), 3.07-2.98 (m, 1 H), 2.94 (s, 3 H), 2.48- 2.36 (m, 1 H), 2.26-2.07 (m, 3 H), 1.77 (s, 3 H), 1.53-1.45 (m, 12 H), 0.87-0.75 (m, 3 H). | Scheme 1, Intermediate 1 and Intermediate 57 |
| | 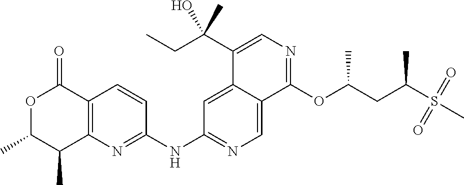 | | | |
| 68 | 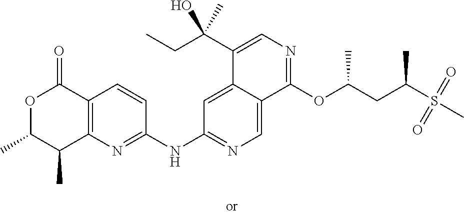 | 557 | ¹H NMR (400 MHz, CD3OD): δ ppm 9.35 (s, 1 H), 9.21 (s, 1 H), 8.25-8.05 (m, 2 H), 7.29 (d, J = 8.8 Hz, 1 H), 5.80-5.59 (m, 1 H), 4.66-4.58 (m, 1 H), 3.43-3.34 (m, 1 H), 3.10-3.00 (m, 1 H), 2.94 (s, 3 H), 2.46- 2.37 (m, 1 H), 2.29-2.07 (m, 3 H), 1.76 (s, 3 H), 1.52-1.45 (m, 12 H), 0.85-0.74 (m, 3 H). | Scheme 1, Intermediate 1 and Intermediate 58 |
| | 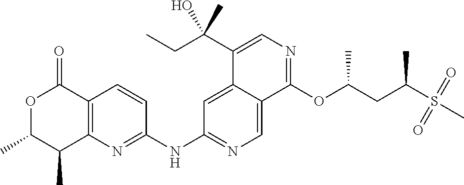 | | | |
| 69 | 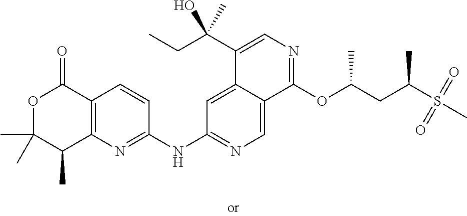 | 571 | ¹H NMR (400 MHz, CD3OD): δ ppm 9.34 (d, J = 6.4 Hz, 2 H), 8.17 (s, 1 H), 8.11 (d, J = 8.8 Hz, 1 H), 7.22 (d, J = 8.8 Hz, 1 H), 5.77-5.54 (m, 1 H), 3.42-3.34 (m, 1 H), 3.10-3.02 (m, 1 H), 2.93 (s, 3 H), 2.47- 2.37 (m, 1 H), 2.29-2.08 (m, 3 H), 1.78 (s, 3 H), 1.53-1.48 (m, 9 H), 1.45 (s, 3 H), 1.40 (d, J = 7.2 Hz, 3 H), 0.87-0.78 (m, 3 H). | Scheme 1, Intermediate 3 and Intermediate 57 |
| | 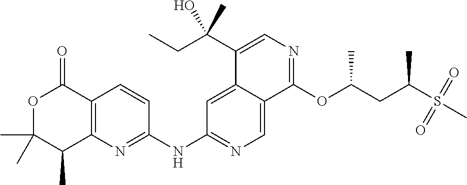 | | | |
| 70 | 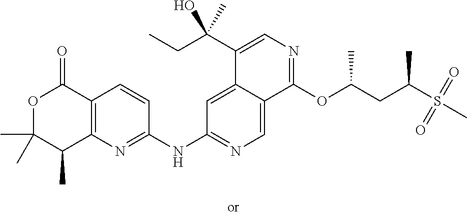 | 571 | ¹H NMR (400 MHz, CD3OD): δ ppm 9.41 (s, 1 H), 9.34 (s, 1 H), 8.15 (s, 1 H), 8.11 (d, J = 8.8 Hz, 1 H), 7.21 (d, J = 8.8 Hz, 1 H), 5.84-5.59 (m, 1 H), 3.44-3.34 (m, 1 H), 3.13-3.04 (m, 1 H), 2.94 (s, 3 H), 2.46- 2.38 (m, 1 H), 2.34-2.03 (m, 3 H), 1.78 (s, 3 H), 1.53-1.48 (m, 9 H), 1.46 (s, 3 H), 1.38 (d, J = 7.2 Hz, 3 H), 0.85-0.77 (m, 3 H). | Scheme 1, Intermediate 3 and Intermediate 58 |
| | 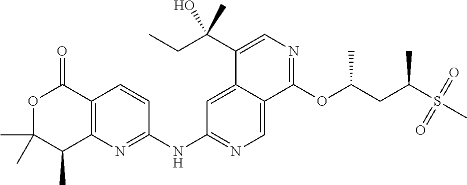 | | | |

Included in the present teachings are pharmaceutically acceptable salts of the compounds disclosed herein (including compounds 1-70 disclosed in Table 1 and Exemplification) as well as the corresponding charge neutral form e.g., free base.

Another embodiment of the disclosure is a compound disclosed herein, including a compound of Formulae I, II, III, IV(A), IV(B), V, VI or VII or a compound in Table 1 or in the exemplification or a pharmaceutically acceptable salt of any of the foregoing, in which one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of compounds with deuterium enrichment at the site of enrichment with the number of compounds having hydrogen or deuterium at the site of enrichment.

### Definitions

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge et al. describes pharmacologically acceptable salts in J. Pharm. Sci. (1977) 66:1-19. Compounds of the present teachings with basic groups can form pharmaceutically acceptable salts with pharmaceutically acceptable acid(s). Suitable pharmaceutically acceptable acid addition salts of the compounds described herein include salts of inorganic acids (such as hydrochloric acid, hydrobromic, phosphoric, nitric, and sulfuric acids) and of organic acids (such as acetic acid, benzenesulfonic, benzoic, methanesulfonic, and p-toluenesulfonic acids). Compounds of the present teachings with acidic groups can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

The term “alkyl” used alone or as part of a larger moiety, such as “alkoxy”, “hydroxyalkyl” and the like, means a saturated aliphatic straight-chain or branched monovalent hydrocarbon radical. Unless otherwise specified, an alkyl group typically has 1 to 6 carbon atoms (C₁₋₆ alkyl), (i.e., 1, 2, 3, 4, 5 or 6) alternatively, 1 to 3 carbon atoms (C₁₋₃ alkyl) (i.e., 1, 2 or 3). “C₁₋₆ alkyl” is means a radical having 1 to 6 carbon atoms in a linear or branched arrangement, such as methyl, ethyl, propyl, isopropyl, butyl, isobutyl, tert-butyl, and the like.

The term “alkylene” means a bivalent alkyl group, for example C₁-C₆ alkyl is a group —(CH₂)n- where n is 1 to 6, C₁-C₃ alkyl is a group —(CH₂)n- where n is 1 to 3, unless otherwise specified.

“Cycloalkyl” means a saturated aliphatic cyclic hydrocarbon ring radical. Unless otherwise specified, a cycloalkyl has 3 to 8 ring carbon atoms (C₃₋₈ cycloalkyl) (i.e., 3, 4, 5, 6, 7, or 8), alternatively, 3 to 6 ring carbon atoms (C₃₋₆ cycloalkyl) (i.e., 3, 4, 5, or 6), alternatively, 3 to 5 carbon atoms (C₃₋₅ cycloalkyl) (i.e., 3, 4, or 5). “C₃₋₆ Cycloalkyl” means a radical having from 3 to 6 carbon atoms arranged in a monocyclic ring. A C₃₋₆ cycloalkyl includes cyclopropyl, cyclobutyl, cyclopentyl and cyclohexyl. A C₃₋₅ cycloalkyl includes cyclopropyl, cyclobutyl, and cyclopentyl. The term “cycloalkyl” also means to include a saturated aliphatic cyclic hydrocarbon fused to an aromatic group such as in Formula (I) wherein “Y and B taken together form a C₅₋₆cycloalkyl.”

The term “halogen” or “halo” means fluorine or fluoro (F), chlorine or chloro (Cl), bromine or bromo (Br), or iodine or iodo (I).

The term “heterocycle” refers to a monocyclic non-aromatic ring radical containing unless otherwise specified, 3 to 8 ring atoms (i.e., “3, 4, 5, 6, 7, or 8 membered”) selected from carbon atom and 1 or 2 heteroatoms. Each heteroatom is independently selected from nitrogen, quaternary nitrogen, oxidized nitrogen (e.g., NO); oxygen; and sulfur, including sulfoxide and sulfone. For example, 4-6 membered heterocycle containing nitrogen refers to a monocyclic non-aromatic ring radical containing 2-5 carbon atoms and 1 or 2 nitrogen atoms; 4-6 membered heterocycle containing oxygen atoms refers to a monocyclic non-aromatic ring radical containing 2-5 carbon atoms and 1 or 2 oxygen. Representative heterocycles include azetidinyl, morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like.

The term “hydroxyl” or “hydroxy” refers to the group OH.

The term “substituted”, whether preceded by the term “optionally” or not, refers to the replacement of a hydrogen substituent in a given structure with a non-hydrogen substituent. Thus, for example, a substituted alkyl is an alkyl wherein at least one non-hydrogen substituent is in the place of a hydrogen substituent on the alkyl group. To illustrate, monofluoroalkyl is an alkyl substituted with a fluoro substituent, and difluoroalkyl is an alkyl substituted with two fluoro substituents. It should be recognized that if there is more than one substitution on a substituent, each non-hydrogen substituent can be identical or different (unless otherwise stated).

If a group is described as “optionally substituted”, the group can be either (1) not substituted or (2) substituted.

If a group is described as optionally substituted with up to a particular number of non-hydrogen substituents, that group can be either (1) not substituted; or (2) substituted by up to that particular number of non-hydrogen substituents or by up to the maximum number of substitutable positions on the substituent, whichever is less. Thus, for example, if a group is described as a cycloalkyl optionally substituted with up to 3 non-hydrogen substituents, then any cycloalkyl with less than 3 substitutable positions would be optionally substituted by up to only as many non-hydrogen substituents as the cycloalkyl has substitutable positions.

The term “sulfone” refers to the group —S(O)₂—.

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are non-superimposable mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs).

“Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two or more stereoisomers are depicted by their chemical names or structures, and the names or structures are connected by an “or”, one or the other of the two or more stereoisomers is intended, but not both. The enrichment of one stereoisomer relative to the other is as indicated above.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds described herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” or “first eluting isomer” in the Experimental section refers to an intended reaction product compound obtained from a chromatography separation/purification that elutes earlier than a second intended reaction product compound from the same preceding reaction. The second intended product compound is referred to as “peak 2” or “second eluting isomer”.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

### Use Embodiments

Compounds of the disclosure are MAP4K1 inhibitors. The use of the word “inhibitor” means that a compound or a pharmaceutically acceptable salt thereof inhibits activity of MAP4K1. By “inhibit” herein is meant to decrease the activity of the target enzyme as compared to the activity of that enzyme in the absence of the inhibitor. In some alternatives, the term “inhibit” means a decrease in MAP4K1 activity of at least 5%, at least 10%, at least 20%, at least 50%, at least 60%, at least 79%, at least 80%, at least 90% or at least 95%. In other alternatives, inhibit means a decrease in MAP4K1 activity of 5% to 25%, 25% to 50%, 50 to 70%, 75 to 100%. In some embodiments, inhibit means a decrease in MAP4K1 activity about 95% to 100%, e.g., a decrease in activity of 95%, 96%, 97%, 98%, 99%, or 100%. Such decreases can be measured using a variety of techniques that would be recognizable by one of skill in the art, including in vitro kinase assays.

Compounds of the disclosure are selective MAP4K1 inhibitors. As used herein, a “selective MAP4K1 inhibitor” refers to a compound or a pharmaceutically acceptable salt thereof, which has the ability to selectively inhibit MAP4K1 kinase over other targets. More specifically, a selective MAP4K1 inhibitor has the ability to selectively inhibit MAP4K1 over another kinase. A selective MAP4K1 inhibitor has the ability to selectively reduce target signaling activity relative to off-target signaling activity, via direct or indirect interaction with the target. The ability to selectively target MAP4K1 with a compound or pharmaceutically acceptable salt thereof provides advantages in terms of improved potency, less off-target activity and an increased probability of clinical success in comparison with a non-selective compound or salt.

A MAP4K1 inhibitor that selectively inhibits MAP4K1 may have an activity that is at least 2-fold relative to another kinase (e.g., at least 10-fold; at least 15-fold; at least 20-fold; at least 30-fold; at least 40-fold selectivity; at least 50-fold; at least 60-fold; at least 70-fold; at least 80-fold; at least 90-fold; at least 100-fold; at least 125-fold; at least 150-fold; at least 175-fold; or at least 200-fold. In some alternatives, a selective MAP4K1 inhibitor exhibits at least 15-fold selectivity over another kinase, e.g., LCK and MAP4K family members (MAP4K4 (HGK) and MAP4K3 (GLK)). In some alternatives, the selective MAP4K1 inhibitors are selective over EGFR and L858R/T790M EGFR. In some alternatives, the selective MAP4K1 inhibitors of the disclosure are selective over BTK. In some alternatives, the selective MAP4K1 inhibitors of the disclosure are selective over JNK.

The disclosure provides methods of modulating (e.g., inhibiting) MAP4K1 activity in a subject in need thereof, said method comprising administering to the subject a compound provided herein, or a pharmaceutically acceptable salt thereof. In certain embodiments, the compounds of the disclosure, or pharmaceutically acceptable salts thereof, are useful for therapeutic administration to enhance, stimulate and/or increase immunity in subjects in need thereof, e.g., in cancer patients or patients with viral infection. In some instances, the compounds of the disclosure, or pharmaceutically acceptable salts thereof reduce, inhibit, or otherwise diminish pSLP76.

In some instances, the compounds of the disclosure, or pharmaceutically acceptable salts thereof, are useful for therapeutic administration to enhancing at least one of activation, priming, migration, proliferation, survival and cytolytic activity of T cells relative to prior to administration. In certain aspects, T cell activation is characterized by enhanced levels of IL-2, IFN-gamma, or granzyme B production by T cells relative to prior to administration of the compound or pharmaceutically acceptable salt thereof. In some instances, the compounds of the disclosure, or pharmaceutically acceptable salts thereof, are useful for therapeutic administration to induce a change in cell cycle or cell viability.

In some instances, the compounds of the disclosure, or pharmaceutically acceptable salts thereof, are useful for improving function of T effector cells. In some instances, the compounds of the disclosure, or pharmaceutically acceptable salts thereof, are useful for inhibiting the suppressive effects of T regulatory cells or improving the T cell response to immune suppressive factors including adenosine and PGE2. In some instances, the compounds of the disclosure, or pharmaceutically acceptable salts thereof, are useful for increasing the frequency of CD8+ tumor infiltrating lymphocytes (TILS). In some instances, the compounds of the disclosure, or pharmaceutically acceptable salts thereof, are useful for enhancing CD3+/Treg ratios.

In some instances, the compounds of the disclosure, or pharmaceutically acceptable salts thereof, are useful for enhancing cytokines. In some instances, the compounds of the disclosure, or pharmaceutically acceptable salts thereof, are useful for enhancing cytokines with no impact on IL-6. In some instances, the compounds of the disclosure, or pharmaceutically acceptable salts thereof, indirectly inhibit the growth of cancer cells. In some instances, the compounds of the disclosure, or pharmaceutically acceptable salts thereof, are useful for priming of the immune response (i.e., vaccines) to tumors or viruses for booting or generating anti-viral/anti-tumor immunity. In one instance, the compounds of the disclosure, or pharmaceutically acceptable salts thereof, are used for enhancing or boosting response to a vaccine (such as a cancer vaccine or a personalized cancer vaccine (PCV)) or a CAR-T cell therapy.

Methods of treating a MAP4K1-dependent disease or disorder can include administering to a subject in need thereof a therapeutically effective amount of a compound provided herein, or a pharmaceutically acceptable salt thereof. For example, the MAP4K1-dependent disease or disorder is a cancer. The term “cancer” encompasses all forms of cancer including, but not limited to, all forms of carcinoma, melanomas, blastomas, sarcomas, lymphomas, leukemias. In some embodiments, cancer includes metastatic forms. Additionally, the disclosure includes refractory or recurrent malignancies whose growth may be inhibited using the compounds of the disclosure. For the uses described herein, any of the compounds of the disclosure, or pharmaceutically acceptable salts thereof, may be used alone or in combination with other therapeutic agents.

In some embodiments, the treatment results in a sustained response in the subject after cessation of the treatment. “Sustained response” refers to the sustained effect on reducing tumor growth after cessation of a treatment. For example, the tumor size may remain the same or smaller as compared to the size at the beginning of the administration phase. In some embodiments, the sustained response has a duration at least the same as the treatment duration, at least 1.5×, 2.0×, 2.5×, or 3.0× length of the treatment duration. The treatment methods disclosed herein may result in a partial or complete response.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started. As used herein, “overall response rate” (ORR) refers to the sum of complete response (CR) rate and partial response (PR) rate.

The treatment methods disclosed herein can lead to an increase in progression free survival and overall survival of the subject administered the selective MAP4K1 inhibitor. As used herein, “progression free survival” (PFS) refers to the length of time during and after treatment during which the disease being treated (e.g., cancer) does not get worse. Progression-free survival may include the amount of time subjects have experienced a complete response or a partial response, as well as the amount of time subjects have experienced stable disease.

As used herein, “overall survival” (OS) refers to the percentage of subjects in a group who are likely to be alive after a particular duration of time.

In some embodiments, cancers treatable with compounds of the disclosure or pharmaceutically acceptable salt thereof, include colon cancer, pancreatic cancer, breast cancer, prostate cancer, lung cancer, ovarian cancer, cervical cancer, renal cancer, bladder cancer, stomach cancer, liver cancer, gastric cancer, cancer of the head and neck, lymphoma, leukemia, urothelial carcinoma, merkel cell carcinoma, gastroesophageal junction carcinoma, esophageal squamous cell carcinoma, skin squamous cell carcinoma and melanoma.

In some embodiments, cancers treatable with compounds of the disclosure or pharmaceutically acceptable salts thereof include colon cancer, pancreatic cancer, breast cancer, prostate cancer, lung cancer, ovarian cancer, cervical cancer, renal cancer, bladder cancer, stomach cancer, liver cancer, cancer of the head and neck, lymphoma, leukemia, and melanoma.

In some embodiments, cancers that are treatable using the compounds of the disclosure or pharmaceutically acceptable salts thereof include, but are not limited to, solid tumors, including prostate cancer, colon cancer, esophageal cancer, endometrial cancer, ovarian cancer, uterine cancer, renal cancer, hepatic cancer, pancreatic cancer, gastric cancer, breast cancer, lung cancer, head and neck cancer, thyroid cancer, brain cancer, and bladder cancer and hematological cancer, including lymphoma, leukemia (chronic and acute forms) such as ALL, AML, CLL, CML, DLBCL, mantle cell lymphoma, Non-Hodgkin's lymphoma (NHL), including relapsed or refractory NHL and recurrent follicular, Hodgkin's lymphoma and multiple myeloma, and myeloproliferative diseases.

In some embodiments, diseases and indications that are treatable using the compounds of the disclosure or pharmaceutically acceptable salts thereof include, but are not limited to hematological cancer, sarcomas, respiratory cancer, gastrointestinal cancer, genitourinary tract cancer, liver cancer, bone cancer, nervous system cancer, gynecological cancer, and skin cancer.

Exemplary hematological cancer includes, for example, lymphomas and leukemias such as ALL, AML, acute promyelocyte leukemia (APL), CLL, CML, DLBCL, mantle cell lymphoma, Non-Hodgkin lymphoma (NHL), including Primary mediastinal B-cell lymphoma (PMBCL), relapsed or refractory NHL, recurrent follicular, and primary CNS lymphoma, Hodgkin's lymphoma, myeloproliferative diseases, including, primary myelofibrosis (PMF), polycythemia vera (PV), essential thrombocytosis (ET), myelodysplasia syndrome (MDS), T-cell acute lymphoblastic lymphoma (T-ALL), multiple myeloma, cutaneous T-cell lymphoma, Waldenstrom's Macroglubulinemia, hairy cell lymphoma, chronic myelogenic lymphoma, and Burkitt's lymphoma.

Exemplary sarcoma includes, for example, chondrosarcoma, Ewing's sarcoma, Kaposi's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, sarcoma of the soft tissue, and teratoma.

Exemplary respiratory tract cancer includes, for example, lung cancer such as non-small cell lung cancer (NSCLC), small cell lung cancer, epidermoid cancer, bronchogenic carcinoma, including squamous cell, undifferentiated small cell, undifferentiated large cell, adenocarcinoma, alveolar (bronchiolar) carcinoma, bronchial adenoma, chondromatous hamartoma, mesothelioma, and pleuropulmonary blastoma.

Exemplary gastrointestinal cancer includes, for example, cancers of the esophagus, including squamous cell carcinoma, adenocarcinoma, leiomyosarcoma, and lymphoma; stomach, including carcinoma, lymphoma, and leiomyosarcoma; pancreas, including ductal adenocarcinoma, insulinoma, glucagonoma, gastrinoma, carcinoid tumors, and vipoma; small instestine, including adenocarcinoma, lymphoma, carcinoid tumors, Kaposi's sarcoma, leiomyoma, hemangioma, lipoma, neurofibroma, and fibroma; large intestine, including adenocarcinoma, tubular adenoma, villous adenoma, hamartoma, and leiomyoma; colon; and gall bladder, including adenocarcinoma; and intestinal type and diffuse type gastric adenocarcinoma, rectum carcinoma, familiar adenomatous polyposis carcinoma and hereditary non-polyposis colorectal cancer.

Exemplary genitourinary tract cancer includes, for example, cancers of the kidney, including adenocarcinoma, Wilm's tumor [nephroblastoma], renal cell carcinoma, urothelial carcinoma, juxtaglomerular cell tumor (reninoma), angiomyolipoma, renal oncocytoma, Bellinio duct carcinoma, clear-cell sarcoma of the kidney, and mesoblastic nephroma; adrenal gland; renal pelvis; bladder, including transitional cell carcinoma, squamous cell carcinoma, adenocarcinoma, sarcoma, and small cell carcinoma; urethra, including squamous cell carcinoma, transitional cell carcinoma, and adenocarcinoma; prostate, including adenocarcinoma, sarcoma, and carcinoma; testis, including seminoma, teratoma, embryonal carcinoma, teratocarcinoma, choriocarcinoma, sarcoma, interstitial cell carcinoma, fibroma, fibroadenoma, adenomatoid tumors, and lipoma; penis; and pancreas.

Exemplary liver cancer includes, for example, hepatoma, including hepatocellular carcinoma, cholangiocarcinoma, hepatoblastoma, angiosarcoma, hepatocellular adenoma, biliary tract cancer, and hemangioma.

Exemplary bone cancer includes, for example, osteogenic sarcoma, fibrosarcoma, malignant fibrous histiocytoma, chondrosarcoma, Ewing's sarcoma, malignant lymphoma, including reticulum cell sarcoma, multiple myeloma, malignant giant cell tumor chordoma, osteochronfroma, including osteocartilaginous exostoses, benign chondroma, chondroblastoma, chondromyxofibroma, osteoid osteoma, and giant cell tumors

Exemplary nervous system cancer includes, for example, cancer of the skull, including osteoma, hemangioma, granuloma, xanthoma, and osteitis deformans; meninges including, meningioma, meningiosarcoma, and gliomatosis; brain, including astrocytoma, meduoblastoma, glioma, ependymoma, germinoma (pinealoma), neuroectodermal tumor, glioblastoma, glioblastoma multiform, oligodendroglioma, schwannoma, retinoblastoma, congenital tumors, brain stem and hypopthamic glioma; and spinal cord, including neurofibroma, meningioma, glioma, and sarcoma; as well as neuroblastoma and Lhermitte-Duclos disease.

Exemplary gynecological cancer includes, for example, cancer of the uterus, including endometrial carcinoma; cervix, including cervical carcinoma, pre-tumor cervical dysplasia, squamouse cell carcinoma, adenocarcinoma, adenosquamous carcinoma, small cell carcinoma, neuroendocrine tumor, glassy cell carcinoma and villoglandular adenocarcinoma; ovaries, including ovarian carcinoma (serous cystadenocarcinoma, mucinous cystadenocarcinoma, unclassified carcinoma, endometroid tumor, high-grade serous carcinoma), granulosa-thecal cell tumors, Sertoli-Leydig cell tumors, dysgerminoma, malignant teratoma, and arrhenoblastoma; vulva (squamous cell carcinoma, intraepithelial carcinoma, adenocarcinoma, fibrosarcoma, and melanoma; vagina, including clear cell carcinoma, squamous cell carcinoma, and botryoid sarcoma (embryonal rhabdomyosarcoma); labia; and fallopian tubes.

Exemplary skin cancer includes, for example, melanoma, sebaceous gland carcinoma, basal cell carcinoma, squamous cell carcinoma, Kaposi's sarcoma, Merkel cell skin cancer, moles dysplastic nevi, lipoma, angioma, dermatofibroma, and keloids.

Examples of breast cancer include, for example, ER+/HER2− breast cancer, triple-negative breast cancer (TNBC), invasive ductal carcinoma, invasive lobular carcinoma, ductal carcinoma in situ, and lobular carcinoma in situ.

Exemplary head and neck cancer includes, for example, glioblastoma, melanoma, rhabdosarcoma, lymphosarcoma, osteosarcoma, squamous cell carcinomas, adenocarcinomas, oral cancer, throat cancer, including oropharyngeal cancer, laryngeal cancer, nasopharyngeal cancer, nasal and paranasal cancer, salivary gland cancer, mouth cancer, eye cancer, acoustic neuroma, pituitary adenoma, hypopharngx, and thyroid (medullary and papillary) and parathyroid cancer.

Other cancers include, for example, sweat gland cancer, spinal axis tumor, chest cancer, sickle cell anemia, and environmentally induced cancers including those induced by asbestos.

In some embodiments, compounds of the disclosure or pharmaceutically acceptable salts thereof are for the treatment of advanced melanoma, advanced NSCLC, or advanced head and neck squamous cell cancer, including where the subject was refractory to, or had a partial response to, immune checkpoint inhibitor therapy.

In some instances, the MAP4K1-dependent disease or disorder is a viral infection, such as infection caused by hepatitis B virus (HBV), hepatitis C virus (HCV), human papilloma virus (HPV), cytomegalovirus (CMV), herpes simplex virus (HSV), Epstein-Barr virus (EBV), varicella zoster virus, coxsackie virus, and human immunodeficiency virus (HIV).

### Combination Therapies

Compounds of the disclosure or pharmaceutically acceptable salts thereof can be administered as the sole pharmaceutical agent or in combination with one or more other anti-cancer agents for the treatment of cancel, where the combination causes no unacceptable adverse effects. In some embodiments, the other anti-cancer agents are immune-oncology agent, anticancer agents that are enzyme/protein/receptor inhibitors, radiation or chemotherapy.

Compounds of the disclosure or pharmaceutically acceptable salts thereof can be co-formulated with an immuno-oncology agent. Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human. In another aspect, the antibody is a bispecific antibody.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators, in some instances immune checkpoint inhibitors).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTfiR, LIGHT, DcR3, HVEM, VEGI/TL1A, TRAMP/DR3, EDAR, EDA1, XEDAR, EDA2, TNFR1, Lymphotoxin α/TNPβ, TNFR2, TNF a, LT R, Lymphotoxin a 1β2, FAS, FASL, RELT, DR6, TROY, NGFR.

In one aspect, T cell responses can be stimulated by a combination of a compound of the disclosure and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In one aspect, compounds of the disclosure or pharmaceutically acceptable salts thereof can be administered in combination with at least one other immune checkpoint inhibitor. In other aspects, compounds of the disclosure or pharmaceutically acceptable salts thereof can be administered for the treatment of immune checkpoint inhibitor-resistant NSCLC, including where the subject is refractory to, or had a partial response to, platinum and/or paclitaxel and/or docetaxel therapy. Optionally, compounds of the disclosure or pharmaceutically acceptable salts thereof can be administered in combination with at least one other anti-cancer agent, such as paclitaxel, docetaxel or platinum anticancer therapy. Compounds of the disclosure or pharmaceutically acceptable salts thereof can be administered for the first line treatment of NSCLC expressing high PD-L1 (≥50% Tumor Proportion Score (TPS), wild-type EGFR, or wild-type ALK).

Other agents that can be combined with compounds of the disclosure for the treatment of cancer include antagonists of inhibitory receptors on NK cells or agonists of activating receptors on NK cells. For example, compounds of the disclosure can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 or FPA-008.

In another aspect, compounds of the disclosure or pharmaceutically acceptable salts thereof can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In some embodiments, the immuno-oncology agent is a CTLA-4 antagonist, such as an antagonistic CTLA-4 antibody. Suitable CTLA-4 antibodies include, for example, YERVOY (ipilimumab) or tremelimumab. In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgGl, called AMP-224

In another aspect, the immuno-oncology agent is a PD-L1 antagonist, such as an antagonistic PD-L1 antibody. Suitable PD-L1 antibodies include, for example, TECENTRIQ (atezolizumab) (RG7446; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WOl 1/028683).

In another aspect, the immuno-oncology agent is an IDO antagonist. Suitable IDO antagonists include, for example, INCB-024360 (WO2006/122150, WO07/75598, WO08/36653, WO08/36642), indoximod, or NLG-919 (WO09/73620, WO09/1156652, WOl1/56652, WO12/142237).

In another aspect, the immuno-oncology agent is an OX40 agonist, such as an agonistic OX40 antibody. Suitable OX40 antibodies include, for example, MEDI-6383 or MEDI-6469. In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WOl 1/109400).

The compounds of the disclosure or pharmaceutically acceptable salts thereof can be used in combination with anticancer agents that are enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the disclosure or pharmaceutically acceptable salts thereof can be used in combination with one or more other enzyme/protein/receptor inhibitors for the treatment of cancer. For example, the compounds of the disclosure can be combined with one or more inhibitors of the following kinases for the treatment of cancer: Akt1, Akt2, Akt3, TGF-βPv, PKA, PKG, PKC, CaM-kinase, phosphorylase kinase, MEKK, ERK, MAPK, mTOR, EGFR, HER2, HER3, HER4, INS-R, IGF-1R, IR-R, PDGFotR, PDGFpR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK, and B-Raf.

In some embodiments, the compounds of the disclosure or pharmaceutically acceptable salts thereof can be combined with one or more of the following inhibitors for the treatment of cancer. Non-limiting examples of inhibitors that can be combined with the compounds of the disclosure or pharmaceutically acceptable salts thereof for treatment of cancers include an FGFR inhibitor (FGFR1, FGFR2, FGFR3 or FGFR4, e.g., fisogatinib, AZD4547, BAY 1187982, ARQ087, BGJ398, BIBF1120, TKI258, lucitanib, dovitinib, TAS-120, J J-42756493, Debiol347, INCB54828, INCB62079, and INCB63904), a JAK inhibitor (JAK1 and/or JAK2, e.g., ruxolitinib, baricitinib, or itacitinib (INCB39110)), an IDO inhibitor (e.g., epacadostat and NLG919), an LSD1 inhibitor (e.g., GSK2979552, INCB59872 and INCB60003), a TDO inhibitor, a PI3K-delta inhibitor (e.g., INCB50797 and INCB50465), a PI3K-gamma inhibitor such as a PI3K-gamma selective inhibitor (eganelisib) or a dual PI3K-delta/gamma selective inhibitor (duvelisib), a CSF1R inhibitor (e.g., PLX3397 and LY3022855), a TAM receptor tyrosine kinases (Tyro-3, Ax1, and Mer), an angiogenesis inhibitor (Such as Avastin (bevacizumab)), an interleukin receptor inhibitor, bromo and extra terminal family members inhibitors (for example, bromodomain inhibitors or BET inhibitors such as OTX015, CPI-0610, INCB54329, and INCB57643), and an adenosine receptor antagonist or combinations thereof. Inhibitors of HDAC such as panobinostat and vorinostat can be combined with the compounds of the disclosure.

Inhibitors of c-Met such as onartumzumab, tivantnib, and capmatinib (INC-280) be combined with the compounds of the disclosure. Inhibitors of BTK such as ibrutinib can be combined with the compounds of the present disclosure. Inhibitors of mTOR such as rapamycin, sirolimus, temsirolimus, and everolimus can be combined with the compounds of the present disclosure. Inhibitors of Raf, such as vemurafenib and dabrafenib can be combined with the compounds of the present disclosure. Inhibitors of MEK such as trametinib, selumetinib and GDC-0973 can be combined with the compounds of the present disclosure. Inhibitors of KIT, including avapritinib, BLU-263, imatinib, sunitinib, regorafenib, ripritinib (DCC2618), PLX9486, PLX3397, crenolanib, CDX-0158, CDX-0159. Inhibitors of RET including pralsetinib, selperctinib, alectinib, levatinib, cabozantinib, BOS172738 (DS-5010), SL-1001, TPX-0046, sitravatinib (MGCD516), and RXDX-105. Inhibitors of Hsp90 (e.g., tanespimycin), cyclin dependent kinases (e.g., palbociclib), PARP (e.g., olaparib) and Pim kinases (LGH447, INCB053914, and SGI-1776) can also be combined with compounds of the disclosure.

Compounds of the disclosure or pharmaceutically acceptable salts thereof can be used in combination with one or more agents for the treatment of cancer. In some embodiments, the agent is an alkylating agent, a proteasome inhibitor, a corticosteroid, or an immunomodulatory agent. Examples of an alkylating agent include bendamustine, nitrogen mustards, ethylenimine derivatives, alkyl sulfonates, nitrosoureas and triazenes, uracil mustard, chlormethine, cyclophosphamide (CYTOXAN), ifosfamide, melphalan, chlorambucil, pipobroman, triethylene-melamine, triethylenethiophosphoramine, busulfan, carmustine, lomustine, streptozocin, dacarbazine, and temozolomide. In some embodiments, the proteasome inhibitor is carfilzomib. In some embodiments, the corticosteroid is dexamethasone (DEX).

The compounds of the disclosure or pharmaceutically acceptable salts thereof can be administered in combination with one or more anti-cancer drugs, such as a chemotherapeutics. Example chemotherapeutics include any of: abarelix, abiraterone, afatinib, aflibercept, aldesleukin, alemtuzumab, alitretinoin, allopurinol, altretamine, anastrozole, arsenic trioxide, asparaginase, axitinib, azacitidine, bevacizumab, bexarotene, baricitinib, bicalutamide, bleomycin, bortezombi, bortezomib, brivanib, buparlisib, busulfan intravenous, busulfan oral, calusterone, capecitabine, carmustine, cediranib, cetuximab, chlorambucil, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, docetaxel, doxorubicin, droloxafine, dromostanolone propionate, eculizumab, enzalutamide, epidophyllotoxin, epirubicin, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, nofetumomab, oserelin, paclitaxel, pamidronate, panitumumab, pazopanib, pegaspargase, pegfilgrastim, pemetrexed disodium, pentostatin, pilaralisib, pipobroman, plicamycin, cisplatin, carboplatin, oxaliplatin, ponatinib, prednisone, procarbazine, quinacrine, rasburicase, regorafenib, reloxafine, rituximab, ruxolitinib, sorafenib, streptozocin, sunitinib, sunitinib maleate, tamoxifen, tegafur, temozolomide, teniposide, testolactone, thalidomide, thioguanine, thiotepa, topotecan, toremifene, tositumomab, trastuzumab, tretinoin, triptorelin, uracil mustard, valrubicin, vandetanib, vinblastine, vincristine, vinorelbine, vorinostat, and zoledronate.

Other anti-cancer agent(s) include antibody therapeutics such as trastuzumab (Herceptin).

Compounds of the disclosure or pharmaceutically acceptable salts thereof can be administered as the sole pharmaceutical agent or in combination with one or more anti-viral agents for the treatment of chronic viral infections, where the combination causes no unacceptable adverse effects. Chronic viral infections include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV). Parasitic infections (e.g., malaria) may also be treated by the above methods wherein compounds known to treat the parasitic conditions are optionally added in place of the antiviral agents.

Suitable antiviral agents contemplated for use in combination with the compound of the disclosure or a pharmaceutically acceptable salt thereof can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-I0652; emitricitabine [(−)-FTC]; beta-L-FD4 (also called beta-L-D4C and named beta-L-2′,3′-dicleoxy-5-fluoro-cytidene); DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrirnidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

When more than one pharmaceutical agent is administered to a subject, they can be administered simultaneously, separately, sequentially, or in combination (e.g., for more than two agents). For examples, when administered together with an additional anti-cancer or antiviral agent, the disclosed compounds or pharmaceutically acceptable salts thereof can be administered simultaneously in the same pharmaceutical formulation or simultaneously in separate pharmaceutical formulations. Alternatively, when administered together with an additional anti-cancer or antiviral agent, the disclosed compounds or pharmaceutically acceptable salts thereof can be administered at separate times, depending the dosing requirements of the additional anti-cancer or antiviral agent.

Pharmaceutical compositions are disclosed that include one or more compounds provided herein (such as the compound of Formulas I, II, III, IV(A), IV(B), V, VI and VII), and typically at least one additional substance, such as an excipient, a known therapeutic other than those of the disclosure, and combinations thereof. In some embodiments, the disclosed compounds or pharmaceutically acceptable salts thereof can be used in combination with other agents known to have beneficial activity targeting diseases or disorders listed above. For example, disclosed compounds or pharmaceutically acceptable salts thereof can be administered alone or in combination with one or more anti-cancer or antiviral agent.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. A “subject” is a mammal in need of medical treatment, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The precise amount of compound or pharmaceutically acceptable salt thereof administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer or antiviral agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the disclosure or a pharmaceutically acceptable salt thereof being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

“Pharmaceutically acceptable excipient” and “pharmaceutically acceptable carrier” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

### General Synthetic Methods and Intermediates

Scheme 1 shows a synthetic protocol for the preparation of compounds of formula iii.

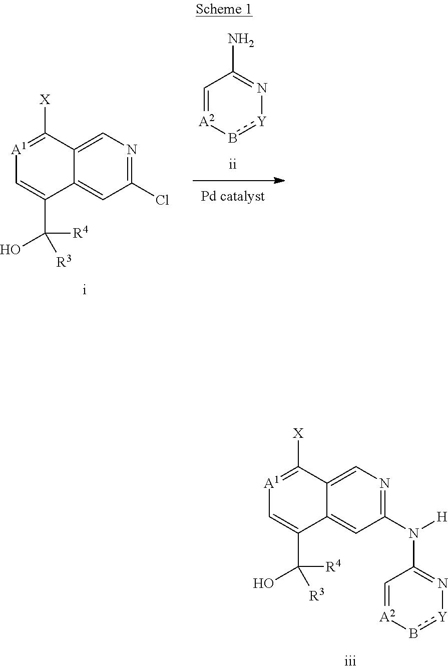

The alcohol substituted chloro heterocyclic intermediates i can be coupled to the substituted anilines ii under Pd-catalyzed coupling conditions to give compounds iii which are examples of MAP4K1 inhibitors described herein.

The following examples are intended to be illustrative and are not meant in any way to be limiting.

### EXEMPLIFICATION

### Abbreviations

ACN acetonitrile (“MeCN”)
ATP adenosine triphosphate
BOP benzotriazol-1-yloxytris(dimethylamino)phosphonium hexaflorophosphate
BrettPhos Dicyclohexyl(2′,4′,6′-triisopropyl-3,6-dimethoxy-[1,1′-biphenyl]-2-yl)phosphine
C Celsius
DAST diethylaminosulfur trifluoride
DBU diazabicyclo[5.4.0]undec-7-ene
DCM dichloromethane
DIAD disopropyl azodicarboxylate
DIPEA diisopropylethylamine
DMBNH₂ 2,4-dimethoxybenzylamine
DME dimethoxyethane
DMF dimethyl formamide
DMSO dimethyl sulfoxide
dppf [1,1′-bis(diphenylphosphino)ferrocene]dichloropalladium (II)
DTT dithiothreitol
eq equivalents
EA ethyl acetate
EDTA ethylenediaminetetracetic acid
Et₃N triethylamine
FA formic acid
h hours
HEPES 4-(2-hydroxyethyl)-1-piperazineethanesulfonic acid
HPLC high performance liquid chromatography
IBX 2-iodoxybenzoic acid
IC50 inhibitory concentration 50%
IPA isopropanol
LiHMDS lithium bis(trimethylsilyl)amide
mCPBA meta-chloroperbenzoic acid
Me₂S dimethyl sulfide
MeMgBr methyl magnesium bromide
min minutes
MTBE methyl tert-butyl ether
MeOH methanol
2-MeTHF 2-methyltetrahydrofuran
MsCl methanesulfonyl chloride
NBS N-bromosuccinimide
NMO N-methylmorpholine N-oxide
NMP N-methyl-2-pyrrolidone
Oxone® potassium peroxymonosulfate
PE petroleum ether
SFC supercritical fluid chromatography
TBAB tetrabutylammonium bromide
TBAF tetrabutylammonium fluoride
TEA trimethylamine
THF tetrahydrofuran
TFA trifluoroacetic acid
TfOH trifluoromethanesulfonic acid
TMSCF₃ trifluoromethyltrimethylsilane

Methods for preparing compounds of the disclosure can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization:

LC-MS: Unless otherwise indicated, all liquid chromatography-mass spectrometry (LC-MS) data (sample analyzed for purity and identity) were obtained with an Agilent model-1260 LC system using an Agilent model 6120 mass spectrometer utilizing ES-API ionization fitted with an Agilent Poroshel 120 (EC-C18, 2.7 um particle size, 3.0×50 mm dimensions) reverse-phase column at 22.4 degrees Celsius. The mobile phase consisted of a mixture of solvent 0.1% FA in water and 0.1% FA in acetonitrile. A constant gradient from 95% aqueous/5% organic to 5% aqueous/95% organic mobile phase over the course of 4 minutes was utilized. The flow rate was constant at 1 mL/min.

Prep LC-MS: Preparative HPLC was performed on a Shimadzu Discovery VP® Preparative system fitted with a Luna 5u C18(2) 100A, AXIA packed, 250×21.2 mm reverse-phase column at 22.4 degrees Celsius. The mobile phase consisted of a mixture of solvent 0.1% FA in water and 0.1% FA in ACN. A constant gradient from 95% aqueous/5% organic to 5% aqueous/95% organic mobile phase over the course of 25 minutes was utilized. The flow rate was constant at 20 mL/min. Reactions carried out in a microwave were done so in a Biotage Initiator microwave unit.

Silica gel chromatography: Silica gel chromatography was performed on either a Teledyne Isco CombiFlash® Rf unit or a Biotage® Isolera Four unit.

Proton NMR: Unless otherwise indicated, all ¹H NMR spectra were obtained with a Varian 400 MHz Unity Inova 400 MHz NMR instrument (acquisition time=3.5 seconds with a 1 second delay; 16 to 64 scans). Where characterized, all protons were reported in DMSO-d6 solvent as parts-per million (ppm) with respect to residual DMSO (2.50 ppm).

One of ordinary skill in the art will recognize that modifications of the gradient, column length, and flow rate are possible and that some conditions may be more suitable for compound characterization than others, depending on the chemical species being analyzed.

### Example 1: Synthesis of Arylamine Intermediates

### Example 1a

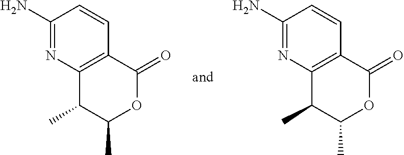

### Intermediates 1 and 2: (7S,8R)-2-Amino-7,8-dimethyl-7,8-dihydro-5H-pyrano[4,3-b]pyridin-5-one (Intermediate 1) and (7R,8S)-2-amino-7,8-dimethyl-7,8-dihydro-5H-pyrano[4,3-b]pyridin-5-one (Intermediate 2)

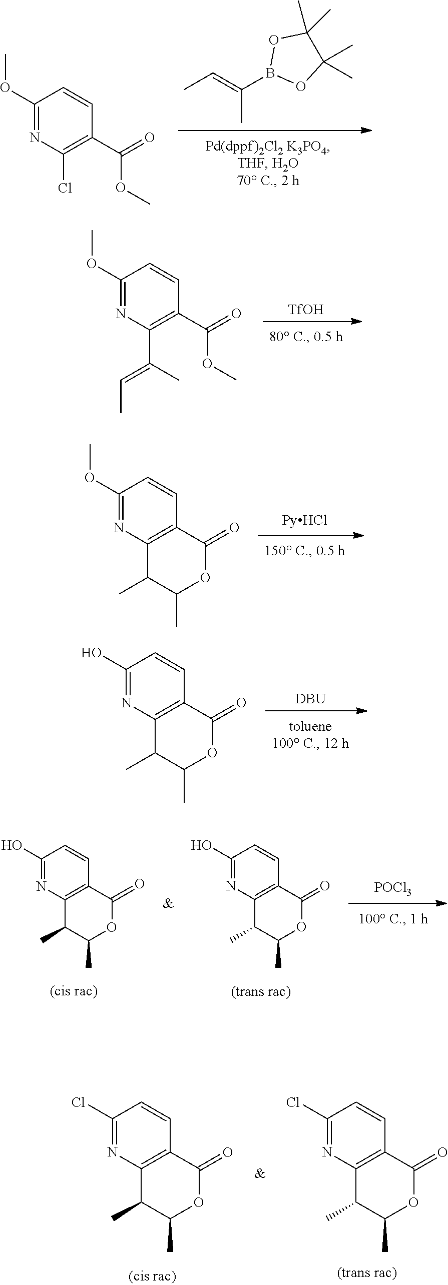 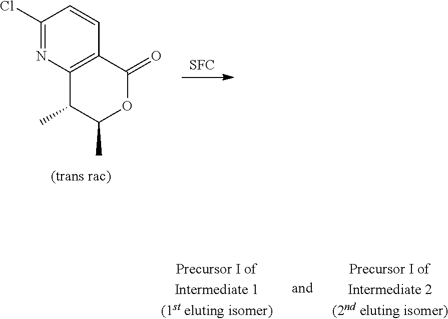 [0216] each of which is represented by the structures shown below:

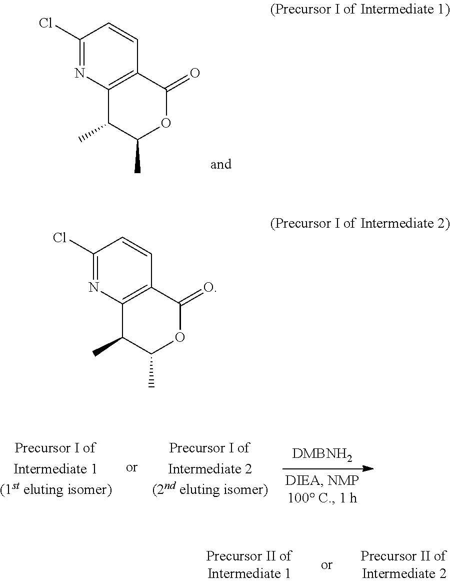

each of which is represented by the structures shown below:

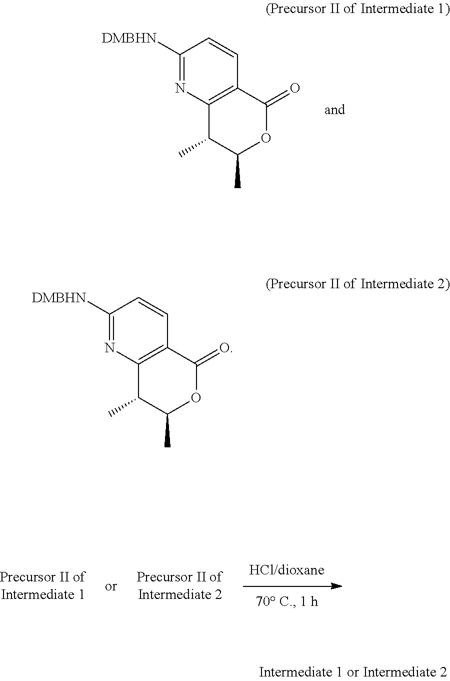

each of which is represented by the structures shown below:

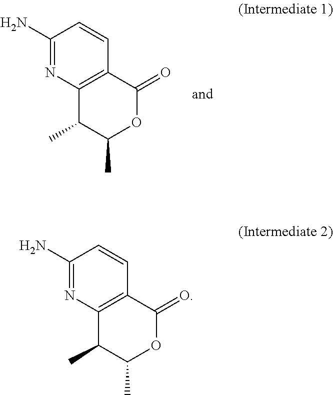

### Step 1: Methyl (E)-2-(but-2-en-2-yl)-6-methoxynicotinate

K₃PO₄ (120 g, 565 mmol, 3.00 eq) and Pd(dppf)Cl₂—CH₂Cl₂ (7.70 g, 9.42 mmol, 0.05 eq) were added to a solution of methyl 2-chloro-6-methoxynicotinate (38.0 g, 188 mmol, 1.00 eq) and (Z)-2-(but-2-en-2-yl)-4,4,5,5-tetramethyl-1,3,2-dioxaborolane (44.6 g, 245 mmol, 1.30 eq) in THF (320 mL) and H₂O (80.0 mL). The reaction mixture was stirred under N₂ at 70° C. for 2 h. The reaction mixture was diluted with water (300 mL) and extracted with EA (250 mL×3). The organic layers were combined and dried over sodium sulfate, then filtered and concentrated in vacuo to give the residue. The residue was purified by prep-HPLC (ACN-H₂O gradient with 0.1% TFA additive). The product-containing fractions were adjusted to pH=8-9 with solid sodium carbonate and the mixture was extracted with EA (300 mL×3). The combined organic layers were washed with brine (500 mL), dried over Na₂SO₄, filtered and concentrated to give the title compound (37.0 g, 167 mmol, 88.7% yield) as a yellow oil.

### Step 2: 2-Methoxy-7,8-dimethyl-7,8-dihydro-5H-pyrano[4,3-b]pyridin-5-one

A solution of methyl (E)-2-(but-2-en-2-yl)-6-methoxynicotinate (37.0 g, 167 mmol, 1.00 eq) in TfOH (171 g, 1.15 mol, 101 mL, 6.85 eq) was stirred at 80° C. for 0.5 h. The mixture was then cooled to ambient temperature, poured into saturated aqueous NaHCO₃ solution (1000 mL) and extracted with EA (300 mL×5). The organic layers were dried over sodium sulfate, filtered, and concentrated to give a residue. The residue was purified by flash-column chromatography on silica gel (gradient elution, 1% to 25% EA-PE) to give the title compound (30.0 g, 144 mmol, 86.6% yield) as a yellow oil.

### Steps 3: 2-Hydroxy-7,8-dimethyl-7,8-dihydro-5H-pyrano[4,3-b]pyridin-5-one

A mixture of 2-methoxy-7,8-dimethyl-7,8-dihydro-5H-pyrano[4,3-b]pyridin-5-one (30.0 g, 144 mmol, 1.00 eq) and pyridine-hydrochloride (41.8 g, 361 mmol, 2.50 eq) was stirred at 150° C. for 0.5 h. The reaction mixture was purified directly by flash-column chromatography on silica gel (gradient elution, 2% to 10% MeOH-DCM) to give the title compound (26.0 g, 134 mmol, 92.9% yield) as a yellow solid.

### Step 4: rac-(7S,8S)-2-Hydroxy-7,8-dimethyl-7,8-dihydro-5H-pyrano[4,3-b]pyridin-5-one and rac-(7S,8R)-2-hydroxy-7,8-dimethyl-7,8-dihydro-5H-pyrano[4,3-b]pyridin-5-one

DBU (60.8 mL, 403 mmol, 3.00 eq) was added to a solution of 2-hydroxy-7,8-dimethyl-7,8-dihydro-5H-pyrano[4,3-b]pyridin-5-one (26.0 g, 134 mmol, 1.00 eq) in toluene (290 mL). The reaction mixture was stirred at 100° C. for 12 h, then was cooled to ambient temperature and concentrated under vacuum. The residue was purified by flash-column chromatography on silica gel (gradient elution, 1% to 10% MeOH-DCM) to afford the title compounds as a mixture of isomers that were used in the next step without further purification.

### Step 5: rac-(7S,8S)-2-Chloro-7,8-dimethyl-7,8-dihydro-5H-pyrano[4,3-b]pyridin-5-one and rac-(7S,8R)-2-chloro-7,8-dimethyl-7,8-dihydro-5H-pyrano[4,3-b]pyridin-5-one

A mixture of rac-(7S,8S)-2-hydroxy-7,8-dimethyl-7,8-dihydro-5H-pyrano[4,3-b]pyridin-5-one and rac-(7S,8R)-2-hydroxy-7,8-dimethyl-7,8-dihydro-5H-pyrano[4,3-b]pyridin-5-one (26.0 g, 134 mmol, 1 equiv) in POCl₃ (150 mL, 1.61 mol, 11.9 equiv) was stirred at 100° C. for 1 h. The reaction mixture was then cooled to ambient temperature and poured into saturated aqueous NaHCO₃ solution (2 L) at 0-10° C. The quenched mixture was extracted with EA (300 mL×3) and the combined organic layers were washed with brine, dried over sodium sulfate, filtered, and concentrated. The cis- and trans-racemic isomers were separated by prep-HPLC (column: Phenomenex luna C18 250*50 mm*10 um; mobile phase: [water (0.1% TFA)—ACN]; B %: 35% ACN—55% CAN over 20 min). rac-(7S,8S)-2-Chloro-7,8-dimethyl-7,8-dihydro-5H-pyrano[4,3-b]pyridin-5-one was the first compound to elute and was obtained as a white solid. MS (ES+) C₁₀H₁₀ClNO₂ requires: 211, found: 212[M+H]⁺. ¹H NMR: 400 MHz, CDCl₃ δ 8.29 (d, J=8.2 Hz, 1H), 7.39 (d, J=8.2 Hz, 1H), 4.83 (dq, J=3.2, 6.6 Hz, 1H), 3.09 (dq, J=3.2, 7.2 Hz, 1H), 1.49 (d, J=6.5 Hz, 3H), 1.30 (d, J=7.2 Hz, 3H). rac-(7S,8R)-2-Chloro-7,8-dimethyl-7,8-dihydro-5H-pyrano[4,3-b]pyridin-5-one was the second compound to elute and was obtained as a white solid. MS (ES+) C₁₀H₁₀ClNO₂ requires: 211, found: 212[M+H]⁺. ¹H NMR: 400 MHz, CDCl₃ δ 8.28 (d, J=8.2 Hz, 1H), 7.38 (d, J=8.2 Hz, 1H), 4.58-4.43 (m, 1H), 3.05 (quin, J=7.2 Hz, 1H), 1.56-1.40 (m, 6H).

### Step 6: (7S,8R)-2-Chloro-7,8-dimethyl-7,8-dihydro-5H-pyrano[4,3-b]pyridin-5-one and (7R,8S)-2-chloro-7,8-dimethyl-7,8-dihydro-5H-pyrano[4,3-b]pyridin-5-one

rac-(7S,8R)-2-Chloro-7,8-dimethyl-7,8-dihydro-5H-pyrano[4,3-b]pyridin-5-one was separated by SFC (column: DAICEL CHIRALPAK AD (250 mm×50 mm, 10 um); mobile phase: MeOH in CO₂) to give the first eluting isomer (peak 1) as a white solid and second eluting isomer (peak 2) as a white solid.

### Step 7: (7S,8R)-2-((2,4-Dimethoxybenzyl)amino)-7,8-dimethyl-7,8-dihydro-5H-pyrano[4,3-b]pyridin-5-one

DIPEA (7.48 g, 57.8 mmol, 10.1 mL, 2.50 eq) and DMBNH₂ (5.03 g, 30.1 mmol, 4.53 mL, 1.30 eq) were added to a solution of (7S,8R)-2-chloro-7,8-dimethyl-7,8-dihydro-5H-pyrano[4,3-b]pyridin-5-one (1^{st} eluting isomer (peak 1) from Step 6 above) (4.90 g, 23.1 mmol, 1.00 eq) in NMP (50.0 mL). The reaction mixture was stirred at 100° C. for 1 h, then was poured into water (500 mL) and extracted with EA (100 mL×3). The combined organic layers were washed with brine (200 mL), dried over sodium sulfate, filtered and concentrated to give the title compound (7.93 g, crude) as a yellow oil that was used directly in the next step. MS (ES+) C₁₉H₁₁N₂O₄ requires: 342, found: 343[M+H]⁺.

### Step 8: (7S,8R)-2-Amino-7,8-dimethyl-7,8-dihydro-5H-pyrano[4,3-b]pyridin-5-one

A solution of (7S,8R)-2-((2,4-Dimethoxybenzyl)amino)-7,8-dimethyl-7,8-dihydro-5H-pyrano[4,3-b]pyridin-5-one (7.93 g, 23.1 mmol, 1.00 eq) in HCl/dioxane (4.00 M, 50.0 mL, 8.64 eq) was stirred at 70° C. for 1 h. The reaction mixture was then concentrated and partitioned between saturated aqueous sodium bicarbonate solution (100 mL) and was extracted with DCM (100 mL×3). The combined organic layer was washed with brine (200 mL), dried over sodium sulfate, filtered and concentrated. The residue was triturated in MTBE (50 mL) for 10 mins and filtered to get a yellow solid. The yellow solid was dried under vacuum to give the title compound, Intermediate 1(3.23 g, 16.4 mmol, 71.2% yield, 98.1% purity). MS (ES+) C₁₀H₁₂N₂O₂ requires: 192, found: 193[M+H]⁺. ¹H NMR: 400 MHz, DMSO-d6 δ 7.77 (d, J=8.6 Hz, 1H), 6.97 (s, 2H), 6.40 (d, J=8.6 Hz, 1H), 4.43-4.21 (m, 1H), 2.88-2.65 (m, 1H), 1.35 (d, J=6.4 Hz, 3H), 1.25 (d, J=7.0 Hz, 3H). The absolute stereochemistry of Intermediate 1 was determined by X-ray crystal structure.

### Steps 9 and 10: (7R,8S)-2-Amino-7,8-dimethyl-7,8-dihydro-5H-pyrano[4,3-b]pyridin-5-one

The title compound (Intermediate 2) was prepared separately from (7R,8S)-2-chloro-7,8-dimethyl-7,8-dihydro-5H-pyrano[4,3-b]pyridin-5-one (the second eluting isomer (peak 2) of Step 6) using the same two-step procedure as described in Steps 7 and 8 for Intermediate 2. MS (ES+) C₁₀H₁₂N₂O₂ requires: 192, found: 193[M+H]⁺. ¹H NMR: 400 MHz, DMSO-d₆ δ 7.77 (d, J=8.6 Hz, 1H), 6.97 (s, 2H), 6.40 (d, J=8.6 Hz, 1H), 4.43-4.21 (m, 1H), 2.88-2.65 (m, 1H), 1.35 (d, J=6.4 Hz, 3H), 1.25 (d, J=7.0 Hz, 3H).

### Example 1b

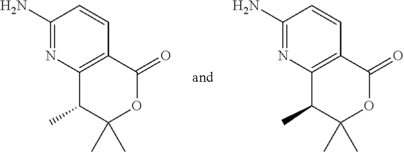

### Intermediates 3 and 4: (R)-2-amino-7,7,8-trimethyl-7,8-dihydro-5H-pyrano[4,3-b]pyridin-5-one and (S)-2-amino-7,7,8-trimethyl-7,8-dihydro-5H-pyrano[4,3-b]pyridin-5-one

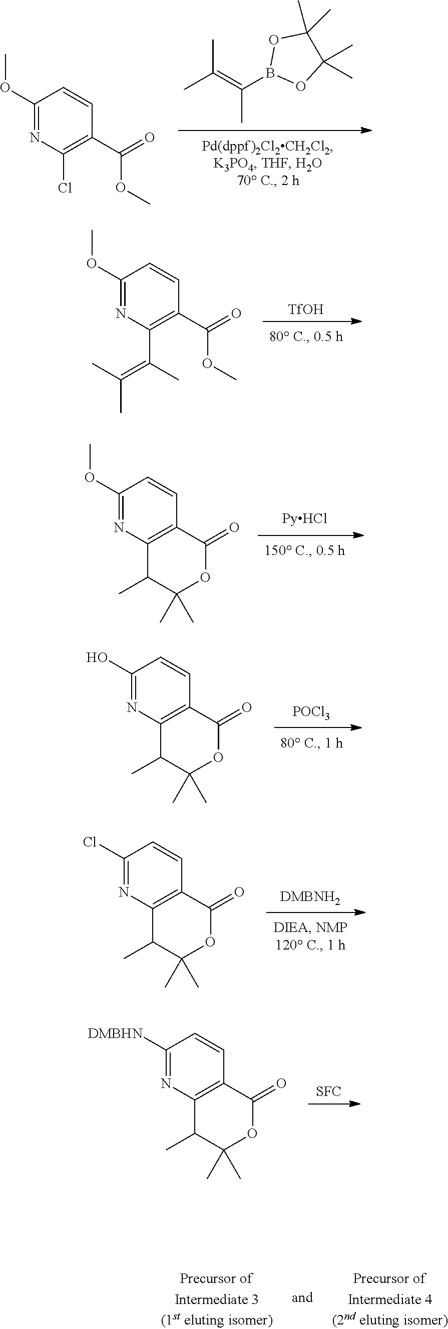 [0230] each of which is represented by the structure shown below:

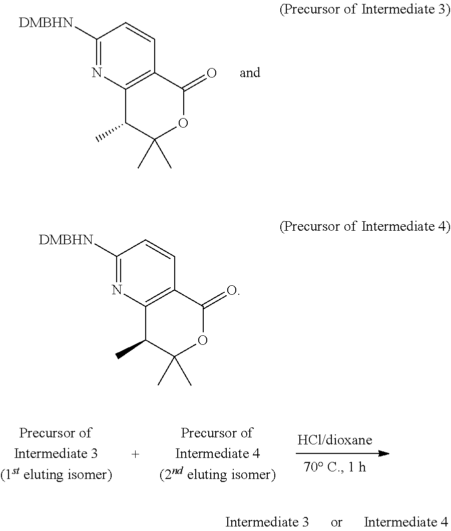

each of which is represented by the structure shown below:

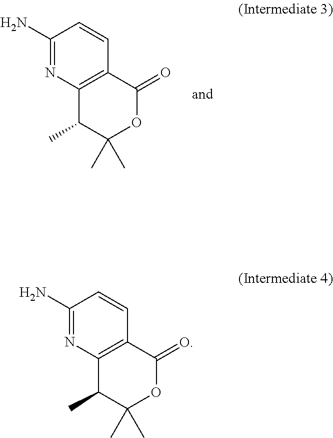

### Steps 1-5: rac 2-((2,4-Dimethoxybenzyl)amino)-7,7,8-trimethyl-7,8-dihydro-5H-pyrano[4,3-b]pyridin-5-one

The title compound was prepared from methyl 2-chloro-6-methoxynicotinate and 4,4,5,5-tetramethyl-2-(3-methylbut-2-en-2-yl)-1,3,2-dioxaborolane using similar procedures as described above in Steps 1-3 and 5-7 for Intermediate 1.

### Step 6: (R)-2-((2,4-Dimethoxybenzyl)amino)-7,7,8-trimethyl-7,8-dihydro-5H-pyrano[4,3-b]pyridin-5-one and (S)-2-((2,4-Dimethoxybenzyl)amino)-7,7,8-trimethyl-7,8-dihydro-5H-pyrano[4,3-b]pyridin-5-one

Rac 2-((2,4-Dimethoxybenzyl)amino)-7,7,8-trimethyl-7,8-dihydro-5H-pyrano[4,3-b]pyridin-5-one was separated by SFC (column: DAICEL CHIRALPAK AS-H (250 mm*30 mm, 5 um); mobile phase: [0.1% NH₄OH MeOH in CO₂]) to give (R)-2-((2,4-dimethoxybenzyl)amino)-7,7,8-trimethyl-7,8-dihydro-5H-pyrano[4,3-b]pyridin-5-one (1^{st} eluting isomer (Precursor to Intermediate 3), 0.55 g, 79% yield) and (S)-2-((2,4-dimethoxybenzyl)amino)-7,7,8-trimethyl-7,8-dihydro-5H-pyrano[4,3-b]pyridin-5-one (2^{nd} eluting isomer (Precursor to Intermediate 4), 0.55 g, 79% yield). Each intermediate was isolated as a yellow oil.

### Step 7 and 8: (R)-2-amino-7,7,8-trimethyl-7,8-dihydro-5H-pyrano[4,3-b]pyridin-5-one and (S)-2-amino-7,7,8-trimethyl-7,8-dihydro-5H-pyrano[4,3-b]pyridin-5-one

The title compounds (Intermediates 3 and 4) were prepared separately from the 1^{st} and 2^{nd} eluting isomers, i.e., (R)-2-((2,4-dimethoxybenzyl)amino)-7,7,8-trimethyl-7,8-dihydro-5H-pyrano[4,3-b]pyridin-5-one and (S)-2-((2,4-Dimethoxybenzyl)amino)-7,7,8-trimethyl-7,8-dihydro-5H-pyrano[4,3-b]pyridin-5-one, using the same procedure as described in Step 8 of Intermediate 1. Intermediate 3, (R)-2-amino-7,7,8-trimethyl-7,8-dihydro-5H-pyrano[4,3-b]pyridin-5-one, was obtained as a yellow solid. MS (ES+) C₁₁H₁₄N₂O₂ requires: 206, found: 207[M+H]⁺. ¹H-NMR (400 MHz, CD₃OD): δ ppm 7.89 (d, J=8.8 Hz, 1H), 6.50 (d, J=8.8 Hz, 1H), 2.85-2.80 (m, 1H), 1.41 (s, 6H), 1.27 (d, J=7.2 Hz, 3H). Intermediate 4, (S)-2-Amino-7,7,8-trimethyl-7,8-dihydro-5H-pyrano[4,3-b]pyridin-5-one, was obtained as a yellow solid. MS (ES+) C₁₁H₁₄N₂O₂ requires: 206, found: 207[M+H]⁺. ¹H-NMR (400 MHz, CD₃OD): δ ppm 7.89 (d, J=8.8 Hz, 1H), 6.50 (d, J=8.8 Hz, 1H), 2.85-2.80 (m, 1H), 1.41 (s, 6H), 1.27 (d, J=7.2 Hz, 3H). The stereochemistry of Intermediate 3 was determined in the context of compound 16 using an X-ray crystal structure.

### Example 1c

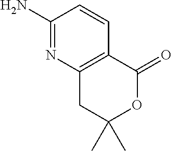

### Intermediate 5: 2-Amino-7,7-dimethyl-7,8-dihydro-5H-pyrano[4,3-b]pyridin-5-one

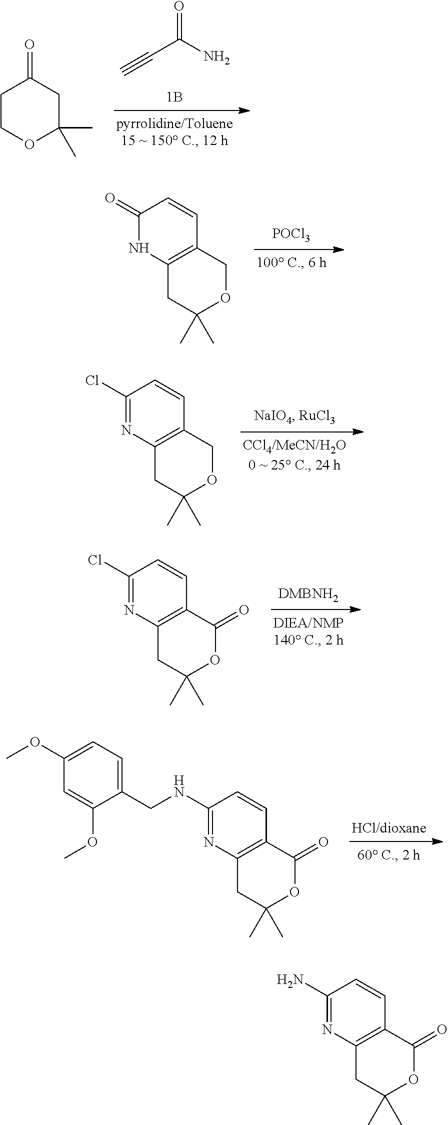

### Step 1: 7,7-Dimethyl-1,5,7,8-tetrahydro-2H-pyrano[4,3-b]pyridin-2-one

A mixture of 2,2-dimethyltetrahydro-4H-pyran-4-one (500 g, 3.90 mol, 1.00 eq) and pyrrolidine (391 mL, 4.68 mol, 1.20 eq) in toluene (4.00 L) was heated at 145° C. with a Dean-Stark trap for 2 h. The water layer (˜16 mL) was removed from the Dean-Stark trap and the reaction mixture was cooled to 15° C. After cooling, prop-2-ynamide (539 g, 7.80 mol, 2.00 eq) was added and the reaction mixture was heated to 150° C. The reaction mixture was heated at 150° C. for 10 h, then was cooled to ambient temperature. The cooled reaction mixture was filtered, and the filtrate was concentrated under reduced pressure to give a residue. The residue was purified by flash-column chromatography on silica gel (10% methanol-dichloromethane) to give the title compound (560 g, 62% yield) as a yellow solid.

### Step 2: 2-Chloro-7,7-dimethyl-7,8-dihydro-5H-pyrano[4,3-b]pyridine

A solution of 7,7-dimethyl-1,5,7,8-tetrahydro-2H-pyrano[4,3-b]pyridin-2-one (500 g, 2.23 mol, 1 eq) in POCl₃ (350 mL, 3.77 mol, 9.64 eq) was heated to 100° C. for 6 h. The reaction mixture then cooled to ambient temperature and concentrated under vacuum. The residue was poured over ice-water (1.00 L). The mixture was extracted with EA (750 mL×2). The combined organic layers were dried over Na₂SO₄, filtered and concentrated under vacuum to give the title compound (363 g, 82.2% yield) as a brown oil.

### Step 3: 2-Chloro-7,7-dimethyl-7,8-dihydro-5H-pyrano[4,3-b]pyridin-5-one

A solution of NaIO₄ (487 g, 2.28 mol, 3.00 eq) in water (1.20 L) as added to a mixture of 2-chloro-7,7-dimethyl-7,8-dihydro-5H-pyrano[4,3-b]pyridine (150 g, 759 mmol, 1.00 eq) in MeCN (50.0 mL) and CCl₄ (2.70 L). The mixture was cooled to 0° C., and then RuCl₃ (11.0 g, 53.1 mmol, 0.07 eq) was added. The reaction mixture was stirred at 0° C. for 0.5 h, then was warmed to 20° C. for 11.5 h. Saturated aqueous sodium sulfite solution (1.00 L) was added, and the mixture was filtered. The filtrate was extracted with EA (500 mL×3), and the organic layers were combined. The combined organic layer was washed with brine (1.00 L), dried over Na₂SO₄, filtered and concentrated to give the title compound (132 g, 624 mmol, 82.1% yield) as a yellow solid.

### Step 4: 2-((2,4-Dimethoxybenzyl)amino)-7,7-dimethyl-7,8-dihydro-5H-pyrano[4,3-b]pyridin-5-one

(2,4-Dimethoxyphenyl) methanamine (160 g, 957 mmol, 1.50 eq) was added to a solution of 2-chloro-7,7-dimethyl-7,8-dihydro-5H-pyrano[4,3-b]pyridin-5-one (135 g, 638 mmol, 1.00 eq) and DIPEA (222 mL, 1.28 mol, 2.00 eq) in NMP (1.08 L) at ambient temperature. The reaction mixture was heated to 140° C. for 2 h, and then was cooled to ambient temperature. The reaction mixture was then partitioned between water (700 mL) and EA. The layers were separated, and the aqueous layer was further extracted with EA (500 mL×3). The organic layers were combined and washed with brine (400 mL×3), dried over anhydrous sodium sulfate, filtered and concentrated to afford the title compound as a yellow solid (160 g). The crude product was used for next step directly.

### Step 5: 2-Amino-7,7-dimethyl-7,8-dihydro-5H-pyrano[4,3-b]pyridin-5-one

HCl (4.0 M in dioxane, 1.20 L, 11.0 equiv) was added to: 2-((2,4-dimethoxybenzyl)amino)-7,7-dimethyl-7,8-dihydro-5H-pyrano[4,3-b]pyridin-5-one (150 g, 438 mmol, 1.00 eq) at 20° C. The reaction mixture was heated to 60° C. for 2 h, then was cooled to ambient temperature and concentrated under vacuum. The residue was poured into saturated NaHCO₃ aqueous solution (1.00 L) and extracted with EA (500 mL×4). The combined organic layer was washed with brine (500×2), dried over Na₂SO₄, filtered and concentrated. The residue was dissolved in EA (300 mL) and petroleum ether (150 mL) was added drop wise to get yellow slurry. The solids were filtered and collected to give the title compound (52.0 g, 60.9% yield) as a yellow solid. MS (ES+) C₁₀H₁₂N₂O₂ requires: 192, found: 193[M+H]⁺. ¹H NMR (400 MHz, DMSO-d6) δ 7.76 (d, J=8.4 Hz, 1H), 6.98 (s, 2H), 6.39 (d, J=8.8 Hz, 1H), 2.89 (s, 2H), 1.37 (s, 6H).

### Example 1d

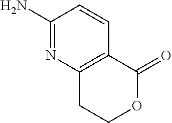 [SMILES: Nc1ccc2c(n1)CCOC2=O]

### Intermediate 6: 2-Amino-7,8-dihydro-5H-pyrano[4,3-b]pyridin-5-one

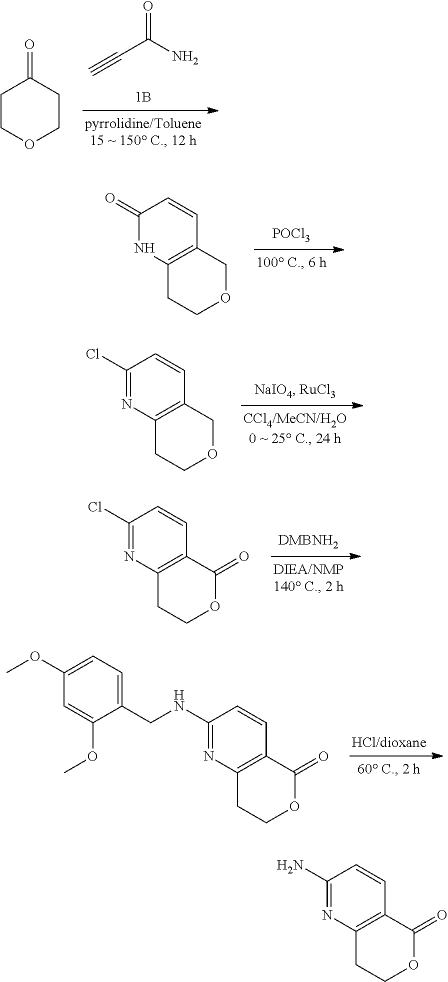

### Step 1: 2-Amino-7,8-dihydro-5H-pyrano[4,3-b]pyridin-5-one

The title compound was prepared from tetrahydro-4H-pyran-4-one using the same five-step procedure described in Steps 1-5 for Intermediate 5. MS (ES+) C₈H₈N₂O₂ requires: 164, found: 165[M+H]⁺. ¹H NMR, 400 MHz, DMSO-d6, δ=7.77 (d, J=8.8 Hz, 1H), 7.01 (s, 2H), 6.41 (d, J=8.8 Hz, 1H), 4.44-4.41 (m, 2H), 2.88-2.85 (m, 2H).

### Example 1e

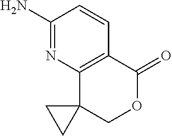 [SMILES: Nc1ccc2c(n1)C1(CC1)COC2=O]

### Intermediate 7: 2′-Amino-5′H,7′H-spiro[cyclopropane-1,8′-pyrano[4,3-b]pyridin]-5′-one

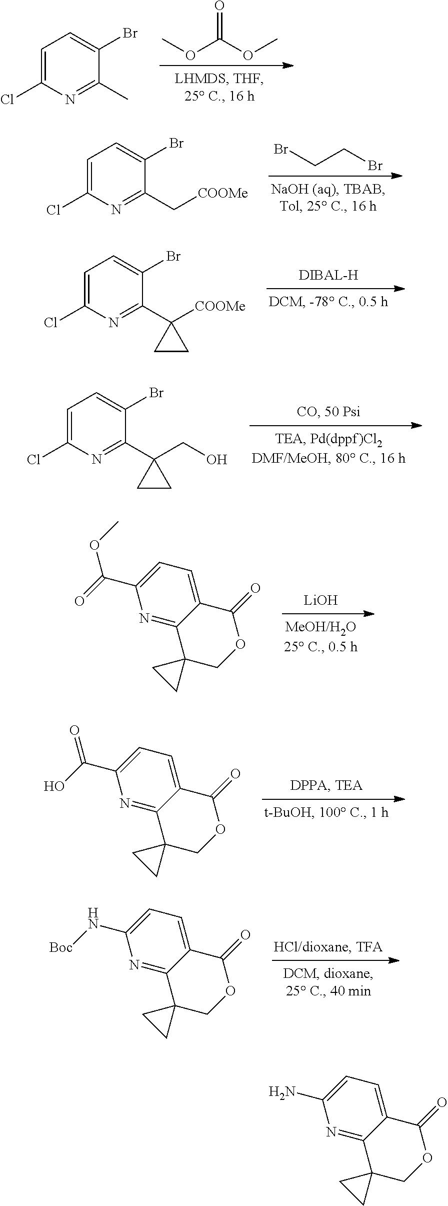

### Step 1: Methyl 2-(3-bromo-6-chloropyridin-2-yl)acetate

LiHMDS (1 M, 388 mL) was added to a solution of 3-bromo-6-chloro-2-methylpyridine (20.0 g, 96.9 mmol) in THF (300 mL) at 25° C. under nitrogen. After 2.5 h, dimethyl carbonate (14.0 g, 155 mmol) was added to the mixture and stirred at 25° C. for 13.5 h. The reaction mixture was then was added to saturated aqueous NH₄Cl (1000 mL) and extracted with EA (60 mL×3). The combined organic layers were dried over anhydrous sodium sulfate, filtered and concentrated to give a residue. The residue was purified by flash-column chromatography on silica gel (gradient elution, 0% to 10% EA-petroleum ether) to give the title compound (18.0 g, 70% yield) as a yellow oil. ¹H NMR (400 MHz, CDCl₃): δ ppm 7.81 (d, J=8.4 Hz, 1H), 7.16 (d, J=8.0 Hz, 1H), 4.03 9s, 2H), 3.74 (s, 3H).

### Step 2: Methyl 1-(3-bromo-6-chloropyridin-2-yl)cyclopropane-1-carboxylate

Tetrabutylammonium bromide (2.44 g, 7.56 mmol) and NaOH (50 mL, 50 wt % in water) were added to a solution of 1,2-dibromoethane (10.7 g, 56.7 mmol) and methyl 2-(3-bromo-6-chloropyridin-2-yl)acetate (10.0 g, 37.8 mmol) in toluene (50 mL) at 25° C. The reaction mixture was stirred at 25° C. for 16 h, then was diluted with water (300 mL) and extracted with EA (200 mL×3). The combined organic layers were dried over anhydrous sodium sulfate, filtered and concentrated to give a residue. The residue was purified by flash-column chromatography on silica gel (gradient elution, 0% to 10% EA-petroleum ether) to give the title compound (6.10 g, 56% yield) as a yellow solid. ¹H NMR (400 MHz, CDCl₃): δ ppm 7.81 (d, J=8.4 Hz, 1H), 7.15 (d, J=8.0 Hz, 1H), 3.66 (s, 3H), 1.81-1.75 (m, 2H), 1.46-1.41 (m, 2H).

### Step 3: (1-(3-Bromo-6-chloropyridin-2-yl)cyclopropyl)methanol

Diisobutylaluminium hydride (1 M, 56 mL) was added to a solution of methyl 1-(3-bromo-6-chloropyridin-2-yl)cyclopropane-1-carboxylate (5.40 g, 18.6 mmol) in DCM (80 mL) at −78° C. under nitrogen. The reaction mixture was stirred at −78° C. for 0.5 h, then was quenched by addition of aqueous saturated NH₄Cl solution (50 mL), diluted with water (200 mL) and extracted with EA (200 mL×3). The combined organic layers were dried over anhydrous sodium sulfate, filtered and concentrated to give the crude title compound (5.00 g, crude) as a yellow solid which was used in the next step without further purification.

### Step 4: Methyl 5′-oxo-5′H,7′H-spiro[cyclopropane-1,8′-pyrano[4,3-b]pyridine]-2′-carboxylate

Triethylamine (2.31 g, 22.9 mmol) and Pd(dppf)Cl₂ (557 mg, 762 μmol) were added to a solution of (1-(3-Bromo-6-chloropyridin-2-yl)cyclopropyl)methanol in MeOH (25 mL) and DMF (25 mL) under nitrogen atmosphere. The suspension was degassed under vacuum and purged with carbon monoxide several times. The mixture was stirred under carbon monoxide (50 psi) at 80° C. for 16 h. The reaction mixture was then concentrated to remove methanol, diluted with water (100 mL) and extracted with EA (60 mL×3). The combined organic layers were washed with brine (100 mL×3), dried over anhydrous sodium sulfate, filtered and concentrated to give a residue. The title compound (1.8 g, crude) was obtained as a yellow solid and used in the next step without further purification. MS (ES+) C₁₀H₁₀N₂O₂ requires: 233, found: 234[M+H]⁺.

### Step 5: 5′-Oxo-5′H,7′H-spiro[cyclopropane-1,8′-pyrano[4,3-b]pyridine]-2′-carboxylic acid

Lithium hydroxide (555 mg, 23.2 mmol) was added to a solution of methyl 5′-oxo-5′H,7′H-spiro[cyclopropane-1,8′-pyrano[4,3-b]pyridine]-2′-carboxylate (1.80 g, 7.72 mmol) in methanol (30 mL) and water (10 mL). The reaction mixture was stirred at 25° C. for 0.5 h, then was concentrated to remove the methanol. The mixture was diluted with water (60 mL) and extracted with EA (50 mL×3). The aqueous layer was acidified by addition aqueous hydrochloric acid solution (6 M, 5 mL), then the mixture was extracted with EA (50 mL×3). The combined organic layers were dried over anhydrous sodium sulfate, filtered and concentrated to give the title compound (1.20 g, 71% yield) as a brown solid that was used without further purification.

### Step 6: tert-Butyl (5′-oxo-5′H,7′H-spiro[cyclopropane-1,8′-pyrano[4,3-b]pyridin]-2′-yl)carbamate

Triethylamine (831 mg, 8.21 mmol) and diphenyl phosphoryl azide (2.26 g, 8.21 mmol) were added to a solution of 5′-oxo-5′H,7′H-spiro[cyclopropane-1,8′-pyrano[4,3-b]pyridine]-2′-carboxylic acid (1.20 g, 5.47 mmol) in tert-butanol (20 mL). The reaction mixture was stirred at 100° C. for 1 h, then was cooled to ambient temperature, diluted with water (60 mL), and extracted with EA (50 mL×3). The combined organic layers were dried over anhydrous sodium sulfate, filtered and concentrated to give a residue. The residue was purified by flash-column chromatography on silica gel (gradient elution, 0% to 50% EA-petroleum ether) to give the title compound (330 mg, 19% yield) as a yellow solid and 2′-amino-5′H,7′H-spiro[cyclopropane-1,8′-pyrano[4,3-b]pyridin]-5′-one (420 mg, 28% yield) as a yellow oil.

### Step 7: 2′-Amino-5′H,7′H-spiro[cyclopropane-1,8′-pyrano[4,3-b]pyridin]-5′-one

HCl in dioxane (4.0 M, 0.5 mL) was added to a solution of tert-butyl (5′-oxo-5′H,7′H-spiro[cyclopropane-1,8′-pyrano[4,3-b]pyridin]-2′-yl)carbamate (100 mg, 344 μmol) in dioxane (1.5 mL) at 25° C. The reaction mixture was stirred for 10 min, then was concentrated. DCM (2 mL) and TFA (1 mL, 13.5 mmol) were added to the residue, and the reaction mixture was stirred at 25° C. for 30 min. The reaction mixture was then concentrated and EA (5 mL) was added to the residue. The mixture was neutralized by addition of saturated aqueous NaHCO₃(20 mL) and extracted with EA (15 mL×3). The combined organic layers were dried over anhydrous sodium sulfate, filtered and concentrated under to give the title compound (60.0 mg, 92% yield) as a yellow oil that was used in the next step without further purification. MS (ES+) C₁₂H₁₁NO₄ requires: 190, found: 191[M+H]⁺.

### Example 1f

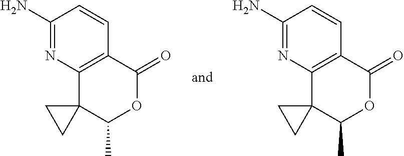

### Intermediates 8 and 9: (R)-2′-Amino-7′-methyl-5′H,7′H-spiro[cyclopropane-1,8′-pyrano[4,3-b]pyridin]-5′-one and (S)-2′-Amino-7′-methyl-5′H,7′H-spiro[cyclopropane-1,8′-pyrano[4,3-b]pyridin]-5′-one

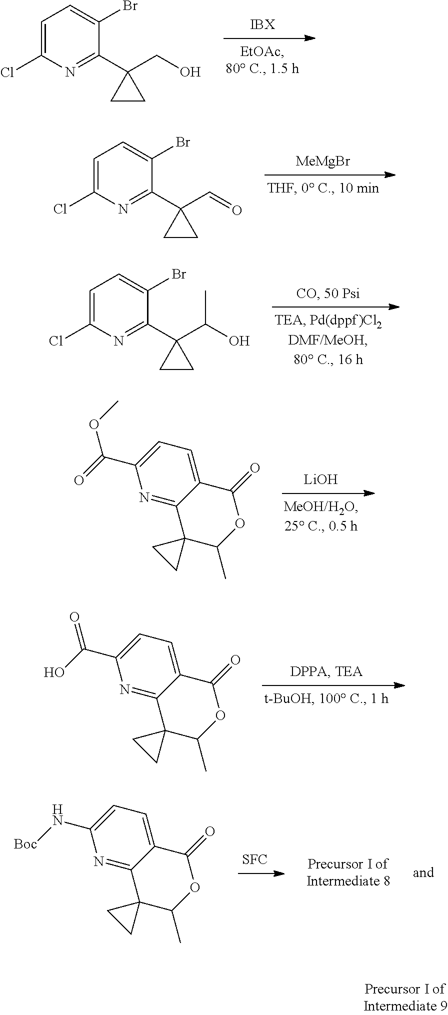 [0256] each of which is represented by one of the structures shown below:

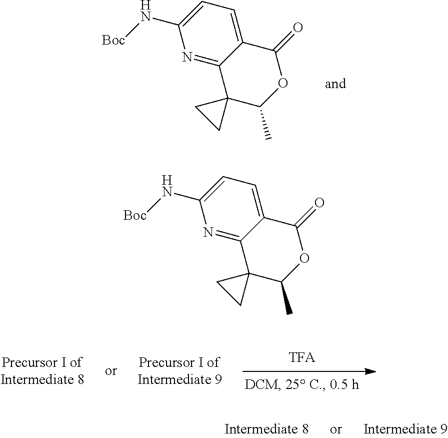

which is represented by one of the structures shown below:

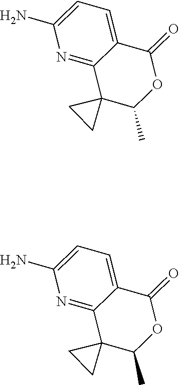

### Step 1: 1-(3-Bromo-6-chloropyridin-2-yl)cyclopropane-1-carbaldehyde

IBX (6.50 g, 10.7 mmol, 46% purity) was added to a solution of (1-(3-bromo-6-chloropyridin-2-yl)cyclopropyl)methanol (2.65 g, 10.1 mmol) in EA (80 mL). The reaction mixture was stirred at 80° C. for 1 h, then additional IBX (2.00 g, 3.29 mmol, 46% purity) was added. The reaction mixture was stirred at 80° C. for 0.5 h, then was filtered and concentrated to give the title compound (2.60 g, crude) as a yellow solid that was used without further purification. MS (ES+) C₉H₇BrClNO requires: 261, found: 262 [M+H]⁺.

### Step 2: 1-(1-(3-Bromo-6-chloropyridin-2-yl)cyclopropyl)ethan-1-ol

Methylmagnesium bromide (3 M, 17 mL) was added to a solution of 1-(3-bromo-6-chloropyridin-2-yl)cyclopropane-1-carbaldehyde (2.60 g, 9.98 mmol) in THF (80 mL) at 0° C. The reaction mixture was stirred for 10 min, then was quenched by addition of aqueous saturated NH₄Cl solution (80 mL), diluted with water (40 mL) and extracted with EA (80 mL×3). The combined organic layers were dried over anhydrous sodium sulfate, filtered and concentrated to give the title compound (2.70 g, crude) as a yellow oil that was used without further purification. MS (ES+) C₁₀H₁₁N₂O₂ requires: 277, found: 278 [M+H]⁺.

### Steps 3-5: tert-Butyl (7′-methyl-5′-oxo-5′H,7′H-spiro[cyclopropane-1,8′-pyrano[4,3-b]pyridin]-2′-yl)carbamate

The title compound was prepared from 1-(1-(3-bromo-6-chloropyridin-2-yl)cyclopropyl)ethan-1-ol using a similar procedure as described in Steps 4-6 for Intermediate 7 above. ¹H NMR (400 MHz, CDCl₃): δ ppm 8.29 (d, J=8.8 Hz, 1H), 7.90 (d, J=8.8 Hz, 1H), 7.31 (s, 1H), 4.63-4.53 (m, 1H), 1.61 (s, 3H), 1.53 (s, 9H), 1.38-1.35 (m, 1H), 1.09-1.00 (m, 2H).

### Step 6: tert-Butyl (R)-(7′-methyl-5′-oxo-5′H,7′H-spiro[cyclopropane-1,8′-pyrano[4,3-b]pyridin]-2′-yl)carbamate and tert-butyl (S)-(7′-methyl-5′-oxo-5′H,7′H-spiro[cyclopropane-1,8′-pyrano[4,3-b]pyridin]-2′-yl)carbamate

tert-Butyl (7′-methyl-5′-oxo-5′H,7′H-spiro[cyclopropane-1,8′-pyrano[4,3-b]pyridin]-2′-yl)carbamate (400 mg) was separated by SFC (column: REGIS (s,s) WHELK-O1 (250 mm×50 mm, 10 um), EtOH gradient in CO₂ with 0.1% NH₄OH) to give two separate peaks. The first eluting isomer (100 mg, 24% yield) and second eluting isomer (140 mg, 34% yield) were obtained as yellow solids.

### Step 7: One of (R or S)-2′-amino-7′-methyl-5′H,7′H-spiro[cyclopropane-1,8′-pyrano[4,3-b]pyridin]-5′-one

TFA (2.31 g, 20.3 mmol) was added to a solution of one of tert-butyl (R or S)-(7′-methyl-5′-oxo-5′H,7′H-spiro[cyclopropane-1,8′-pyrano[4,3-b]pyridin]-2′-yl)carbamate (first eluting isomer from Step 6, 100 mg) in DCM (6 mL). The reaction mixture was stirred at 25° C. for 30 min, then was quenched with saturated aqueous NaHCO₃ solution (30 mL) and extracted with DCM (20 mL×3). The combined organic layers were dried over anhydrous sodium sulfate, filtered and concentrated to give the title compound (Intermediate 8, 70 mg, crude) as a yellow oil that was used without further purification. MS (ES+) C₁₁H₁₂N₂O₂ requires: 204, found: 205[M+H]⁺.

### Step 8: The remaining one of (R or S)-2′-amino-7′-methyl-5′H,7′H-spiro[cyclopropane-1,8′-pyrano[4,3-b]pyridin]-5′-one

The title compound (Intermediate 9) was prepared from one of tert-butyl (R or S)-(7′-methyl-5′-oxo-5′H,7′H-spiro[cyclopropane-1,8′-pyrano[4,3-b]pyridin]-2′-yl)carbamate (second eluting isomer from Step 6) using the same procedure as described in Step 7 for Intermediate 8. MS (ES+) C₁₁H12N₂₀₂ requires: 204, found: 205[M+H]⁺.

### Example 1g

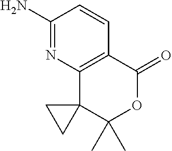 [SMILES: CC1(C)OC(=O)c2ccc(N)nc2C12CC2]

### Intermediate 10: 2′-Amino-7′,7′-dimethyl-5′H,7′H-spiro[cyclopropane-1,8′-pyrano[4,3-b]pyridin]-5′-one

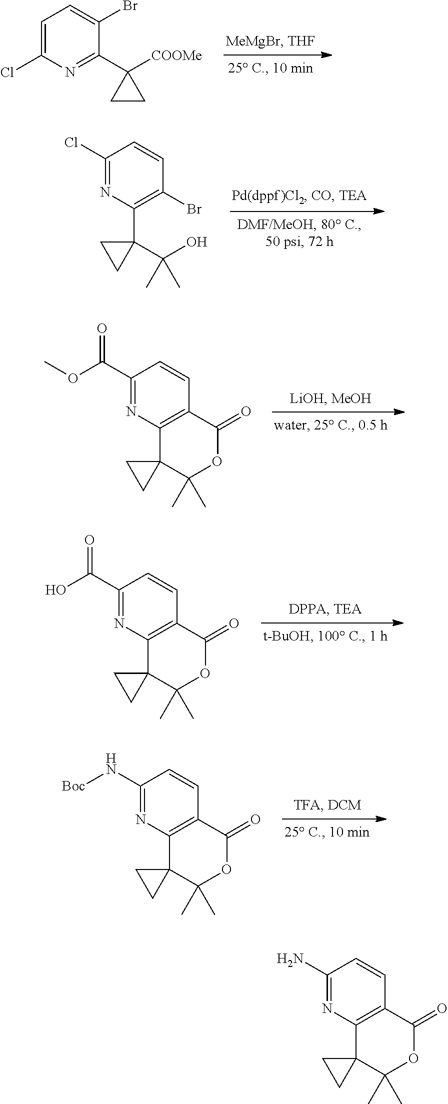

### Step 1: 2-(1-(3-Bromo-6-chloropyridin-2-yl)cyclopropyl)propan-2-ol

To a solution of methyl 1-(3-bromo-6-chloropyridin-2-yl)cyclopropane-1-carboxylate (1.3 g, 4.47 mmol) in THF (10 mL) was added MeMgBr (3 M, 14.9 mL) at 25° C. The reaction mixture was stirred at 25° C. for 10 min, then was poured into water (20 mL) and extracted with EA (50 mL×3). The organic layers were combined and dried over sodium sulfate, filtered, and concentrated to give a residue. The residue was purified by column chromatography (SiO₂, PE/EA=1/0 to 50/1) to give the title compound (500 mg, 38% yield) as colorless oil.

### Steps 2-5: 2′-Amino-7′,7′-dimethyl-5′H,7′H-spiro[cyclopropane-1,8′-pyrano[4,3-b]pyridin]-5′-one

The title compound was prepared from 2-(1-(3-Bromo-6-chloropyridin-2-yl)cyclopropyl)propan-2-ol using a similar procedure as described in Steps 4-6 for Intermediate 7 and Step 7 of Intermediate 8 above. ¹H NMR (400 MHz, CDCl₃): δ ppm 8.05 (d, J=8.4 Hz, 1H), 6.36 (d, J=8.8 Hz, 1H), 4.85 (s, 2H), 1.42-1.32 (m, 8H), 1.06-1.03 (m, 2H).

### Example 1h

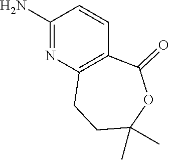

### Intermediate 11: 2-Amino-7,7-dimethyl-8,9-dihydrooxepino[4,3-b]pyridin-5(7H)-one

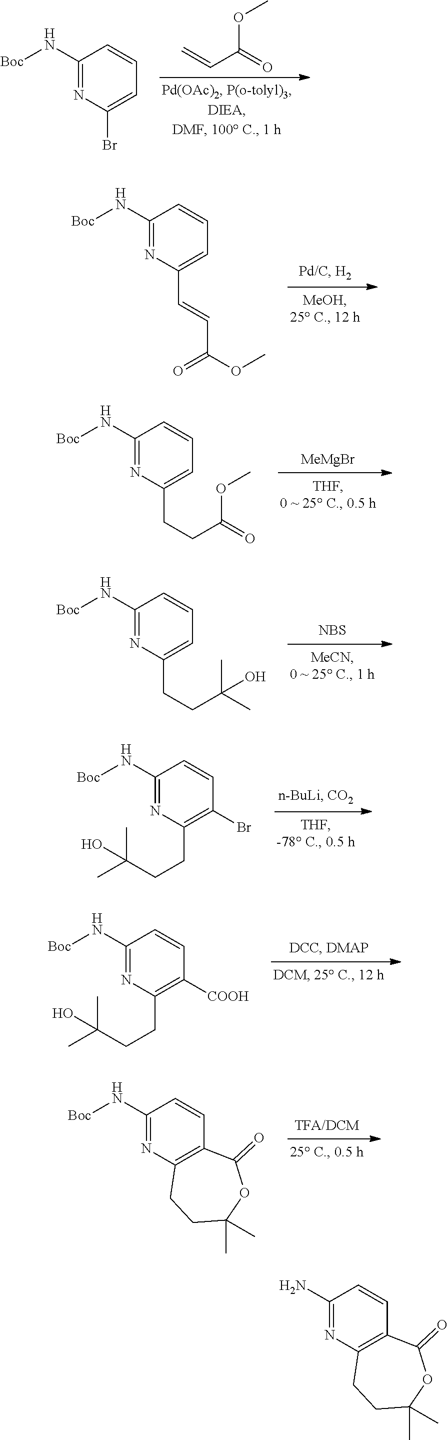

### Step 1: Methyl (E)-3-(6-((tert-butoxycarbonyl)amino)pyridin-2-yl)acrylate

Pd(OAc)₂ (1.23 g, 5.49 mmol), P(o-tolyl)₃ (2.51 g, 8.24 mmol) and diisopropylethylamine (71.0 g, 549 mmol, 95.7 mL) were added to a solution of tert-butyl (6-bromopyridin-2-yl)carbamate (15.0 g, 54.9 mmol) and methyl acrylate (18.9 g, 220 mmol, 19.8 mL) in N,N-dimethyl formamide (150 mL). The reaction mixture was stirred at 100° C. for 1 h, then was diluted EA (200 mL) and washed with brine (200 mL×3). The organic layers were dried over sodium sulfate, filtered and concentrated to give a residue. The residue was purified by flash-column chromatography on silica gel (gradient elution, 10% to 33% EA-petroleum ether) to give the title compound (7.00 g, 39% yield) as a yellow solid.

### Step 2: Methyl 3-(6-((tert-butoxycarbonyl)amino)pyridin-2-yl)propanoate

Pd/C (100 mg, 10% purity) was added to a solution of methyl (E)-3-(6-((tert-butoxy carbonyl)amino)pyridin-2-yl)acrylate (7.00 g, 25.2 mmol) in methanol (100 mL). The mixture was stirred at 25° C. for 12 h under hydrogen, then was filtered and concentrated to give a residue. The residue was purified by flash-column chromatography on silica gel (gradient elution, 10% to 33% EA-petroleum ether) to give the title compound (6.00 g, 18.6 mmol, 74% yield) as a yellow solid.

Step 3: tert-Butyl (6-(3-hydroxy-3-methylbutyl)pyridin-2-yl)carbamate Methyl magnesium bromide (3 M, 35.7 mL) was added to a solution of methyl 3-(6-((tert-butoxycarbonyl)amino)pyridin-2-yl)propanoate (6.00 g, 21.4 mmol) in tetrahydrofuran (100 mL) at 0° C. The reaction mixture was stirred at 25° C. for 0.5 h, then was poured into water (200 mL) and extracted with EA (50 mL×3). The combined organic layers were dried over sodium sulfate, filtered, and concentrated to give a residue. The residue was purified by flash-column chromatography on silica gel (gradient elution, 15% to 50% EA-petroleum ether) to give the title compound (5.00 g, 15.9 mmol, 74% yield) as a yellow solid.

### Step 4: tert-Butyl (5-bromo-6-(3-hydroxy-3-methylbutyl)pyridin-2-yl)carbamate

N-Bromosuccinimide (3.17 g, 17.8 mmol) in acetonitrile (50 mL) was added to a solution of tert-butyl (6-(3-hydroxy-3-methylbutyl)pyridin-2-yl)carbamate (5.00 g, 17.8 mmol) in acetonitrile (50 mL) at 0° C. The reaction mixture was stirred at 25° C. for 1 h, then was poured into water (200 mL) and extracted with EA (50 mL×3). The combined organic layers were dried over sodium sulfate, filtered, and concentrated to give a residue. The residue was purified by flash-column chromatography on silica gel (gradient elution, 10% to 33% EA-petroleum ether) to give the title compound (4.00 g, 62% yield) as a yellow solid. ¹H-NMR (400 MHz, CDCl₃): δ ppm 7.72 (d, J=8.4 Hz, 1H), 7.65 (d, J=8.8 Hz, 1H), 7.39 (S, 1H), 2.99-2.93 (m, 2H), 1.87-1.78 (m, 2H), 1.49 (s, 9H), 1.27 (s, 6H).

### Step 5: 6-((tert-Butoxycarbonyl)amino)-2-(3-hydroxy-3-methylbutyl)nicotinic acid

n-Butyllithium (2.5 M, 11.1 mL) was added to a solution of tert-butyl (5-bromo-6-(3-hydroxy-3-methylbutyl)pyridin-2-yl)carbamate (2.00 g, 5.57 mmol) in tetrahydrofuran (50 mL) at −78° C. The reaction mixture was stirred at −78° C. for 10 min, then carbon dioxide was added, and the mixture was stirred at −78° C. for 20 min. The reaction mixture was then poured into water (100 mL) and extracted with ethyl acetate (30 mL×3). The organic layers were discarded, and aqueous ammonium chloride solution was added to the aqueous layer to adjust pH<7. The mixture was extracted with EA (30 mL×5), and the combined organic layers was dried over sodium sulfate, filtered and concentrated to give the title compound (200 mg, crude) as a yellow solid.

### Step 6: tert-Butyl (7,7-dimethyl-5-oxo-5,7,8,9-tetrahydrooxepino[4,3-b]pyridin-2-yl)carbamate

Dicyclohexylcarbodiimide (229 mg, 1.11 mmol, 225 μL) was added to a solution of 6-((tert-butoxycarbonyl)amino)-2-(3-hydroxy-3-methylbutyl)nicotinic acid (180 mg, crude) and 4-N,N-dimethylaminopyridine (33.9 mg, 277 μmol) in dichloromethane (20 mL). The reaction mixture was stirred at 25° C. for 12 h, then was concentrated to give the residue. The residue purified by prep-TLC on silica gel (33% EA-petroleum ether) to give the title compound (30.0 mg, crude) as a yellow solid.

### Step 7: 2-Amino-7,7-dimethyl-8,9-dihydrooxepino[4,3-b]pyridin-5(7H)-one

The title compound was prepared from tert-butyl (7,7-dimethyl-5-oxo-5,7,8,9-tetrahydrooxepino[4,3-b]pyridin-2-yl)carbamate using a similar procedure as describe in Step 7 of Intermediate 8. ¹H-NMR (400 MHz, CDCl₃): δ ppm 7.92 (d, J=8.8 Hz, 1H), 6.36 (d, J=8.8 Hz, 1H), 5.17 (s, 2H), 2.96-2.92 (m, 2H), 2.11-2.09 (m, 2H), 1.33 (s, 6H).

### Example 1i

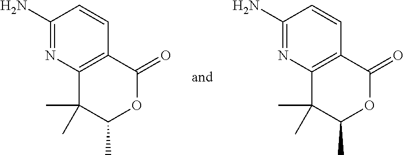

### Intermediates 12 and 13: (R)-2-Amino-7,8,8-trimethyl-7,8-dihydro-5H-pyrano[4,3-b]pyridin-5-one or (S)-2-amino-7,8,8-trimethyl-7,8-dihydro-5H-pyrano[4,3-b]pyridin-5-one

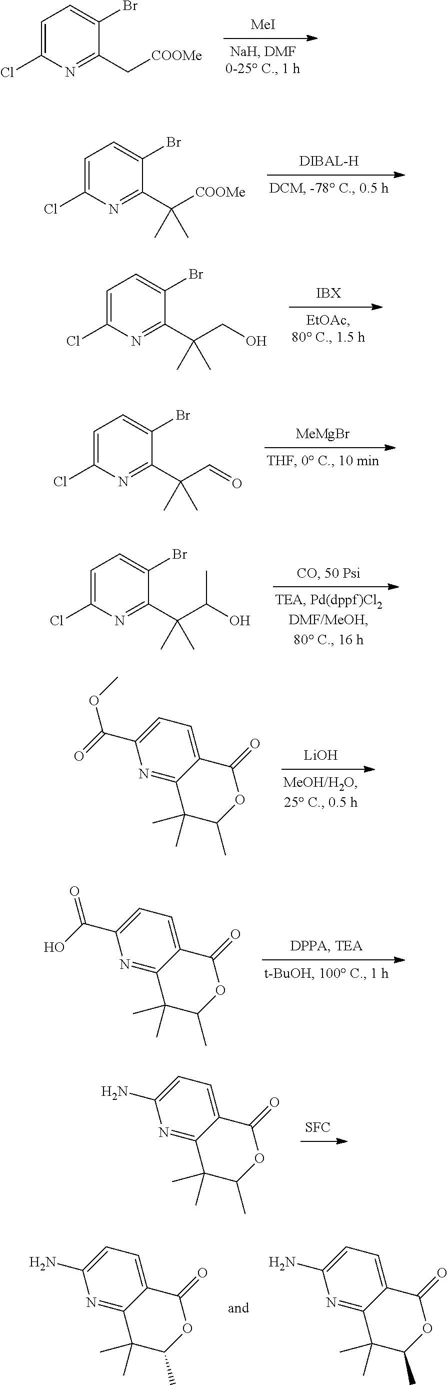

### Step 1: Methyl 2-(3-bromo-6-chloropyridin-2-yl)-2-methylpropanoate

Sodium hydride (2.91 g, 72.8 mmol, 60% purity) was added to a solution of methyl 2-(3-bromo-6-chloropyridin-2-yl)acetate (5.50 g, 20.8 mmol) in tetrahydrofuran (20 mL) at 0° C. The reaction mixture was stirred for 15 minutes at 0° C., then iodomethane (7.38 g, 51.9 mmol) was added. The reaction mixture was warmed to 25° C. and stirred for 45 minutes, then was quenched with water (30 mL) and extracted with EA (30 mL×2). The combined organic layers were concentrated to give a residue. The residue was purified by flash-column chromatography on silica gel (gradient elution, 0% to 10% EA-petroleum ether) to give the title compound (5.5 g, 90% yield) as a yellow oil. ¹H NMR (400 MHz, CD₃OD): δ ppm 7.97 (d, J=8.4 Hz, 1H), 7.20 (d, J=8.4 Hz, 1H), 3.95 (s, 2H), 1.50 (s, 6H).

### Step 2: 2-(3-Bromo-6-chloropyridin-2-yl)-2-methylpropan-1-ol

The title compound was prepared from methyl 2-(3-bromo-6-chloropyridin-2-yl)-2-methylpropanoate using a procedure similar to that described in Step 3 of Intermediate 7.

### Steps 3-7: (rac)-2-Amino-7,8,8-trimethyl-7,8-dihydro-5H-pyrano[4,3-b]pyridin-5-on

The title compound was prepared from 2-(3-bromo-6-chloropyridin-2-yl)-2-methylpropan-1-ol using a similar procedure as described in Steps 1-5 of Intermediate 8. C₁₁H₁₄N₂O₂ requires: 206, found: 207 [M+H]⁺.

### Step 8: (R)-2-Amino-7,8,8-trimethyl-7,8-dihydro-5H-pyrano[4,3-b]pyridin-5-one or (S)-2-amino-7,8,8-trimethyl-7,8-dihydro-5H-pyrano[4,3-b]pyridin-5-one

(rac)-2-Amino-7,8,8-trimethyl-7,8-dihydro-5H-pyrano[4,3-b]pyridin-5-one (120 mg) was separated by SFC (column: REGIS (s,s) WHELK-01 (250 mm×50 mm, 10 um), MeOH gradient in CO₂ with 0.1% NH₄OH) to give two peaks separately. The first eluting isomer (Intermediate 12, 60 mg, 50% yield) and second eluting isomer (Intermediate 13, 60 mg, 50% yield) were obtained as yellow solids.

### Example 1j

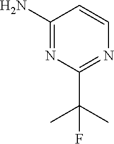

### Intermediate 14: 2-(2-Fluoropropan-2-yl)pyrimidin-4-amine

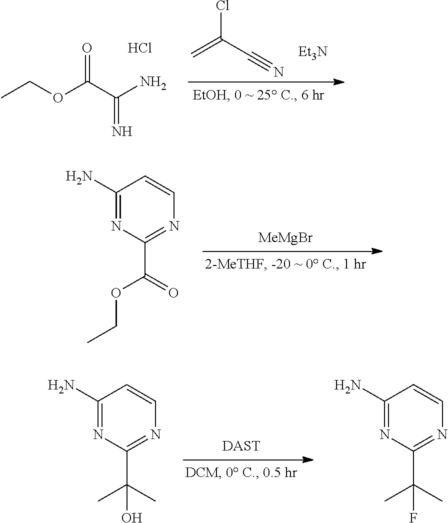

### Step 1: Ethyl 4-aminopyrimidine-2-carboxylate

Triethylamine (285 g, 2.81 mol, 2.00 equiv) was added dropwise to a solution of ethyl 2-amino-2-iminoacetate hydrochloride (215 g, 1.41 mol, 1.00 equiv) and 2-chloroprop-2-enenitrile (112 mL, 1.41 mol, 1.00 eq) in EtOH (1.8 L) at 0° C. The mixture was allowed to warm to 25° C. and stirred at 25° C. for 6 h. The reaction mixture was concentrated, and the residue was partitioned between water (10.0 L) and EA (5.0 L). The layers were separated, and the aqueous layer was extracted with EA (5.0 L×2). The organic layers were combined and dried over Na₂SO₄. The dried solution was filtered, and the filtrate was concentrated to give the title compound (270 g, crude) as a dark brown solid.

### Step 2: 2-(4-Aminopyrimidin-2-yl)propan-2-ol

Methylmagnesium bromide (900 mL, 3.0 M, 5.0 eq) was added to a solution of ethyl 4-aminopyrimidine-2-carboxylate (90.0 g, 538 mmol, 1.00 eq) in 2-MeTHF (1.00 L) at −20° C. The reaction mixture was allowed warm to 0° C. and stirred at that temperature for 1 h. The reaction mixture was diluted with saturated aqueous ammonium chloride solution (10.0 L) and the aqueous layer was extracted with EA (3.0 L×4). The organic layers were combined and dried over Na₂SO₄, and the dried solution was filtered. The filtrate was concentrated to give the title compound (50 g, crude) as a dark brown solid.

### Step 3: 2-(2-Fluoropropan-2-yl)pyrimidin-4-amine

DAST (414 mL, 3.13 mol, 10.0 eq) was added to a solution of 2-(4-aminopyrimidin-2-yl)propan-2-ol (48.0 g, 313 mmol, 1.00 eq) in DCM (1.30 L) at 0° C. The reaction mixture was stirred at 0° C. for 0.5 h, and then was diluted with water (4.50 L). The reaction mixture was adjusted to pH=7-8 with aqueous sodium hydroxide solution (0.50 L) and sodium carbonate solution (200 mL), then was extracted with EA (2.00 L×3). The organic layers were combined and dried over Na₂SO₄, and the dried solution was filtered and concentrated. The residue was purified by rp-HPLC (neutral condition) to give the title compound (13.0 g) as a white solid. ¹H NMR (400 MHz DMSO-d6) δ 8.05-8.07 (d, J=5.6 Hz), 6.93 (s, 1H), 6.32-6.33 (d, J=5.6 Hz, 1H), 1.63 (s, 3H), 1.58 (s, 3H).

### Example 1k

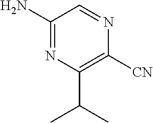

### Intermediate 15: 5-Amino-3-isopropylpyrazine-2-carbonitrile

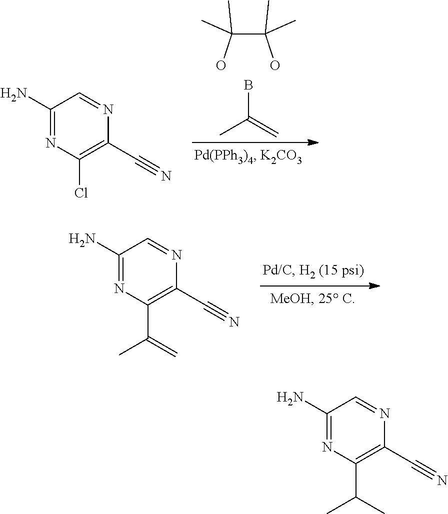

### Step 1: 5-Amino-3-(prop-1-en-2-yl)pyrazine-2-carbonitrile

A mixture of 5-amino-3-chloropyrazine-2-carbonitrile (50.0 g, 324 mmol, 1.00 eq), 4,4,5,5-tetramethyl-2-(prop-1-en-2-yl)-1,3,2-dioxaborolane (81.54 g, 485.3 mmol, 1.50 eq), K₂CO₃ (89.42 g, 647.0 mmol, 2.00 eq), and Pd(PPh₃)₄(18.69 g, 16.18 mmol, 0.05 eq) in dioxane (250 mL) and H₂O (50 mL) was stirred at 100° C. for 20 h under N₂. The reaction mixture was then cooled to ambient temperature and diluted with EA (800 mL) and H₂O (300 mL). The biphasic mixture was then filtered through celite, then partitioned. The organic layer was washed with brine (500 mL×4), and then the organic layer was dried over Na₂SO₄, filtered and concentrated under vacuum. The residue was purified by flash-column chromatography on silica gel (gradient elution, 10% to 33% EA-petroleum ether) to give the title compound (41.0 g, 39.0% yield) as a light yellow solid.

### Step 2: 5-Amino-3-isopropylpyrazine-2-carbonitrile

Pd/C (10 wt %, 10.0 g) was added to a solution of 5-amino-3-(prop-1-en-2-yl)pyrazine-2-carbonitrile (45.0 g, 281 mmol, 1.00 eq) in MeOH (800 mL). The suspension was degassed under vacuum and purged with H₂ three times. The reaction mixture was stirred under H₂ (15 psi) at 25° C. for 16 h. The mixture was filtered through celite and the filtrate was concentrated under vacuum. The residue was treated with petroleum ether/EA (110 mL, 10:1) and stirred at 25° C. for 10 min, then filtered. The filter cake was dried under vacuum to give the title compound (39.5 g, 85.6% yield) as a light yellow solid. MS (ES+) C₈H₁₀N₄ requires: 162, found: 163[M+H]⁺. ¹H NMR: 400 MHz CDCl₃ δ: 7.83 (s, 1H), 5.07 (br s, 2H), 3.43-3.33 (m, 1H), 1.28 (s, 3H), 1.26 (s, 3H).

### Example 1l

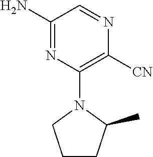

### Intermediate 16: (S)-5-Amino-3-(2-methylpyrrolidin-1-yl)pyrazine-2-carbonitrile

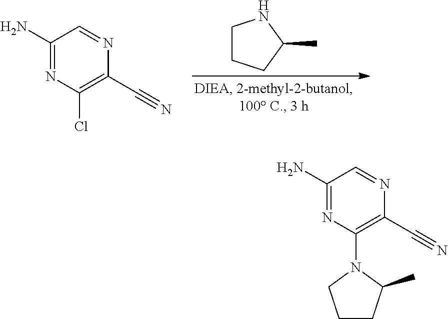

### Step 1: (S)-5-Amino-3-(2-methylpyrrolidin-1-yl)pyrazine-2-carbonitrile

N,N-Diisopropylethylamine (0.237 mL, 1.36 mmol, 3.00 equiv) was added to a mixture of 5-amino-3-chloropyrazine-2-carbonitrile (70.0 mg, 453 umol, 1.00 equiv) and (S)-2-methylpyrrolidine (HCl salt, 71.6 mg, 589 umol, 1.30 equiv) in 2-methyl-2-butanol (2 mL). The reaction mixture was stirred at 100° C. for 3 h, then was poured into 20 mL of water and extracted with EA (15 mL×3). The organic layers were dried by anhydrous sodium sulfate, filtered and concentrated under reduced pressure to give the title compound (90.0 mg, crude) as a yellow solid that was used without further purification. MS (ES+) C₁₀H₁₃N₅ requires: 203, found: 204[M+H]⁺.

### Example 1m

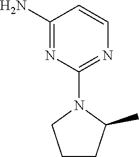

### Intermediate 17: (S)-2-(2-Methylpyrrolidin-1-yl)pyrimidin-4-amine

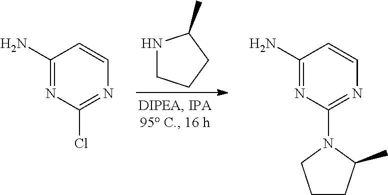

### Step 1: (S)-2-(2-Methylpyrrolidin-1-yl)pyrimidin-4-amine

A mixture of 2-chloropyrimidin-4-amine (1.0 g, 7.72 mmol), (2S)-2-methylpyrrolidine hydrochloride (1.03 g, 8.49 mmol), and DIPEA (2.02 mL, 11.6 mmol) in IPA (15 mL) was heated to 95° C. in a sealed vial. The reaction mixture was stirred at 95° C. for 16 h, then was concentrated to give a residue. The residue was purified by flash-column chromatography on silica gel (0 to 10% MeOH-DCM) to give the title compound as a white solid. MS (ES+) C₉H₁₄N₄ requires: 178, found: 179[M+H]⁺.

### Example 1n

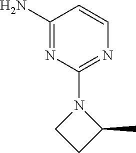

### Intermediate 18: (S)-2-(2-Methylazetidin-1-yl)pyrimidin-4-amine

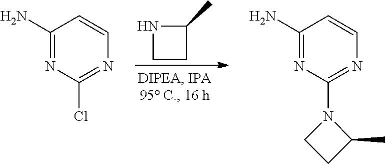

### Step 1: (S)-2-(2-Methylazetidin-1-yl)pyrimidin-4-amine

The title compound was prepared from 2-chloropyrimidin-4-amine and (S)-2-methylazetidine hydrochloride using a similar procedure as described above for Intermediate 18. MS (ES+) C₈H₁₂N₄ requires: 164, found: 165[M+H]⁺.

### Example 2: Synthesis of arylchloride Intermediates

### Example 2a

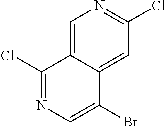 [SMILES: Clc1cc2c(Br)cnc(Cl)c2cn1]

Intermediate 19: 4-Bromo-1,6-dichloro-2,7-naphthyridine

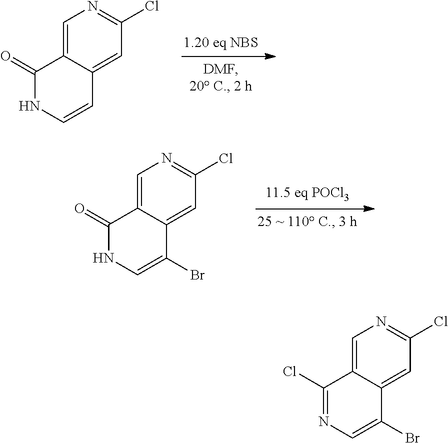 [SMILES: O=c1[nH]ccc2cc(Cl)ncc12 ; O=c1[nH]cc(Br)c2cc(Cl)ncc12 ; Clc1cc2c(Br)cnc(Cl)c2cn1]

### Step 1: 4-Bromo-6-chloro-2,7-naphthyridin-1(2H)-one

NBS (70.9 g, 398 mmol, 1.20 eq) was added to a solution of 6-chloro-2,7-naphthyridin-1(2H)-one (60.0 g, 332 mmol, 1.00 eq) in DMF (600 mL). The reaction mixture was stirred at 20° C. for 2 h, then was poured into water (1 L) and filtered. The filter cake was dried under vacuum to give 4-bromo-6-chloro-2,7-naphthyridin-1(2H)-one (90.8 g, crude) as a brown solid. MS (ES+) C₈H₄BrClN₂O requires: 260, found: 261[M+H]⁺.

### Step 2: 4-Bromo-1,6-dichloro-2,7-naphthyridine

4-Bromo-6-chloro-2,7-naphthyridin-1(2H)-one (70.8 g, 272 mmol, 1.00 eq) was added in portions to POCl₃ (484 g, 3.16 mol, 293 mL, 11.5 eq) at 25° C. The reaction mixture was then stirred at 110° C. for 3 h. The reaction mixture was then concentrated under vacuum, and the residue was adjusted to pH=8 with saturated aqueous Na₂CO₃ at 25° C. The mixture was extracted with DCM (500 mL×3), washed with brine (500 mL), dried over Na₂SO₄, filtered and concentrated under vacuum to give the title compound (75.0 g, 269 mmol, 98.9% yield) as a yellow solid. MS (ES+) C₈H₃BrCl₂N₂ requires: 278, found: 279[M+H]⁺.

### Example 2b

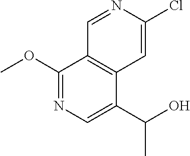 [SMILES: COc1ncc(C(C)O)c2cc(Cl)ncc12]

### Intermediate 20: 1-(6-Chloro-1-methoxy-2,7-naphthyridin-4-yl)ethan-1-ol

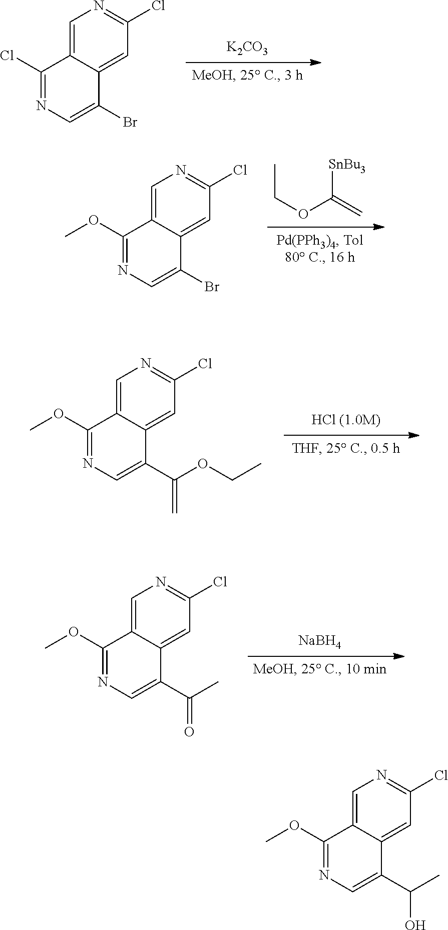

### Step 1: 4-Bromo-6-chloro-1-methoxy-2,7-naphthyridine

A suspension of 4-bromo-1,6-dichloro-2,7-naphthyridine (75.0 g, 269 mmol, 1.00 eq), K₂CO₃ (111 g, 809 mmol, 3.00 eq) in MeOH (3 L) was stirred at 25° C. for 16 h. The reaction mixture was then concentrated under vacuum, and the residue was dissolved in H₂O (300 mL) and extracted with DCM (100 mL×2). The combined organic layers were concentrated under vacuum to give a residue. The residue was triturated in PE/EA (40 mL 20:1) and filtered. The filter cake was dried under vacuum to give the title compound (47.0 g, 171 mmol, 63.6% yield) as a yellow solid.

### Step 2: 6-Chloro-4-(1-ethoxyvinyl)-1-methoxy-2,7-naphthyridine

A solution of 4-bromo-6-chloro-1-methoxy-2,7-naphthyridine (47.0 g, 171 mmol, 1.00 eq), tributyl(1-ethoxyvinyl)stannane (74.4 g, 206 mmol, 69.6 mL, 1.20 eq) and Pd(PPh₃)₄(19.8 g, 17.1 mmol, 0.10 eq) in toluene (500 mL) was stirred at 80° C. for 16 h under N₂. The reaction mixture was then cooled to 20° C. and poured into saturated aqueous KF solution (500 mL) and stirred for 1 h. The aqueous mixture was extracted with EA (300 mL×3), and the organic layers were combined. The combined organic layer was concentrated under vacuum to give the title compound (64.0 g, crude) as a yellow oil. MS (ES+) C₁₃H₁₃ClN₂O₂ requires: 264, found: 265[M+H]⁺.

### Step 3: 1-(6-Chloro-1-methoxy-2,7-naphthyridin-4-yl)ethan-1-one

Aqueous HCl (1.50 M, 20.1 mL, 0.10 eq) was added to a solution of 6-chloro-4-(1-ethoxyvinyl)-1-methoxy-2,7-naphthyridine (80.0 g, 302 mmol, 1.00 eq) in THF (480 mL) and H₂O (80 mL). The reaction mixture was stirred at 25° C. for 1 h. The reaction mixture was then poured into saturated aqueous NaHCO₃ solution (500 mL) and extracted with EA (300 mL×2). The organic layers were combined and concentrated under vacuum. The residue was purified by flash-column chromatography on silica gel (gradient elution, 5% to 50% EA-PE) to give the title compound (28.0 g, 118 mmol, 39.1% yield) as a white solid. MS (ES+) C₁₁H₉C₁N₂O₂ requires: 236, found: 237[M+H]⁺.

### Step 4: 1-(6-Chloro-1-methoxy-2,7-naphthyridin-4-yl)ethan-1-ol

To a solution of 1-(6-chloro-1-methoxy-2,7-naphthyridin-4-yl)ethan-1-one (500 mg, 2.11 mmol) in MeOH (10 mL) was added NaBH₄ (120 mg, 3.17 mmol). The mixture was stirred at 25° C. for 10 min, then was quenched by addition of water (5 mL) and extracted with EA (5 mL×3). The combined organic layers were concentrated to give a residue. The residue was purified by column chromatography (SiO₂, PE/EA=10/1 to 1:1) to give the title compound (400 mg, 79% yield) as an off-white solid.

### Example 2c

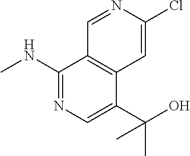 [SMILES: CNc1ncc(C(C)(C)O)c2cc(Cl)ncc12]

### Intermediate 21: 2-(6-Chloro-1-(methylamino)-2,7-naphthyridin-4-yl)propan-2-ol

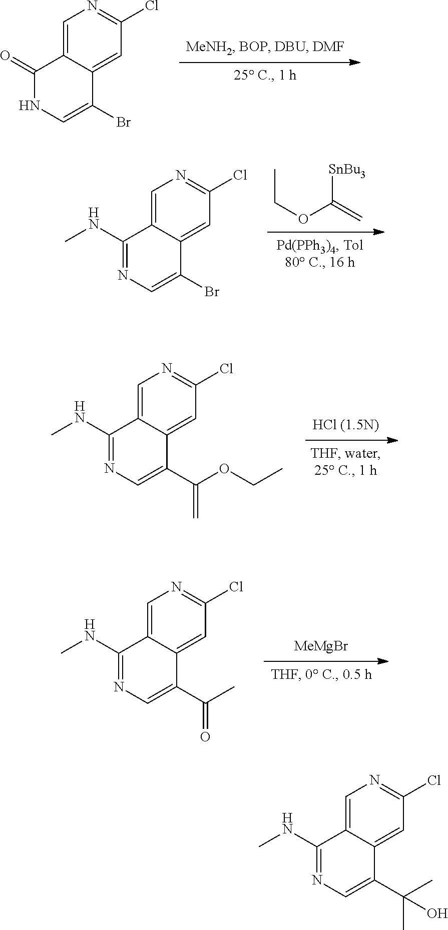

### Step 1: 4-Bromo-6-chloro-N-methyl-2,7-naphthyridin-1-amine

To a solution of 4-bromo-6-chloro-2,7-naphthyridin-1(2H)-one (600 mg, 2.31 mmol) in DMF (15 mL) was added DBU (528 mg, 3.47 mmol, 523 uL) and BOP (1.53 g, 3.47 mmol), then methylamine (2 M, 3.04 g) was added to the reaction mixture and it was stirred at 25° C. for 1 h. The reaction mixture was then diluted with water (10 mL), filtered and the filter cake was dried to give the title compound (440 mg, 70% yield) as a yellow solid that was used without further purification. MS (ES+) C₉H₇BrClN₃ requires: 271, found: 272[M+H]⁺.

### Steps 2-3: 1-(6-Chloro-1-(methylamino)-2,7-naphthyridin-4-yl)ethan-1-one

The title compound was prepared from 4-Bromo-6-chloro-N-methyl-2,7-naphthyridin-1-amine using a procedure similar to the that described in Steps 2-3 of Intermediate 20.

Step 4: 2-(6-Chloro-1-(methylamino)-2,7-naphthyridin-4-yl)propan-2-ol

To a solution of 1-(6-chloro-1-(methylamino)-2,7-naphthyridin-4-yl)ethan-1-one (100 mg, 424 umol) in THF (8 mL) was added MeMgBr (3 M, 424 uL) at 0° C. The reaction mixture was stirred at 0° C. for 0.5 h, then was quenched by addition of water (17 mL) and extracted with EA (15 mL×3). The combined organic layers were dried over anhydrous sodium sulfate, filtered and concentrated to give a residue. The residue was purified by prep-TLC (silica, PE:EA=1:1) to give the title compound (50.0 mg, 47% yield) as a yellow solid. MS (ES+) C₁₂H₁₄ClN₃O requires: 251, found: 252[M+H]⁺.

### Example 2d

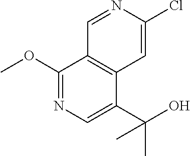 [SMILES: COc1ncc(C(C)(C)O)c2cc(Cl)ncc12]

### Intermediate 22: 2-(6-Chloro-1-methoxy-2,7-naphthyridin-4-yl)propan-2-ol

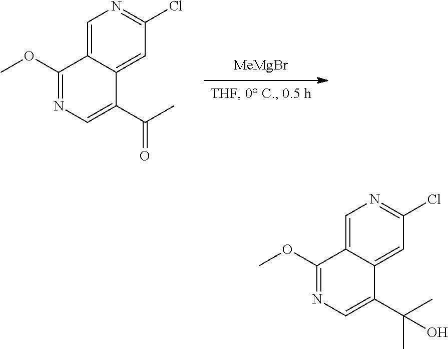 [SMILES: "MeMgBr" ; COc1ncc(C(C)=O)c2cc(Cl)ncc12 ; "THF, 0° C., 0.5 h" ; COc1ncc(C(C)(C)O)c2cc(Cl)ncc12]

### Step 1: 2-(6-Chloro-1-methoxy-2,7-naphthyridin-4-yl)propan-2-ol

To a solution of 1-(6-chloro-1-methoxy-2,7-naphthyridin-4-yl)ethan-1-one (220 mg, 930 umol) in THF (10 mL) was added MeMgBr (3 M, 930 uL) at 0° C. The reaction mixture was stirred at 0° C. for 0.5 h, then was quenched by addition of water (17 mL) extracted with EA (15 mL×3). The combined organic layers were dried over anhydrous sodium sulfate, filtered and concentrated to give the title compound (200 mg, 85% yield) a yellow solid that was used in the next step without further purification. MS (ES+) C₁₂H₁₃ClN₂O₂ requires: 252, found: 253[M+H]⁺.

### Example 2e

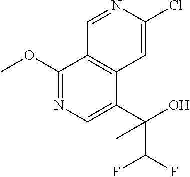 [SMILES: COc1ncc(C(C)(O)C(F)F)c2cc(Cl)ncc12]

### Intermediate 23: 2-(6-Chloro-1-methoxy-2,7-naphthyridin-4-yl)-1,1-difluoropropan-2-ol

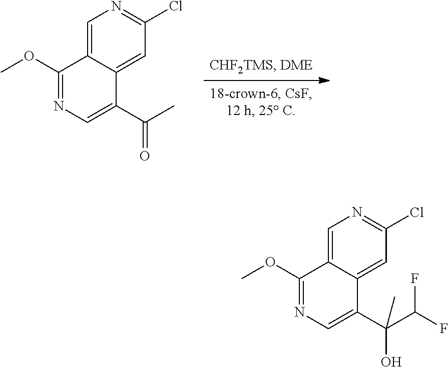 [SMILES: COc1ncc(C(C)=O)c2cc(Cl)ncc12 ; "18-crown-6, CsF,12 h, 25° C." ; "CHF2TMS, DME" ; COc1ncc(C(C)(O)C(F)F)c2cc(Cl)ncc12]

### Step 1: 2-(6-Chloro-1-methoxy-2,7-naphthyridin-4-yl)-1,1-difluoropropan-2-ol

To a solution of 1-(6-chloro-1-methoxy-2,7-naphthyridin-4-yl)ethan-1-one (100 mg, 423 umol) and difluoromethyl(trimethyl)silane (105 mg, 845 umol) in DME (3 mL) was added 18-crown-6 (78.2 mg, 296 umol) and CsF (44.9 mg, 296 umol). The reaction mixture was stirred at 25° C. for 12 h, then was filtered and concentrated to give a residue. The residue was purified by prep-TLC (PE:EA=5:1) to give the title compound (70.0 mg, 57% yield) as a yellow solid. MS (ES+) C₁₂H₁₁ClF₂N₂O₂ requires: 288, found: 289[M+H]⁺.

### Example 2f

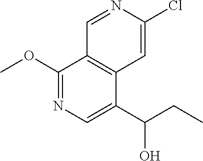 [SMILES: CCC(O)c1cnc(OC)c2cnc(Cl)cc12]

### Intermediate 24: 1-(6-Chloro-1-methoxy-2,7-naphthyridin-4-yl)propan-1-ol

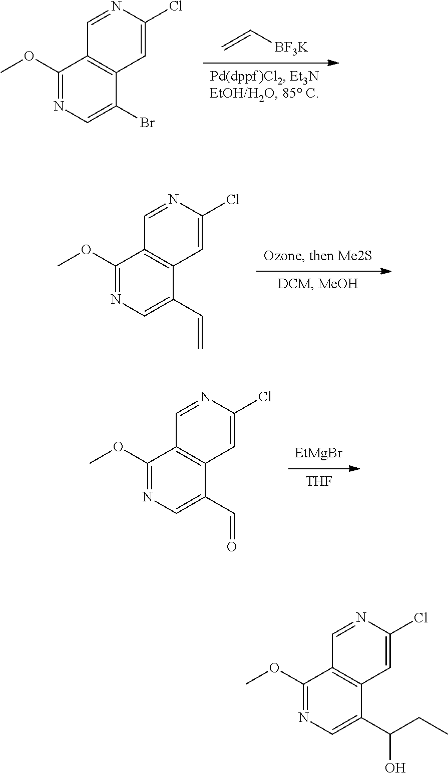

### Step 1: 6-Chloro-1-methoxy-4-vinyl-2,7-naphthyridine

A mixture of 4-bromo-6-chloro-1-methoxy-2,7-naphthyridine (23.5 g, 85.9 mmol), Potassium trifluoro(vinyl)borate (15.0 g, 112 mmol), Et₃N (17.4 g, 172 mmol) and Pd(dppf)Cl₂ (6.29 g, 8.59 mmol) in H₂O (48 mL) and EtOH (480 mL) was stirred at 80° C. for 12 h. The reaction mixture was then diluted with EA (300 mL) and brine (100 mL). The organic layer was dried over Na₂SO₄, filtered and concentrated. The residue was purified by column chromatography on silica gel (PE/EA=10/1) to give the title compound (10 g, crude) as a white solid that was used without further purification.

### Step 2: 6-Chloro-1-methoxy-2,7-naphthyridine-4-carbaldehyde

A solution of 6-chloro-1-methoxy-4-vinyl-2,7-naphthyridine (800 mg, 3.63 mmol) in DCM (6 mL) and MeOH (1 mL) was cooled to −78° C., then ozone was bubbled into the reaction mixture. After 10 min, the reaction mixture was flushed with nitrogen, and then Me₂S (4.45 g, 71.6) was added and the mixture was allowed to warm to 25° C. After stirring for 50 min at 25° C., the reaction mixture was poured into water (20 mL) and extracted with EA (20 mL×3). The combined organic layers were washed with water (20 mL×3), dried over anhydrous sodium sulfate, filtered, and concentrated to give a residue. The residue was purified by flash chromatography on silica gel (PE/EA=10:1-1:1), to give the title compound (400 mg, 50% yield) as a yellow solid.

### Step 3: 1-(6-Chloro-1-methoxy-2,7-naphthyridin-4-yl)propan-1-ol

To a solution of 6-chloro-1-methoxy-2,7-naphthyridine-4-carbaldehyde (100 mg, 284 umol) in THF (2 mL) was added EtMgBr (5.65 g, 39.8 mmol) in one portion at 20° C. The reaction mixture was stirred at 20° C. for 1 h, then was quenched with water (20 mL) and extracted with EA (20 mL×3). The combined organic layers were washed with water (20 mL×3), dried over sodium sulfate, filtered and concentrated to give the title compound (50 mg, 48% yield) as a yellow oil. ¹H NMR (400 MHz, CDCl₃) δ ppm 9.41 (s, 1H), 8.20 (s, 1H), 8.03 (s, 1H), 5.00-4.96 (m, 1H), 4.17 (s, 3H), 2.01-1.94 (m, 2H), 1.00 (t, J=7.6 Hz, 3H).

### Example 2g

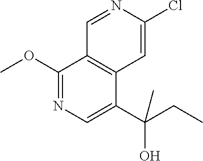 [SMILES: CCC(C)(O)c1cnc(OC)c2cnc(Cl)cc12]

### Intermediate 25: 2-(6-Chloro-1-methoxy-2,7-naphthyridin-4-yl)butan-2-ol

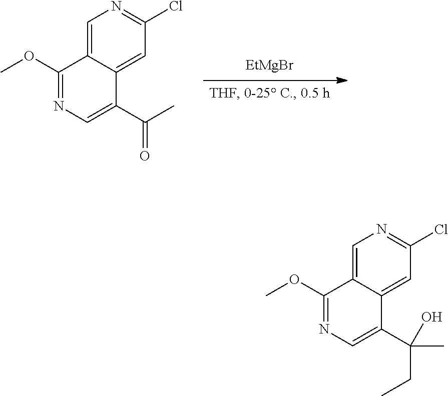 [SMILES: "EtMgBr" ; COc1ncc(C(C)=O)c2cc(Cl)ncc12 ; "THF, 0-25° C., 0.5 h" ; CCC(C)(O)c1cnc(OC)c2cnc(Cl)cc12]

### Step 1: 2-(6-Chloro-1-methoxy-2,7-naphthyridin-4-yl)butan-2-ol

To a solution of 1-(6-chloro-1-methoxy-2,7-naphthyridin-4-yl)ethan-1-one (1 g, 4.23 mmol) in THF (20 mL) was added EtMgBr (3 M, 4.23 mL) at 0° C. The cooling bath was removed, and the reaction mixture was stirred at 25° C. for 0.5 h. The reaction mixture was then partitioned between EA (60 mL) and saturated aqueous ammonium chloride solution (40 mL). The organic layer was washed with saturated aqueous sodium chloride solution (40 mL×3), dried over anhydrous sodium sulfate, filtered and concentrated to give a residue. The residue was purified by column chromatography (SiO₂, PE/EA=100/1 to 2/1) to give the title compound (400 mg, 28% yield) as a white solid. MS (ES+) C₁₃H₁₅ClN₂O₂ requires: 266, found: 267[M+H]⁺. ¹H-NMR (400 MHz, CD₃OD): δ ppm 9.36 (s, 1H), 8.67 (s, 1H), 8.20 (s, 1H), 4.16 (s, 3H), 2.08-2.04 (m, 2H), 1.70 (s, 3H), 0.81 (d, J=7.6 Hz, 3H).

### Example 2h

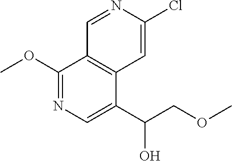

### Intermediate 26: 1-(6-Chloro-1-methoxy-2,7-naphthyridin-4-yl)-2-methoxyethan-1-ol

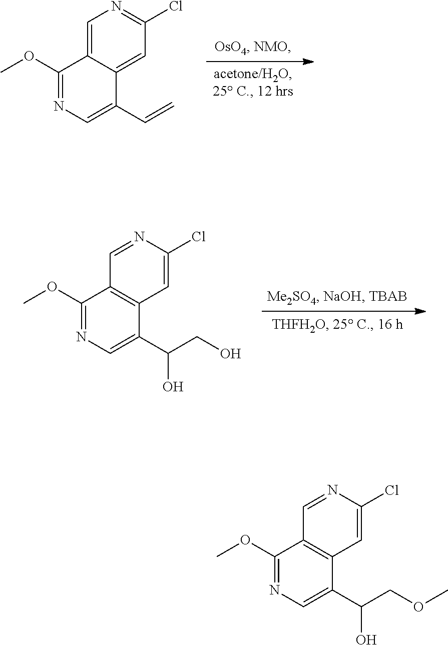

### Step 1: 1-(6-Chloro-1-methoxy-2,7-naphthyridin-4-yl)ethane-1,2-diol

To a mixture of 6-chloro-1-methoxy-4-vinyl-2,7-naphthyridine (15 g, 68.0 mmol) and NMO (15.9 g, 136 mmol, 14.4 mL) in acetone (300 mL) and H₂O (75 mL) was added OsO₄ (173 mg, 680 umol) in one portion. The reaction mixture was stirred at 25° C. for 12 h, then was quenched with saturated aqueous Na₂SO₃ aqueous solution (100 mL). The mixture was filtered, and the filtrate was concentrated to remove the organic solvents. The water layer was extracted with EA (3×200 mL). The combined organic layers were dried over Na₂SO₄, filtered and concentrated to give the title compound (9.8 g, crude) as a yellow solid that was used in the next step without further purification.

### Step 2: 1-(6-Chloro-1-methoxy-2,7-naphthyridin-4-yl)-2-methoxyethan-1-ol

To a solution of 1-(6-chloro-1-methoxy-2,7-naphthyridin-4-yl)ethane-1,2-diol (1 g, 3.93 mmol) and TBAB (127 mg, 393 umol) in H₂O (4 mL) and THF (20 mL) was added a solution of NaOH (471 mg, 11.8 mmol) in H₂O (4 mL), then Me₂SO₄ (594 mg, 4.71 mmol) was added. The reaction mixture was stirred at 25° C. for 4 h, and then more Me₂SO₄ (594 mg, 4.71 mmol) was added. The reaction mixture was stirred at 25° C. for an additional 12 h, then the mixture was partitioned between EA (150 mL) and H₂O (50 mL). The organic layer was dried over Na₂SO₄, filtered and concentrated. The residue was purified by silica gel column (PE/EA=1:2), then purified by prep-HPLC (column: Waters Xbridge C18 150*50 mm*10 um; mobile phase: [water (0.05% ammonia hydroxide v/v)—ACN]; B %: 18%-48%, 11 min) to give the title compound as a 1:1 mixture with a regioisomer. The mixture was separated by chiral SFC (column: Daicel Chiralpak AD (250 mm*30 mm, 10 um); mobile phase: [0.1% NH₃H₂O ETOH]; B %: 25%-25%) to give the title compound as the second eluting peak. MS (ES+) C₁₂H₁₃ClN₂O₃ requires: 268, found: 269[M+H]⁺. ¹H NMR (400 MHz, CD₃Cl): δ ppm 9.43 (s, 1H), 8.19 (s, 1H), 8.00 (s, 1H), 4.72-4.65 (m, 1H), 4.18 (s, 3H), 3.95-3.85 (m, 1H), 3.76-3.70 (m, 1H), 3.37 (s, 3H), 2.36-2.30 (m, 1H).

### Example 2i

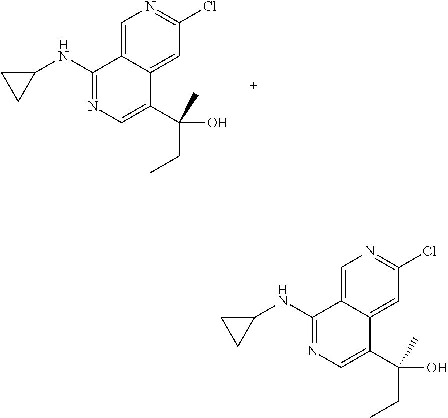 [SMILES: CC[C@@](C)(O)c1cnc(NC2CC2)c2cnc(Cl)cc12 ; CC[C@](C)(O)c1cnc(NC2CC2)c2cnc(Cl)cc12]

### Intermediates 27 and 28: (R)-2-(6-Chloro-1-(cyclopropylamino)-2,7-naphthyridin-4-yl)butan-2-ol and (S)-2-(6-chloro-1-(cyclopropylamino)-2,7-naphthyridin-4-yl)butan-2-ol

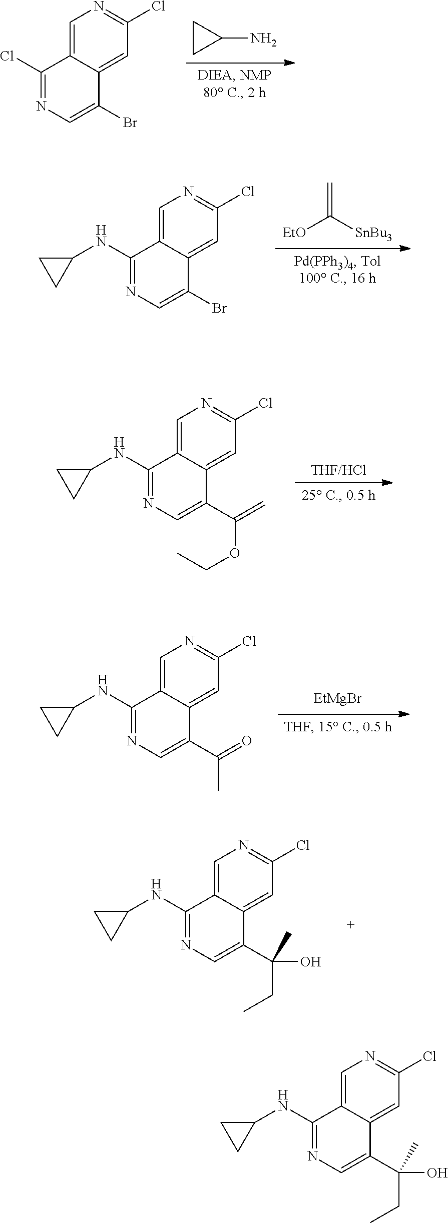

### Step 1: 4-Bromo-6-chloro-N-cyclopropyl-2,7-naphthyridin-1-amine

A solution of 4-bromo-1,6-dichloro-2,7-naphthyridine (2.00 g, 7.20 mmol), cyclopropanamine (493 mg, 8.64 mmol) and DIPEA (2.79 g, 21.6 mmol) in NMP (20 mL) was stirred at 80° C. for 2 h. The reaction mixture was then poured into water (200 mL) and filtered. The filter cake was dried to give the title compound (2.50 g, crude) as a yellow solid that was used in the next step without further purification. MS (ES+) C₁₁H₉BrClN₃ requires: 297, found: 298[M+H]⁺.

### Steps 2-3: 1-(6-Chloro-1-(cyclopropylamino)-2,7-naphthyridin-4-yl)ethan-1-one

The title compound was prepared from 4-bromo-6-chloro-N-cyclopropyl-2,7-naphthyridin-1-amine using a procedure similar to the that described in Steps 2-3 of Intermediate 20.

### Step 4: (R)-2-(6-Chloro-1-(cyclopropylamino)-2,7-naphthyridin-4-yl)butan-2-ol and (S)-2-(6-chloro-1-(cyclopropylamino)-2,7-naphthyridin-4-yl)butan-2-ol

To a solution of 1-(6-chloro-1-(cyclopropylamino)-2,7-naphthyridin-4-yl)ethan-1-one (850 mg, 3.25 mmol) in THF (170 mL) was added EtMgBr (3 M, 3 mL). The reaction mixture was stirred at 15° C. for 0.5 h, then was quenched by addition of saturated ammonium chloride solution (200 mL) and extracted with EA (200 mL×3). The combined organic layers were dried over anhydrous sodium sulfate, filtered and concentrated to give a residue. The residue was purified by silica gel chromatography (PE:EA=1:0 to 1:1) to give a racemic mixture of the title compounds (360 mg, 37% yield) a yellow solid. The racemate was separated by chiral SFC (column: Daicel Chiralpak IC (250 mm*30 mm, 10 um); mobile phase: [0.1% NH₃H₂O MEOH]; B %: 40%-40%) to give the first of (R)-2-(6-chloro-1-(cyclopropylamino)-2,7-naphthyridin-4-yl)butan-2-ol or (S)-2-(6-chloro-1-(cyclopropylamino)-2,7-naphthyridin-4-yl)butan-2-ol (peak 1, Intermediate 27, 120 mg, 33% yield) as a yellow solid and the second one of (R)-2-(6-chloro-1-(cyclopropylamino)-2,7-naphthyridin-4-yl)butan-2-ol or (S)-2-(6-chloro-1-(cyclopropylamino)-2,7-naphthyridin-4-yl)butan-2-ol (peak 2, Intermediate 28, 120 mg, 33%) as a yellow solid. MS (ES+) C₁₅H₁₈ClN₃O requires: 291, found: 292[M+H]⁺.

### Example 2j

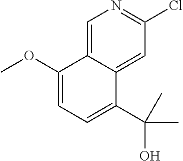 [SMILES: COc1ccc(C(C)(C)O)c2cc(Cl)ncc12]

### Intermediate 29: 2-(3-chloro-8-methoxyisoquinolin-5-yl)propan-2-ol

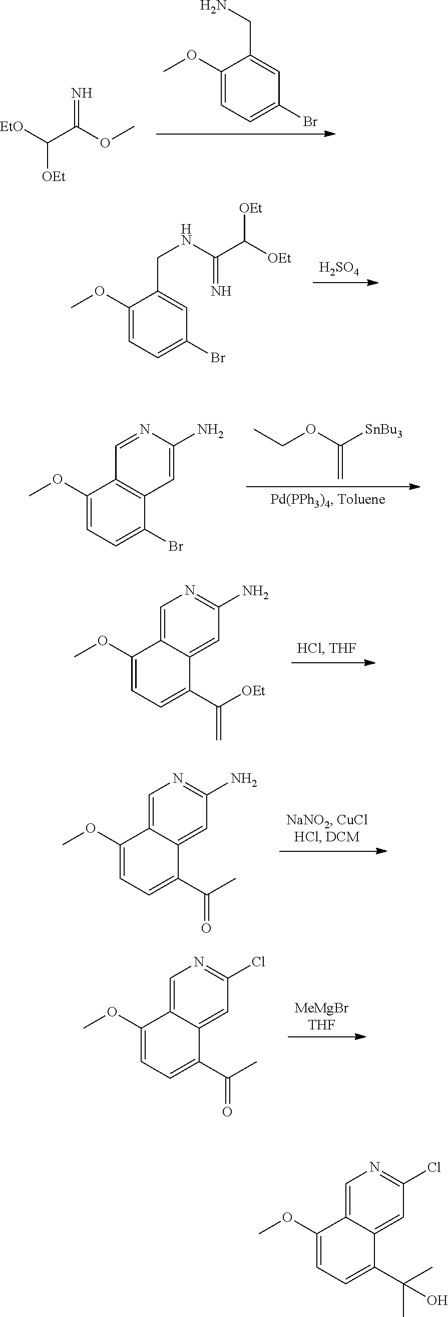

### Step 1: N-(5-Bromo-2-methoxybenzyl)-2,2-diethoxyacetimidamide

Methyl 2,2-diethoxyethanimidate (8.9 g, 55.5 mmol) was added to a solution of (5-bromo-2-methoxy-phenyl)methanamine (10 g, 46.3 mmol) in MeOH (100 mL) under an atmosphere of N₂. The mixture was stirred at 25° C. for 12 h. LCMS showed desired MS was detected. The reaction mixture was filtered and concentrated under reduced pressure to give a residue. The residue was diluted with H₂O (50 mL) and extracted with DCM 200 mL (50 mL×4). The combined organic layers were washed with saturated aqueous NaCl solution (50 mL), dried over Na₂SO₄, filtered and concentrated under reduced pressure to give the title compound (12 g, crude) as a white solid which was used in the next step without further purification.

### Step 2: 5-Bromo-8-methoxyisoquinolin-3-amine

N-(5-Bromo-2-methoxybenzyl)-2,2-diethoxyacetimidamide (11 g, 31.9 mmol) was added to H₂SO₄ (111.61 g, 1.1 mol, 60.6 mL, 98% purity) at 0° C., and then the mixture was stirred at 25° C. for 3 h under an atmosphere of N₂. The reaction mixture was quenched by addition of NaOH (120 g) in H₂O (5 L) at 0° C. and extracted with 2-MeTHF (1L×2). The combined organic layers were washed with saturated aqueous NaCl solution (1 L), dried over Na₂SO₄, filtered and concentrated under reduced pressure to give a residue. The residue was purified by reverse phase prep-HPLC to give the title compound (3 g, 11.8 mmol, 37% yield) as a yellow solid.

### Step 3: 5-(1-Ethoxyvinyl)-8-methoxyisoquinolin-3-amine

Pd(PPh₃)₄(1.02 g, 884.8 μmol) and tributyl(1-ethoxyvinyl)stannane (6.79 g, 18.8 mmol, 6.3 mL) were added to a solution of 5-bromo-8-methoxyisoquinolin-3-amine (2.8 g, 11.1 mmol) in toluene (30 mL) under N₂ atmosphere. The mixture was stirred at 100° C. for 4 h under N₂ atmosphere. The reaction mixture was quenched by addition of aqueous CsF solution (60 mL) at 0° C. and extracted with EA (40 mL×3). The combined organic layers were washed with saturated aqueous NaCl solution (50 mL), dried over Na₂SO₄, filtered and concentrated under reduced pressure to give the title compound (2.7 g, crude) as a brown oil which was used in the next step without further purification.

### Step 4: 1-(3-Amino-8-methoxyisoquinolin-5-yl)ethan-1-one

A mixture of 5-(1-ethoxyvinyl)-8-methoxyisoquinolin-3-amine (2.7 g, 11.1 mmol) in THF (20 mL) and HCl (1M, 11 mL) was stirred at 25° C. for 30 min under N₂ atmosphere. The reaction mixture was filtered to give the title compound (1.1 g, crude) as a yellow solid which was used in the next step without further purification.

### Step 5: 1-(3-Chloro-8-methoxyisoquinolin-5-yl)ethan-1-one

Pyridine hydrochloride (1.03 g, 8.9 mmol), CuCl (25.2 mg, 254.3 umol, 6.08 uL) and NaNO₂ (614.2 mg, 8.9 mmol) were added to a solution of 1-(3-amino-8-methoxyisoquinolin-5-yl)ethan-1-one (550 mg, 2.5 mmol) in DCM (5 mL) under N₂ atmosphere. HCl (37.6 mg, 381.5 umol, 36.9 uL, 37% purity) was then added to the reaction mixture at −10° C., and the mixture was stirred at 0° C. for 30 min. The mixture was stirred at 25° C. for 1 h under N₂ atmosphere. The reaction mixture was filtered and concentrated under reduced pressure to give a residue. The residue was purified by flash-column chromatography to give the title compound (530 mg, 44% yield) as a white solid.

### Step 6: 2-(3-Chloro-8-methoxyisoquinolin-5-yl)propan-2-ol

MeMgBr (3M in THF, 1.41 mL) was added to a solution of 1-(3-Chloro-8-methoxyisoquinolin-5-yl)ethan-1-one (200 mg, 848.7 umol) in THF (2 mL) under N₂ atmosphere, and the reaction mixture was stirred at 25° C. for 1 h under N₂ atmosphere. The reaction mixture was quenched by the addition of H₂O (10 mL) at 0° C. The reaction mixture was filtered and concentrated under reduced pressure to give a residue. The residue was purified by flash-column chromatography to give the title compound (580 mg, 90.5% yield) as a yellow solid.

### Example 2k

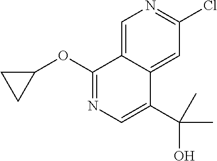 [SMILES: CC(C)(O)c1cnc(OC2CC2)c2cnc(Cl)cc12]

### Intermediate 30: 2-(6-Chloro-1-cyclopropoxy-2,7-naphthyridin-4-yl)propan-2-ol

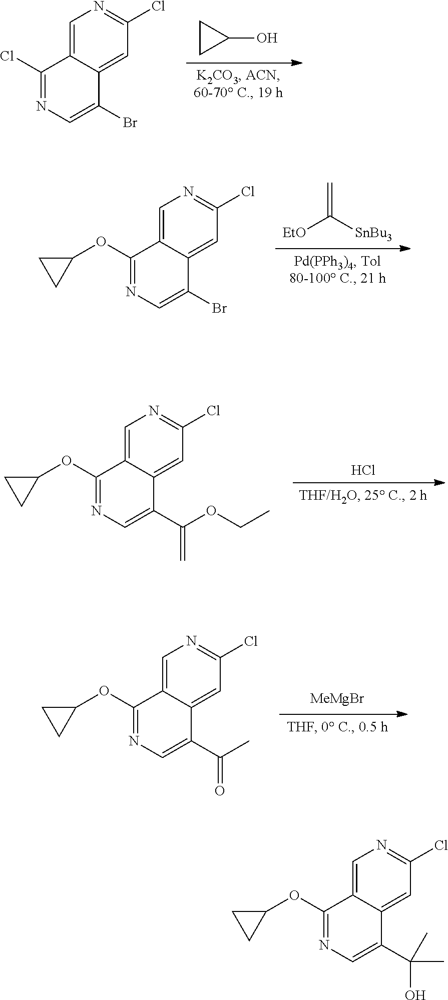

### Step 1: 4-Bromo-6-chloro-1-cyclopropoxy-2,7-naphthyridine

To a solution of 4-bromo-1,6-dichloro-2,7-naphthyridine (10.5 g, 37.78 mmol) and cyclopropanol (4.39 g, 75.6 mmol) in ACN (100 mL) was added K₂CO₃ (15.7 g, 113 mmol). The reaction mixture was stirred at 60° C. for 16 h, then additional cyclopropanol (4.39 g, 75.6 mmol) was added to the reaction mixture and it was heated to 70° C. for another 3 h. The reaction mixture was then concentrated to give a residue. The residue was diluted with water (200 mL), filtered and the filter cake was dried to give the title compound (11.3 g, 99% yield) as a yellow solid.

### Steps 2-3: 1-(6-Chloro-1-cyclopropoxy-2,7-naphthyridin-4-yl)ethan-1-one

The title compound was prepared from 4-bromo-6-chloro-1-cyclopropoxy-2,7-naphthyridine using a procedure similar to the that described in Steps 2-3 of Intermediate 20.

### Step 4: 2-(6-Chloro-1-cyclopropoxy-2,7-naphthyridin-4-yl)propan-2-ol

The title compound was prepared from 1-(6-chloro-1-cyclopropoxy-2,7-naphthyridin-4-yl)ethan-1-one using a procedure similar to the that described in Step 1 of Intermediate 22. MS (ES+) C₁₄H₁₅ClN₂O₂ requires: 278, found: 279[M+H]⁺.

### Example 21

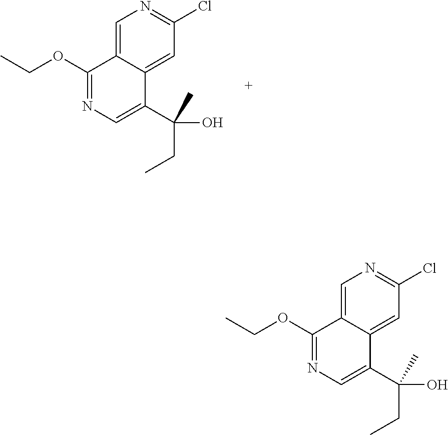 [SMILES: CCOc1ncc([C@](C)(O)CC)c2cc(Cl)ncc12 ; CCOc1ncc([C@@](C)(O)CC)c2cc(Cl)ncc12]

### Intermediates 31 and 32: (R)-2-(6-Chloro-1-ethoxy-2,7-naphthyridin-4-yl)butan-2-ol and (S)-2-(6-chloro-1-ethoxy-2,7-naphthyridin-4-yl)butan-2-ol

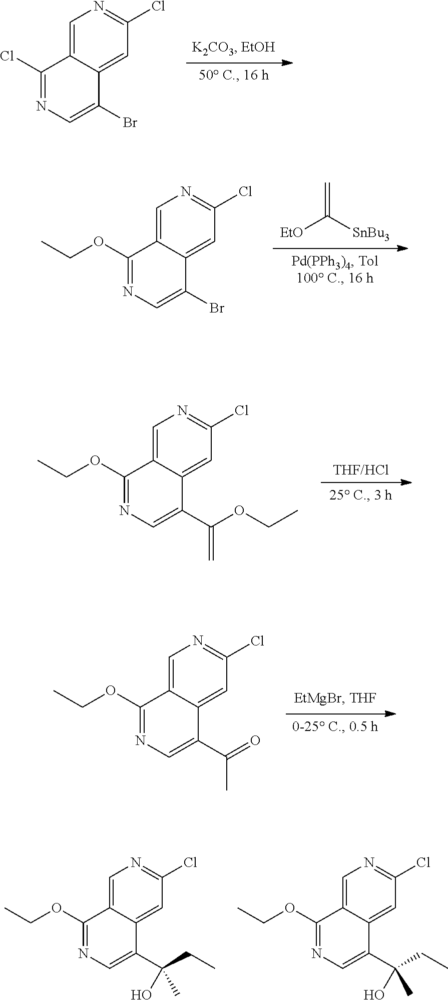

### Step 1: 4-Bromo-6-chloro-1-ethoxy-2,7-naphthyridine

A mixture of 4-bromo-1,6-dichloro-2,7-naphthyridine (3.00 g, 10.7 mmol) and K₂CO₃ (4.48 g, 32.3 mmol) in EtOH (30 mL) was stirred at 50° C. for 16 h, then was concentrated to give a residue. The residue was treated with water (100 mL), filtered, and the filter cake was dried to give the title compound (3.00 g, 97% yield) as white solid. MS (ES+) C₁₀H₈BrClN₂O requires: 288, found: 289[M+H]⁺.

### Steps 2-3: 1-(6-Chloro-1-ethoxy-2,7-naphthyridin-4-yl)ethan-1-one

The title compound was prepared from 4-bromo-6-chloro-1-ethoxy-2,7-naphthyridine using a procedure similar to the that described in Steps 2-3 of Intermediate 20.

### Step 4: (R)-2-(6-Chloro-1-ethoxy-2,7-naphthyridin-4-yl)butan-2-ol and (S)-2-(6-chloro-1-ethoxy-2,7-naphthyridin-4-yl)butan-2-ol

The racemate of the title compound was prepared from 1-(6-Chloro-1-ethoxy-2,7-naphthyridin-4-yl)ethan-1-one using a procedure similar to the that described in Step 4 of Intermediate 27. The mixture was separated by chiral SFC (column: Daicel Chiralpak AD (250 mm*30 mm, 10 um); mobile phase: [0.1% NH₃H₂O ETOH]; B %: 20%-20%) to give the first of (R)-2-(6-chloro-1-ethoxy-2,7-naphthyridin-4-yl)butan-2-ol or (S)-2-(6-chloro-1-ethoxy-2,7-naphthyridin-4-yl)butan-2-ol (peak 1, Intermediate 31, 50.0 mg, 50% yield) as yellow oil and the second one of (R)-2-(6-chloro-1-ethoxy-2,7-naphthyridin-4-yl)butan-2-ol or (S)-2-(6-chloro-1-ethoxy-2,7-naphthyridin-4-yl)butan-2-ol (peak 2, Intermediate 32, 50.0 mg, 50% yield) as yellow oil.

### Example 2m

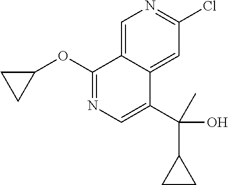 [SMILES: CC(O)(c1cnc(OC2CC2)c2cnc(Cl)cc12)C1CC1]

### Intermediate 33: 1-(6-Chloro-1-cyclopropoxy-2,7-naphthyridin-4-yl)-1-cyclopropylethan-1-ol

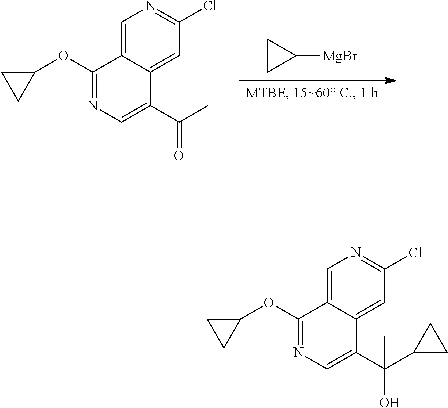

### Step 1: 1-(6-Chloro-1-cyclopropoxy-2,7-naphthyridin-4-yl)-1-cyclopropylethan-1-ol

To a solution of 1-(6-chloro-1-cyclopropoxy-2,7-naphthyridin-4-yl)ethan-1-one (500 mg, 1.90 mmol) in MTBE (8 mL) was added cyclopropylmagnesium bromide (3 M, 3.17 mL) at 15° C. The mixture was heated to 60° C. for 1 h, then was quenched by addition of saturated aqueous ammonium chloride solution (20 mL) and diluted with water (20 mL). The resulting mixture was extracted with EA (30 mL×3). The combined organic layers were dried over anhydrous sodium sulfate, filtered and concentrated to give a residue. The residue was purified by flash silica gel chromatography (Eluent of 0-65% EA/PE) to give the title compound (460 mg, 72% yield) as a white solid. MS (ES+) C₁₆H₁₇C₁N₂O₂ requires: 304, found: 305[M+H]⁺.

### Example 2m

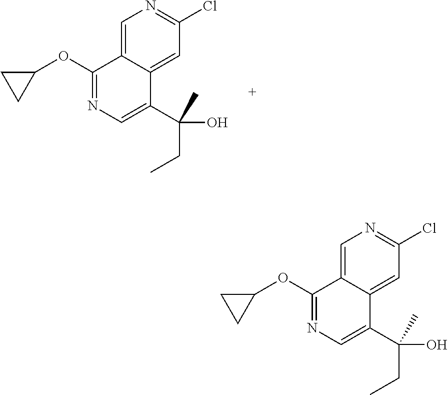 [SMILES: CC[C@@](C)(O)c1cnc(OC2CC2)c2cnc(Cl)cc12 ; "+" ; CC[C@](C)(O)c1cnc(OC2CC2)c2cnc(Cl)cc12]

### Intermediates 34 and 35: (R)-2-(6-Chloro-1-cyclopropoxy-2,7-naphthyridin-4-yl)butan-2-ol and (S)-2-(6-chloro-1-cyclopropoxy-2,7-naphthyridin-4-yl)butan-2-ol

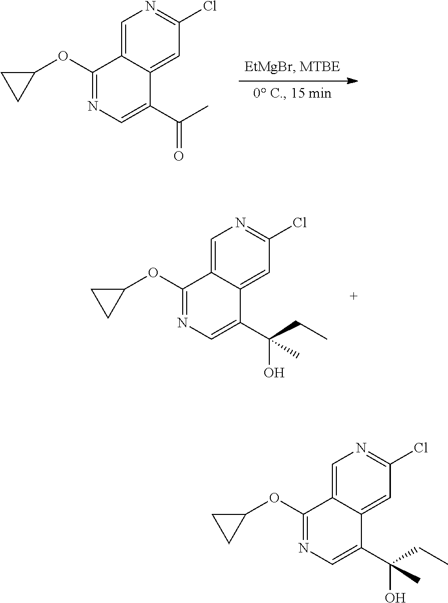 [SMILES: CC(=O)c1cnc(OC2CC2)c2cnc(Cl)cc12 ; "0° C., 15 min" ; "EtMgBr, MTBE" ; CC[C@@](C)(O)c1cnc(OC2CC2)c2cnc(Cl)cc12 ; CC[C@](C)(O)c1cnc(OC2CC2)c2cnc(Cl)cc12]

### Step 1: (R)-2-(6-Chloro-1-cyclopropoxy-2,7-naphthyridin-4-yl)butan-2-ol and (S)-2-(6-chloro-1-cyclopropoxy-2,7-naphthyridin-4-yl)butan-2-ol

To a solution of 1-(6-chloro-1-cyclopropoxy-2,7-naphthyridin-4-yl)ethan-1-one (300 mg, 1.14 mmol) in MTBE (10 mL) was added EtMgBr (3 M, 1.14 mL) at 0° C. The reaction mixture was stirred at 0° C. for 15 min, then was quenched with saturated aqueous ammonium chloride solution (20 mL) and extracted with EA (25 mL×3). The combined organic layers were dried over anhydrous sodium sulfate, filtered and concentrated to give a residue. The residue was purified by column chromatography (SiO₂, PE:EA=10:1 to 3:1) to give a racemic mixture of the title compounds (300 mg, 84% yield) as a yellow solid. ¹H-NMR (400 MHz, CDCl₃): δ ppm 9.31 (s, 1H), 8.55 (s, 1H), 8.22 (s, 1H), 4.55-4.47 (m, 1H), 2.13-2.05 (m, 2H), 1.75 (s, 3H), 0.94-0.91 (m, 4H), 0.90-0.85 (m, 3H). The mixture was separated by chiral SFC (column: Daicel Chiralpak IG (250 mm*30 mm, 10 um); mobile phase: [0.1% NH₃H₂O MeOH]; B %: 30%-30%) to give the first of (R)-2-(6-chloro-1-cyclopropoxy-2,7-naphthyridin-4-yl)butan-2-ol or (S)-2-(6-chloro-1-cyclopropoxy-2,7-naphthyridin-4-yl)butan-2-ol (peak 1, Intermediate 34, 90.0 mg, 60% yield) as a yellow solid and the second one of (R)-2-(6-chloro-1-cyclopropoxy-2,7-naphthyridin-4-yl)butan-2-ol or (S)-2-(6-chloro-1-cyclopropoxy-2,7-naphthyridin-4-yl)butan-2-ol (peak 2, Intermediate 35105 mg, 70% yield) as a yellow solid.

### Example 2n

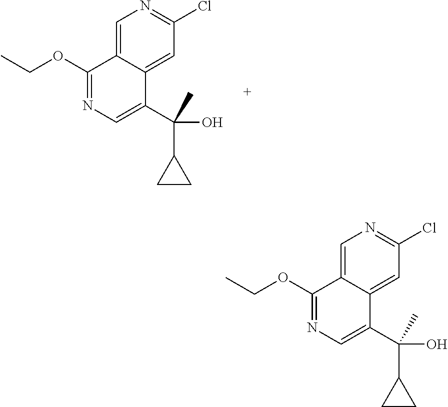 [SMILES: CCOc1ncc([C@](C)(O)C2CC2)c2cc(Cl)ncc12 ; "+" ; CCOc1ncc([C@@](C)(O)C2CC2)c2cc(Cl)ncc12]

### Intermediates 36 and 37: (R)-1-(6-Chloro-1-ethoxy-2,7-naphthyridin-4-yl)-1-cyclopropylethan-1-ol and (S)-1-(6-chloro-1-ethoxy-2,7-naphthyridin-4-yl)-1-cyclopropylethan-1-ol

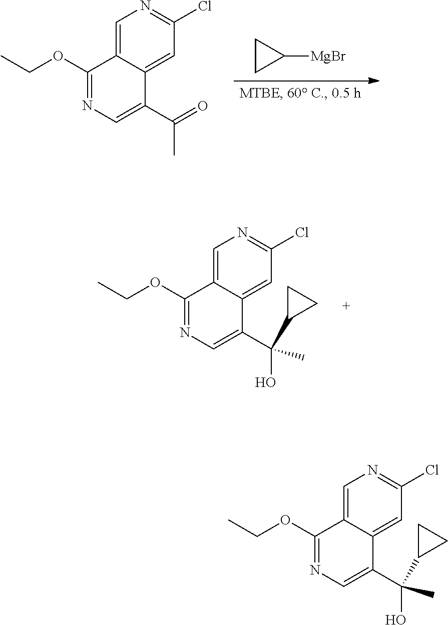

### Step 1: (R)-1-(6-Chloro-1-ethoxy-2,7-naphthyridin-4-yl)-1-cyclopropylethan-1-ol and (S)-1-(6-chloro-1-ethoxy-2,7-naphthyridin-4-yl)-1-cyclopropylethan-1-ol

To a solution of 1-(6-chloro-1-ethoxy-2,7-naphthyridin-4-yl)ethan-1-one (300 mg, 1.20 mmol) in MTBE (20 mL) was added cyclopropylmagnesium bromide (0.5 M, 11.9 mL). The mixture was stirred at 60° C. 0.5 h, then was quenched by addition of saturated aqueous ammonium chloride solution (30 mL) and extracted with EA (2×30 mL). The combined organic layers were dried over sodium sulfate, filtered, and concentrated to give a residue. The residue was purified by prep-TLC (PE/EA=1/1) to give a racemic mixture of the title compounds (80.0 mg, 23% yield) as a yellow oil. The mixture was separated by chiral SFC (column: Daicel Chiralpak IG (250 mm*30 mm, 10 um); mobile phase: [0.1% NH₃H₂O MEOH]; B %: 30%-30%) 4 min) to give the first one of (R)-1-(6-chloro-1-ethoxy-2,7-naphthyridin-4-yl)-1-cyclopropylethan-1-ol or (S)-1-(6-chloro-1-ethoxy-2,7-naphthyridin-4-yl)-1-cyclopropylethan-1-ol (peak 1, Intermediate 36, 40.0 mg, 50% yield) as a yellow solid and the second one of (R)-1-(6-chloro-1-ethoxy-2,7-naphthyridin-4-yl)-1-cyclopropylethan-1-ol or (S)-1-(6-chloro-1-ethoxy-2,7-naphthyridin-4-yl)-1-cyclopropylethan-1-ol (peak 2, Intermediate 37, 40.0 mg, 50% yield) as a yellow solid.

### Example 20

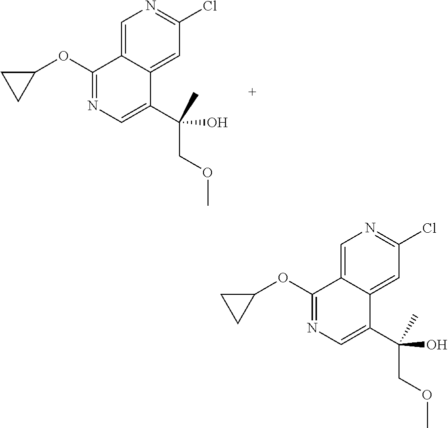 [SMILES: COC[C@@](C)(O)c1cnc(OC2CC2)c2cnc(Cl)cc12 ; "+" ; COC[C@](C)(O)c1cnc(OC2CC2)c2cnc(Cl)cc12]

### Intermediates 38 and 39: (S)-2-(6-Chloro-1-cyclopropoxy-2,7-naphthyridin-4-yl)-1-methoxypropan-2-ol and (R)-2-(6-chloro-1-cyclopropoxy-2,7-naphthyridin-4-yl)-1-methoxypropan-2-ol

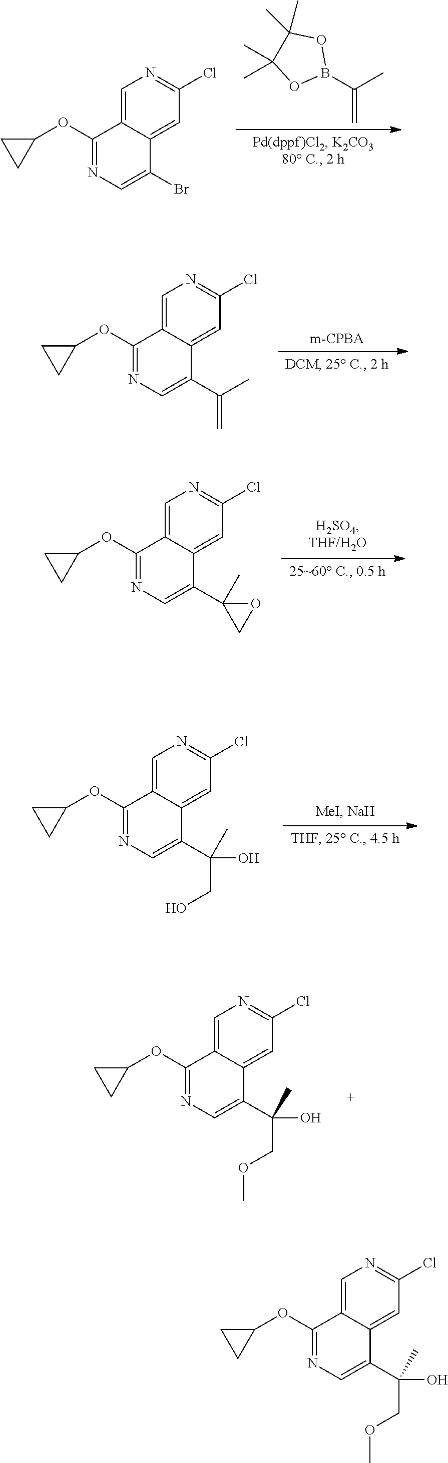

### Step 1: 6-Chloro-1-cyclopropoxy-4-(prop-1-en-2-yl)-2,7-naphthyridine

A mixture of 4-bromo-6-chloro-1-cyclopropoxy-2,7-naphthyridine (8 g, 26.7 mmol), 4,4,5,5-tetramethyl-2-(prop-1-en-2-yl)-1,3,2-dioxaborolane (4.71 g, 28.0 mmol), Pd(dppf)Cl₂ (1.95 g, 2.67 mmol) and K₂CO₃ (11.1 g, 80.1 mmol) in dioxane (150 mL) and H₂O (30 mL) was stirred at 80° C. for 2 h. The reaction mixture was then extracted with EA (100 mL), and the organic layer was washed with brine (100 mL), dried over Na₂SO₄, filtered and concentrated. The residue was purified by column chromatography on silica gel (PE/EA=10/1) to give the title compound (5.7 g, 78% yield) as a colorless oil.

### Step 2: 6-Chloro-1-cyclopropoxy-4-(2-methyloxiran-2-yl)-2,7-naphthyridine

To a solution of 6-chloro-1-cyclopropoxy-4-(prop-1-en-2-yl)-2,7-naphthyridine (5.7 g, 21.9 mmol) in DCM (80 mL) was added mCPBA (7.07 g, 32.8 mmol, 80% purity) in portions. The reaction mixture was stirred at 25° C. for 2 h, then was quenched with saturated aqueous Na₂SO₃ (100 mL) and extracted with EA (100 mL). The organic layer was dried over Na₂SO₄, filtered and concentrated. The residue was purified by column chromatography on silica gel (PE/EA=4/1) to give the title compound (2.2 g, 28% yield) as a white solid. MS (ES+) C₁₄H₁₃ClN₂O₂ requires: 276, found: 277[M+H]⁺.

### Step 3: 2-(6-Chloro-1-cyclopropoxy-2,7-naphthyridin-4-yl)propane-1,2-diol

To a solution of 6-chloro-1-cyclopropoxy-4-(2-methyloxiran-2-yl)-2,7-naphthyridine (2.2 g, 7.95 mmol) in THF (16 mL) was added a solution of H₂SO₄ (780 mg, 7.95 mmol) in H₂O (4 mL) dropwise at 25° C. The reaction mixture was stirred at 60° C. for 0.5 h, then was poured into saturated aqueous NaHCO₃ solution (50 mL) and extracted with EA (100 mL).

The organic layer was dried over Na₂SO₄, filtered and concentrated to give the title compound (2.1 g, 70% yield) as a yellow oil that was used in the next step without further purification. MS (ES+) C₁₄H₁₅ClN₂O₃ requires: 294, found: 295[M+H]⁺.

### Step 4: (S)-2-(6-Chloro-1-cyclopropoxy-2,7-naphthyridin-4-yl)-1-methoxypropan-2-ol and (R)-2-(6-chloro-1-cyclopropoxy-2,7-naphthyridin-4-yl)-1-methoxypropan-2-ol

To a solution of 2-(6-chloro-1-cyclopropoxy-2,7-naphthyridin-4-yl)propane-1,2-diol (500 mg, 1.33 mmol) in THF (10 mL) was added NaH (133 mg, 3.33 mmol, 60% purity) at 25° C. The reaction mixture was stirred at 25° C. for 0.5 h, and then CH₃I (198 mg, 1.40 mmol) was added. The reaction mixture was stirred at 25° C. for 4 h, then was filtered and concentrated. The residue was purified by prep-HPLC (column: Phenomenex luna C18 150*40 mm*15 um; mobile phase: [water (0.225% FA)—ACN]; B %: 35%-65%, 10 min) to give a racemic mixture of title compounds (100 mg, 23% yield) as a yellow oil. MS (ES+) C₁₅H₁₇ClN₂O₃ requires: 308, found: 309[M+H]⁺. The mixture was separated by chiral SFC (column: Daicel Chiralpak IG (250 mm*30 mm, 10 um); mobile phase: [0.1% NH₃H₂O—MEOH]; B %: 25%-25%) to give the first one of (S)-2-(6-chloro-1-cyclopropoxy-2,7-naphthyridin-4-yl)-1-methoxypropan-2-ol or (R)-2-(6-chloro-1-cyclopropoxy-2,7-naphthyridin-4-yl)-1-methoxypropan-2-ol (peak 1, Intermediate 38, 135 mg, 34% yield) as a yellow oil and the second one of (S)-2-(6-chloro-1-cyclopropoxy-2,7-naphthyridin-4-yl)-1-methoxypropan-2-ol or (R)-2-(6-chloro-1-cyclopropoxy-2,7-naphthyridin-4-yl)-1-methoxypropan-2-ol (peak 2, Intermediate 39, 25 mg, 25% yield).

### Example 2p

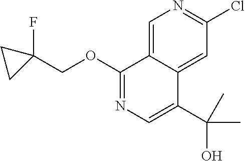 [SMILES: CC(C)(O)c1cnc(OCC2(F)CC2)c2cnc(Cl)cc12]

### Intermediate 40: 2-(6-Chloro-1-((1-fluorocyclopropyl)methoxy)-2,7-naphthyridin-4-yl)propan-2-ol

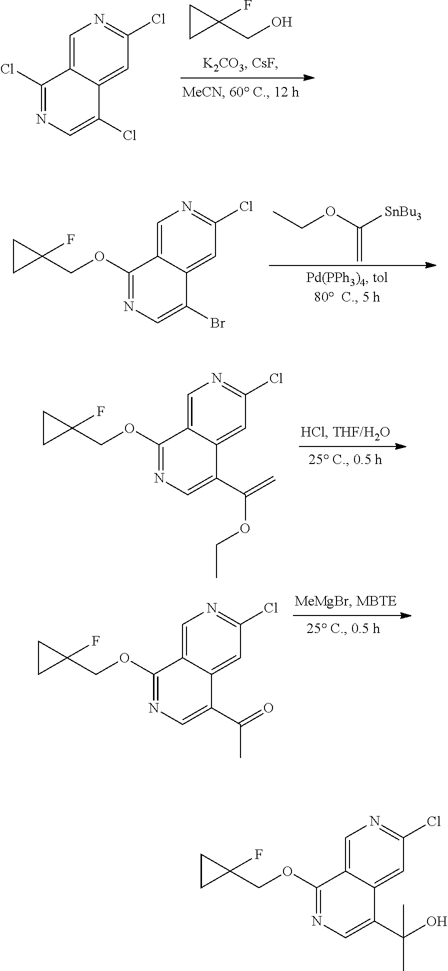

### Step 1: 4-Bromo-6-chloro-1-((1-fluorocyclopropyl)methoxy)-2,7-naphthyridine

To a solution of 4-bromo-1,6-dichloro-2,7-naphthyridine-1 (771 mg, 2.77 mmol) and (1-fluorocyclopropyl)methanol (500 mg, 5.55 mmol) in ACN (15 mL) was added K₂CO₃ (1.15 g, 8.32 mmol) and CsF (422 mg, 2.77 mmol). The reaction mixture was stirred at 60° C. for 12 h, then was concentrated. The residue was purified by silica gel column chromatography [PE:EA=10:1] to give the title compound (800 mg, 87% yield) as a colorless oil.

### Steps 2-3: 1-(6-Chloro-1-((1-fluorocyclopropyl)methoxy)-2,7-naphthyridin-4-yl)ethan-1-one

The title compound was prepared from 4-bromo-6-chloro-1-((1-fluorocyclopropyl) methoxy)-2,7-naphthyridine using a procedure similar to the that described in Steps 2-3 of Intermediate 20.

### Step 4: 2-(6-Chloro-1-((1-fluorocyclopropyl)methoxy)-2,7-naphthyridin-4-yl)propan-2-ol

To a solution of 1-(6-chloro-1-((1-fluorocyclopropyl)methoxy)-2,7-naphthyridin-4-yl)ethan-1-one (310 mg, 1.05 mmol) in MBTE (4.00 mL) was added MeMgBr (3 mol, 1.75 mL). The reaction mixture was stirred at 25° C. for 0.5 h, then was poured into saturated aqueous ammonium chloride solution (20 mL) and extracted with EA (40 mL×3). The combined organic layers were washed with water, brine, dried over anhydrous sodium sulfate, filtered, and concentrated. The residue was purified by silica gel column chromatography [PE:EA=20:1 to 2:1] to give the title compound (440 mg, crude) as a yellow oil.

### Example 2q

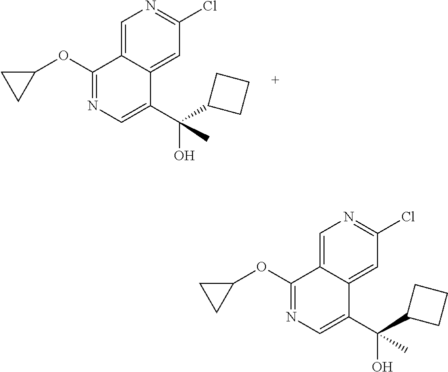

### Intermediates 41 and 42: (S)-1-(6-Chloro-1-cyclopropoxy-2,7-naphthyridin-4-yl)-1-cyclobutylethan-1-ol and (R)-1-(6-chloro-1-cyclopropoxy-2,7-naphthyridin-4-yl)-1-cyclobutylethan-1-ol

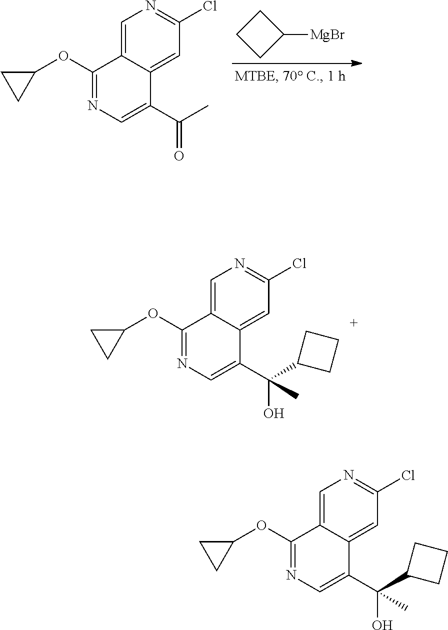

### Step 1: (S)-1-(6-Chloro-1-cyclopropoxy-2,7-naphthyridin-4-yl)-1-cyclobutylethan-1-ol (R)-1-(6-chloro-1-cyclopropoxy-2,7-naphthyridin-4-yl)-1-cyclobutylethan-1-ol

To a solution of 1-(6-chloro-1-cyclopropoxy-2,7-naphthyridin-4-yl)ethan-1-one (300 mg, 1.14 mmol) in MTBE (20 mL) was added cyclobutylmagnesium bromide (0.5 M, 7 mL) at 70° C. The reaction mixture was stirred at 70° C. for 1 h, then was quenched by addition of water (30 mL) at 20° C. The mixture was extracted with EA (30 mL×2), and the combined organic layers were washed with water (30 mL), dried over anhydrous sodium sulfate, filtered and concentrated to give a residue. The residue purified by prep-HPLC (column: Phenomenex luna C18 150×40 mm×15 um; mobile phase: [water (0.1% TFA)—ACN]; B %: 47%-77%, 11 min) to give a racemic mixture of the title compounds (110 mg, 27% yield) as a yellow oil. The mixture was separated by chiral SFC (column: Daicel Chiralpak IG (250 mm×30 mm, 10 um); mobile phase: [0.1% NH₃H₂O MEOH]; B %: 30%-30%) to give the first one of (S)-1-(6-chloro-1-cyclopropoxy-2,7-naphthyridin-4-yl)-1-cyclobutylethan-1-ol or (R)-1-(6-chloro-1-cyclopropoxy-2,7-naphthyridin-4-yl)-1-cyclobutylethan-1-ol (peak 1, Intermediate 41, 30.0 mg, 27% yield) as a yellow solid and the second one of (S)-1-(6-chloro-1-cyclopropoxy-2,7-naphthyridin-4-yl)-1-cyclobutylethan-1-ol or (R)-1-(6-chloro-1-cyclopropoxy-2,7-naphthyridin-4-yl)-1-cyclobutylethan-1-ol (peak 2, Intermediate 42, 30.0 mg, 27% yield) as a yellow solid.

### Example 2r

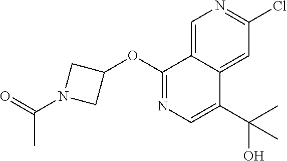 [SMILES: CC(=O)N1CC(Oc2ncc(C(C)(C)O)c3cc(Cl)ncc23)C1]

### Intermediate 43: 1-(3-((6-chloro-4-(2-hydroxypropan-2-yl)-2,7-naphthyridin-1-yl)oxy)azetidin-1-yl)ethan-1-one

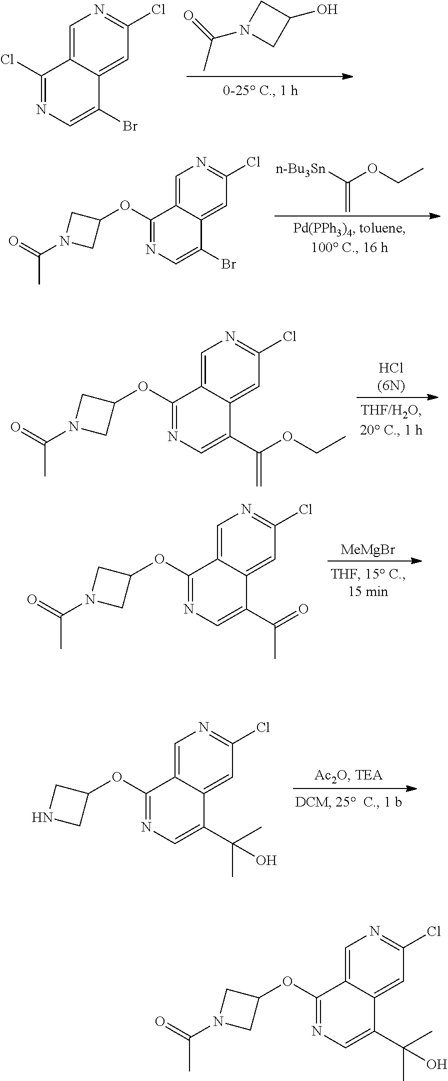

### Step 1: 1-(3-((4-Bromo-6-chloro-2,7-naphthyridin-1-yl)oxy)azetidin-1-yl)ethan-1-one

Sodium hydride (2.15 g, 53.8 mmol, 60 wt %) was added to a solution of 1-(3-hydroxyazetidin-1-yl)ethan-1-one (Combi-Blocks #ST-8959) (5.16 g, 44.8 mmol) in THF (200 mL) at 0° C. The cooling bath was removed, the reaction mixture was stirred for 0.5 h, and then 4-bromo-1,6-dichloro-2,7-naphthyridine (12.4 g, 44.8 mmol) was added. The reaction mixture was stirred at 25° C. for 1 h in MeOH, then was quenched by addition of water (70 mL). The quenched reaction mixture was extracted with EA, dried over sodium sulfate, filtered, and concentrated to give the title compound (10.0 g, 63% yield).

### Step 2: 1-(3-((6-Chloro-4-(1-ethoxyvinyl)-2,7-naphthyridin-1-yl)oxy)azetidin-1-yl)ethan-1-one

A mixture of compound tributyl(1-ethoxyvinyl)stannane (9.14 g, 25.3 mmol 1-(3-((4-bromo-6-chloro-2,7-naphthyridin-1-yl)oxy)azetidin-1-yl)ethan-1-one (8.20 g, 23.0 mmol) and Pd(PPh₃)₄(2.66 g, 2.30 mmol) in toluene (150 mL) was degassed and purged with nitrogen 3 times, and then the mixture was stirred at 100° C. for 16 h. The reaction mixture was cooled to ambient temperature, quenched by addition aqueous saturated KF (300 mL), diluted with water (100 mL), and extracted with EA (200 mL×3). The combined organic layers were dried over anhydrous sodium sulfate, filtered and concentrated to give a residue. The residue was purified by flash-column chromatography on silica gel to give the title compound (7.50 g, 94% yield) as a yellow solid.

### Step 3: 1-(3-((4-Acetyl-6-chloro-2,7-naphthyridin-1-yl)oxy)azetidin-1-yl)ethan-1-one

Aqueous hydrochloric acid (6 M, 0.5 mL) was added to a solution of 1-(3-((6-chloro-4-(1-ethoxyvinyl)-2,7-naphthyridin-1-yl)oxy)azetidin-1-yl)ethan-1-one (6.90 g, 19.8 mmol) in THF (105 mL) and water (35 mL) at 20° C. The reaction mixture was stirred at 20° C. for 1 h, then was diluted with water (300 mL) and extracted with EA (200 mL×3). The combined organic layers were dried over sodium sulfate, filtered and concentrated. The residue was purified by flash-column chromatography on silica gel (gradient elution, 0% to 100% EA-PE) to give the title compound (5.00 g, 79% yield) as a yellow solid.

### Steps 4 and 5: 1-(3-((6-Chloro-4-(2-hydroxypropan-2-yl)-2,7-naphthyridin-1-yl)oxy)azetidin-1-yl)ethan-1-one

Methylmagnesium bromide (3 M in diethyl ether, 14 mL) was added to a solution of 1-(3-((4-acetyl-6-chloro-2,7-naphthyridin-1-yl)oxy)azetidin-1-yl)ethan-1-one (4.50 g, 14.1 mmol) in THF (200 mL) at 0° C. The reaction mixture was stirred at 0° C. for 15 min, then was quenched with water (200 mL) and extracted with EA (150 mL×3). The combined organic layers were dried over sodium sulfate, filtered and concentrated to give a mixture of 1-(3-((6-chloro-4-(2-hydroxypropan-2-yl)-2,7-naphthyridin-1-yl)oxy)azetidin-1-yl)ethan-1-one and 2-(1-(azetidin-3-yloxy)-6-chloro-2,7-naphthyridin-4-yl)propan-2-ol (4.10 g, 99% yield) as a yellow oil. The mixture was dissolved in DCM (50 mL) and TEA (3.53 g, 34.9 mmol) and acetic anhydride (2.14 g, 20.9 mmol) were added. The reaction mixture was stirred at 25° C. for 1 h, then was diluted with water (200 mL) and extracted with DCM (150 mL×3). The combined organic layers were dried over sodium sulfate, filtered and concentrated to give a residue. The residue was purified by flash-column chromatography on silica gel (gradient elution, 50% to 100% EA-PE, then 5% to 10% MeOH-EA) to give the title compound (4.10 g, 87% yield) as a yellow oil.

### Example 2s

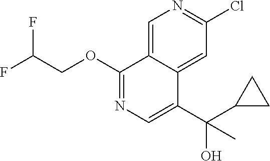 [SMILES: CC(O)(c1cnc(OCC(F)F)c2cnc(Cl)cc12)C1CC1]

### Intermediate 44: 1-(6-Chloro-1-(2,2-difluoroethoxy)-2,7-naphthyridin-4-yl)-1-cyclopropylethan-1-ol

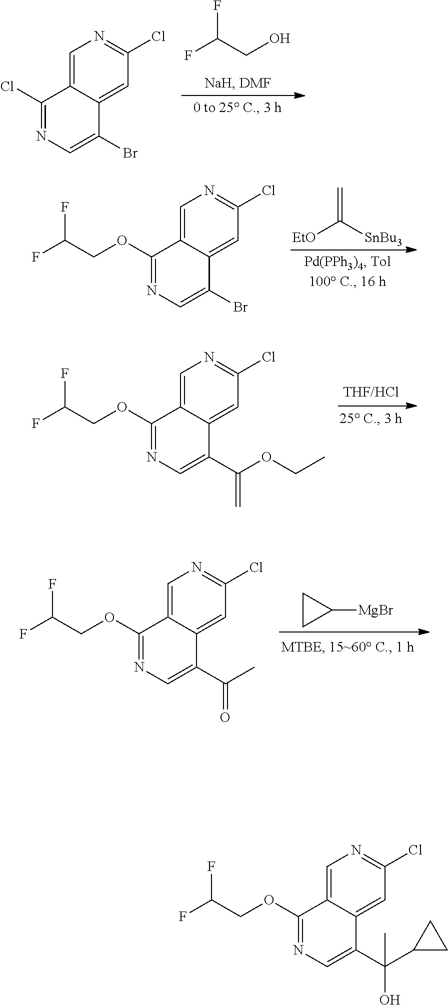

### Step 1: 4-Bromo-6-chloro-1-(2,2-difluoroethoxy)-2,7-naphthyridine

NaH (60 wt %, 0.863 g, 21.6 mmol) was added to a solution of 2,2-difluoroethan-1-ol (1.624 g, 19.79 mmol) in DMF (36 mL) at 0° C. The reaction mixture was stirred at 0° C. for 30 min, then 4-bromo-1,6-dichloro-2,7-naphthyridine (5.0 g, 18 mmol) was added in one portion. The reaction mixture was removed from the cooling bath and stirred at 25° C. for 3 h. The reaction mixture was then diluted with EA and washed with saturated aqueous sodium bicarbonate solution, brine, dried over sodium sulfate, filtered, and concentrated. The residue was purified by flash-column chromatography on silica gel (20-100% EA-Hexanes) to give the title compound (2.1 g, 36% yield) as a yellow solid.

### Steps 2-3: 1-(6-Chloro-1-(2,2-difluoroethoxy)-2,7-naphthyridin-4-yl)ethan-1-one

The title compound was prepared from 4-bromo-6-chloro-1-(2,2-difluoroethoxy)-2,7-naphthyridine using a procedure similar to the that described in Steps 2-3 of Intermediate 20.

### Step 4: 1-(6-Chloro-1-(2,2-difluoroethoxy)-2,7-naphthyridin-4-yl)-1-cyclopropylethan-1-ol

Cyclopropylmagnesium bromide (0.5 M, 3.49 mL, 1.74 mmol) was added to a solution of 1-(6-chloro-1-(2,2-difluoroethoxy)-2,7-naphthyridin-4-yl)ethan-1-one (0.25 g, 0.87 mmol) at 25° C. The reaction mixture was stirred at 25° C. for 30 min, then was diluted with EA and washed with saturated aqueous sodium bicarbonate solution, brine, dried over anhydrous sodium sulfate, filtered and concentrated. The residue was purified by flash-column chromatography on silica gel (20-100% EA:Hexanes) to give the title compound (106 mg, 37.0% yield). MS (ES+) C₁₅H₁₅ClF₂N₂O₂ requires: 328, found: 329[M+H]⁺.

### Example 2t

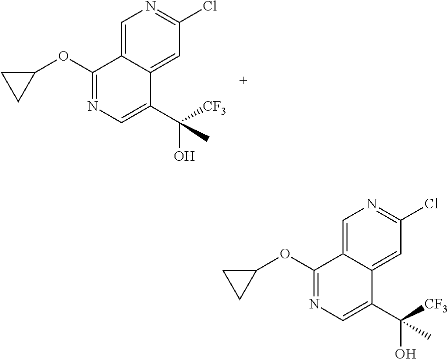

### Intermediates 45 and 46: (R)-2-(6-Chloro-1-cyclopropoxy-2,7-naphthyridin-4-yl)-1,1,1-trifluoropropan-2-ol and (S)-2-(6-chloro-1-cyclopropoxy-2,7-naphthyridin-4-yl)-1,1,1-trifluoropropan-2-ol

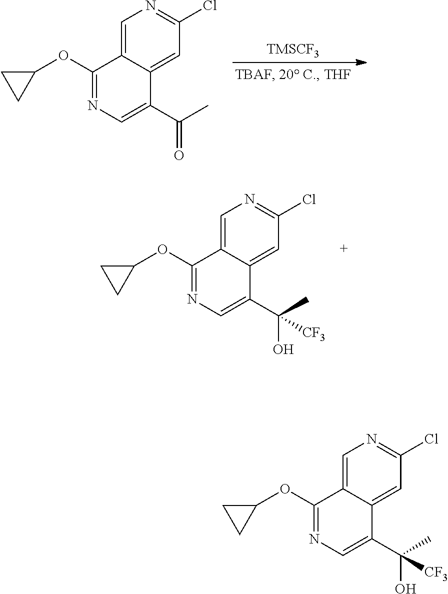 [SMILES: "TMSCF3" ; CC(=O)c1cnc(OC2CC2)c2cnc(Cl)cc12 ; C[C@@](O)(c1cnc(OC2CC2)c2cnc(Cl)cc12)C(F)(F)F ; C[C@](O)(c1cnc(OC2CC2)c2cnc(Cl)cc12)C(F)(F)F]

### Step 1: (R)-2-(6-Chloro-1-cyclopropoxy-2,7-naphthyridin-4-yl)-1,1,1-trifluoropropan-2-ol and (S)-2-(6-chloro-1-cyclopropoxy-2,7-naphthyridin-4-yl)-1,1,1-trifluoropropan-2-ol

To a solution of 1-(6-chloro-1-cyclopropoxy-2,7-naphthyridin-4-yl)ethan-1-one (300 mg, 1.14 mmol) and TMSCF₃ (244 mg, 1.71 mmol) in THF (20 mL) was added TBAF (1 M, 228 uL). The reaction mixture was stirred at 20° C. for 15 min, then was quenched by addition of water (30 mL) and extracted with EA (30 mL×2). The combined organic layers were washed with water (30 mL), dried over anhydrous sodium sulfate, filtered and concentrated. The residue was purified by prep-HPLC (column: Waters Xbridge 150*25 mm*5 um; mobile phase: [water (0.05% ammonium hydroxide v/v)—ACN]; B %: 41%-61%, 10 min) to give a racemic mixture of the title compounds (250 mg, 65% yield) as a yellow solid. ¹H-NMR (400 MHz, CDCl₃): δ ppm 9.24 (s, 1H), 8.52 (s, 1H), 8.26 (s, 1H), 4.50-4.45 (m, 1H), 2.88 (s, 1H), 1.93 (s, 3H), 0.86 (d, J=5.2 Hz, 4H). The mixture was separated by chiral SFC (column: Daicel Chiralpak OJ-H (250 mm*30 mm, 5 um); mobile phase: [0.1% NH₃H₂O IPA]; B %: 15%-15%) to give the first one of (R)-2-(6-chloro-1-cyclopropoxy-2,7-naphthyridin-4-yl)-1,1,1-trifluoropropan-2-ol or (S)-2-(6-chloro-1-cyclopropoxy-2,7-naphthyridin-4-yl)-1,1,1-trifluoropropan-2-ol (peak 1, Intermediate 45, 100 mg, 83% yield) and the second one of (R)-2-(6-chloro-1-cyclopropoxy-2,7-naphthyridin-4-yl)-1,1,1-trifluoropropan-2-ol or (S)-2-(6-chloro-1-cyclopropoxy-2,7-naphthyridin-4-yl)-1,1,1-trifluoropropan-2-ol (peak 2, Intermediate 46, 110 mg, 92% yield) as a yellow solid.

### Example 2u

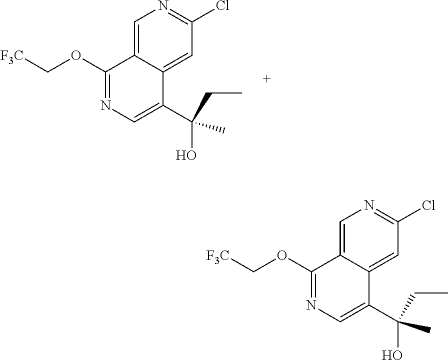 [SMILES: CC[C@@](C)(O)c1cnc(OCC(F)(F)F)c2cnc(Cl)cc12 ; CC[C@](C)(O)c1cnc(OCC(F)(F)F)c2cnc(Cl)cc12]

### Intermediates 47 and 48: (R)-2-(6-Chloro-1-(2,2,2-trifluoroethoxy)-2,7-naphthyridin-4-yl)butan-2-ol and (S)-2-(6-chloro-1-(2,2,2-trifluoroethoxy)-2,7-naphthyridin-4-yl)butan-2-ol

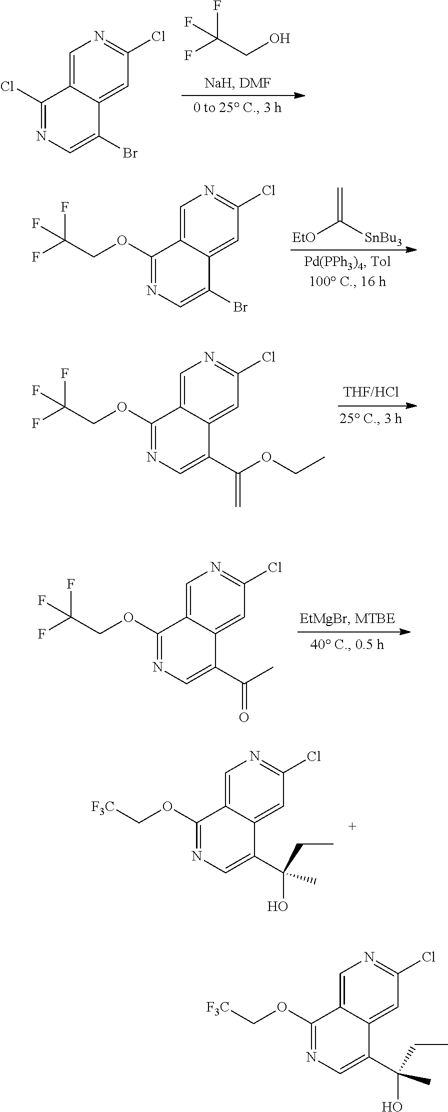

### Steps 1-3: 1-(6-Chloro-1-(2,2,2-trifluoroethoxy)-2,7-naphthyridin-4-yl)ethan-1-one

The title compound was prepared from 4-bromo-1,6-dichloro-2,7-naphthyridine and 2,2,2-trifluoroethan-1-ol using a similar procedure as described in Step 1 of Intermediate 44 and Steps 2-3 of Intermediate 20.

### Step 4: (R)-2-(6-Chloro-1-(2,2,2-trifluoroethoxy)-2,7-naphthyridin-4-yl)butan-2-ol and (S)-2-(6-chloro-1-(2,2,2-trifluoroethoxy)-2,7-naphthyridin-4-yl)butan-2-ol

To a solution of 1-(6-chloro-1-(2,2,2-trifluoroethoxy)-2,7-naphthyridin-4-yl)ethan-1-one (300 mg, 984 umol) in MTBE (10 mL) was added EtMgBr (3 M, 1.64 mL) at 40° C. The reaction mixture was stirred at 40° C. for 0.5 h, then was quenched by addition of saturated aqueous ammonium chloride solution (30 mL) and extracted with EA (3×30 mL). The combined organic layers were dried over anhydrous sodium sulfate, filtered and concentrated to give a residue. The residue was purified by prep-HPLC column (Phenomenex Gemini-NX C18 75*30 mm*3 um; mobile phase: [water (0.1% TFA)—ACN]; B %: 55%-65%, 7 min) to give a racemic mixture of the title compounds (50.0 mg, 15% yield) as a yellow solid. The mixture was separated by chiral SFC (Column: Chiralpak AD-3 50×4.6 mm I.D., 3 um; Mobile phase: Phase A for CO₂, and Phase B for EtOH (0.05% DEA); Gradient elution: B in A from 5% to 40%) to give the first one of (R)-2-(6-chloro-1-(2,2,2-trifluoroethoxy)-2,7-naphthyridin-4-yl)butan-2-ol or (S)-2-(6-chloro-1-(2,2,2-trifluoroethoxy)-2,7-naphthyridin-4-yl)butan-2-ol (peak 1, Intermediate 47, 25 mg, 50% yield) as a yellow oil and the second one of (R)-2-(6-chloro-1-(2,2,2-trifluoroethoxy)-2,7-naphthyridin-4-yl)butan-2-ol or (S)-2-(6-chloro-1-(2,2,2-trifluoroethoxy)-2,7-naphthyridin-4-yl)butan-2-ol (peak 2, Intermediate 48, 25 mg, 50% yield) as a yellow oil.

### Example 2v

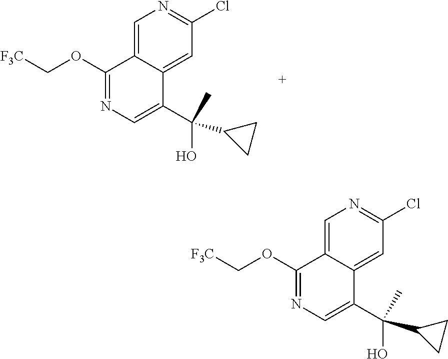 [SMILES: C[C@@](O)(c1cnc(OCC(F)(F)F)c2cnc(Cl)cc12)C1CC1 ; C[C@](O)(c1cnc(OCC(F)(F)F)c2cnc(Cl)cc12)C1CC1]

### Intermediates 49 and 50: (S)-1-(6-Chloro-1-(2,2,2-trifluoroethoxy)-2,7-naphthyridin-4-yl)-1-cyclopropylethan-1-ol and (R)-1-(6-chloro-1-(2,2,2-trifluoroethoxy)-2,7-naphthyridin-4-yl)-1-cyclopropylethan-1-ol

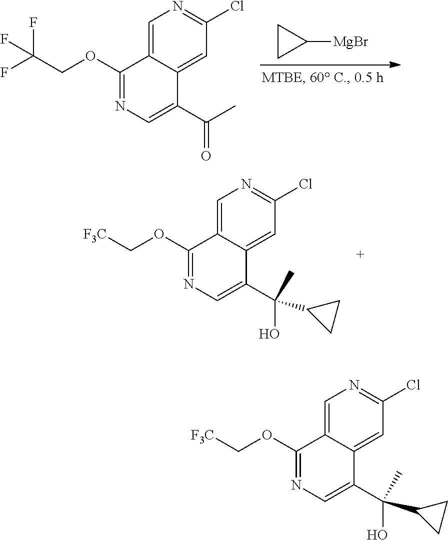

### Step 1: (S)-1-(6-Chloro-1-(2,2,2-trifluoroethoxy)-2,7-naphthyridin-4-yl)-1-cyclopropylethan-1-ol and (R)-1-(6-chloro-1-(2,2,2-trifluoroethoxy)-2,7-naphthyridin-4-yl)-1-cyclopropylethan-1-ol

A mixture of 1-(6-chloro-1-(2,2,2-trifluoroethoxy)-2,7-naphthyridin-4-yl)ethan-1-one (200 mg, 656 μmol) in MTBE (5 mL) was degassed and purged with nitrogen 3 times, and then cyclopropylmagnesium bromide (0.5 M, 2.63 mL) was added. The reaction mixture was stirred at 60° C. for 0.5 h, then was quenched by addition of saturated aqueous ammonium chloride solution (30 mL) at 25° C. and extracted with EA (30 mL×3). The combined organic layers were dried over anhydrous sodium sulfate, filtered and concentrated to give a residue. The residue was purified by column chromatography (SiO₂, PE/EA=1/0 to 5/1) to give a racemic mixture of the title compound (90.0 mg, 40% yield) as a yellow solid. MS (ES+) C₁₅H₁₄ClF₃N₂O₂ requires: 346, found: 347[M+H]⁺. The racemic mixture was separated by chiral SFC (column: Daicel Chiralpak AD-H (250 mm*30 mm, Sum); mobile phase: [0.1% NH₃H₂O IPA]; B %: 20%-20%) to give the first one of (S)-1-(6-chloro-1-(2,2,2-trifluoroethoxy)-2,7-naphthyridin-4-yl)-1-cyclopropylethan-1-ol or (R)-1-(6-chloro-1-(2,2,2-trifluoroethoxy)-2,7-naphthyridin-4-yl)-1-cyclopropylethan-1-ol (peak 1, Intermediate 49, 35.0 mg, 39% yield) as a white oil and the second one of (S)-1-(6-chloro-1-(2,2,2-trifluoroethoxy)-2,7-naphthyridin-4-yl)-1-cyclopropylethan-1-ol or (R)-1-(6-chloro-1-(2,2,2-trifluoroethoxy)-2,7-naphthyridin-4-yl)-1-cyclopropylethan-1-ol (peak 2, Intermediate 50, 45.0 mg, 50% yield) as a white oil. MS (ES+) C₁₅H₁₄C₁F₃N₂O₂ requires: 346, found: 347[M+H]⁺.

### Example 2w

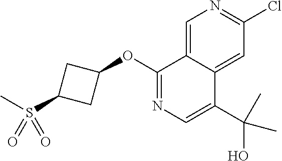 [SMILES: CC(C)(O)c1cnc(O[C@H]2C[C@@H](S(C)(=O)=O)C2)c2cnc(Cl)cc12]

### Intermediate 51: 2-(6-Chloro-1-(cis-3-met ylsulfonyl)cyclobutoxy)-2,7-naphthyridin-4-yl)propan-2-ol

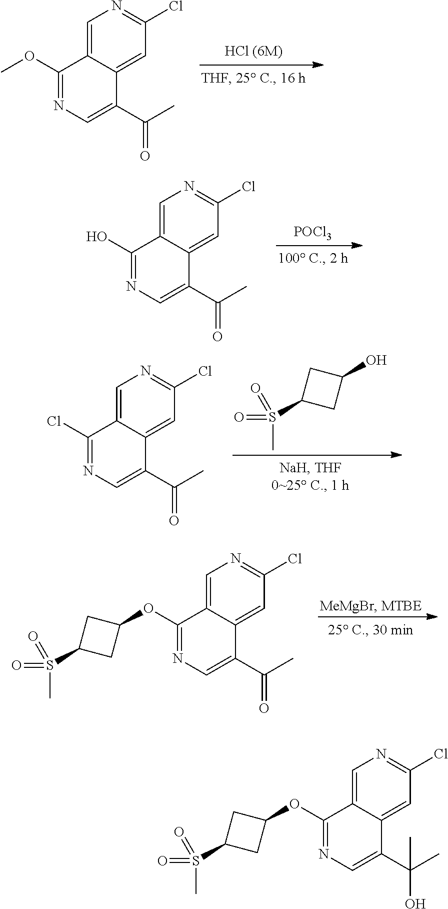

### Step 1: 1-(6-Chloro-1-hydroxy-2,7-naphthyridin-4-yl)ethan-1-one

To a solution of 1-(6-chloro-1-methoxy-2,7-naphthyridin-4-yl)ethan-1-one (3.00 g, 12.7 mmol) in THF (30 mL) was added aqueous HCl (6 M, 20 mL). The reaction mixture was stirred at 25° C. for 16 h, then was diluted with water (80 mL) and extracted with EA (60 mL×3). The combined organic layers were dried over anhydrous sodium sulfate, filtered and concentrated to give the title compound (2.80 g, 99% yield) as a white solid that was used in the next step without further purification. ¹H NMR (400 MHz, 6d-DMSO): δ ppm 12.45 (s, 1H), 9.16 (s, 1H), 8.80 (s, 1H), 8.45 (d, J=3.2 Hz, 1H), 2.53 (s, 3H).

### Step 2: 1-(1,6-Dichloro-2,7-naphthyridin-4-yl)ethan-1-one

1-(6-Chloro-1-hydroxy-2,7-naphthyridin-4-yl)ethan-1-one (1.00 g, 4.49 mmol) was added to POCl₃ (10 mL), and the reaction mixture was stirred at 100° C. and stirred for 2 h. The reaction mixture was then cooled to 23° C. and slowly poured into saturated aqueous sodium bicarbonate solution (500 mL). The mixture was extracted with EA (200 mL×3). The combined organic layers were dried over anhydrous sodium sulfate, filtered and concentrated. The residue was purified by silica gel column chromatography (PE:EA=1:0 to 3:1) to give the title compound (660 mg, 58% yield) as a white solid. ¹H NMR (400 MHz, CDCl₃): δ ppm 9.62 (s, 1H), 9.01 (s, 1H), 8.95 (s, 1H), 2.78 (s, 3H).

### Step 3: 1-(6-Chloro-1-(cis-3-(methylsulfonyl)cyclobutoxy)-2,7-naphthyridin-4-yl)ethan-1-one

To a solution of cis-3-(methylsulfonyl)cyclobutan-1-ol (685 mg, 4.56 mmol) in THF (10 mL) was added NaH (199 mg, 4.98 mmol, 60% purity) at 0° C. The reaction mixture was stirred at 25° C. for 0.5 h, then 1-(1,6-dichloro-2,7-naphthyridin-4-yl)ethan-1-one (1.00 g, 4.15 mmol) was added and the mixture was stirred at 25° C. for 0.5 h. The reaction mixture was then diluted with saturated aqueous sodium chloride solution (50 mL) and extracted with EA (50 mL×3). The combined organic layers were dried over anhydrous sodium sulfate, filtered and concentrated to give a residue. The residue was purified by column chromatography (SiO₂, PE/EA=1:0 to 1:4) to give the title compound (1.05 g, 2.96 mmol, 71% yield) as a yellow solid. MS (ES+) C₁₅H₁₅ClN₂O₄S requires: 354, found: 355[M+H]⁺.

### Step 4: 2-(6-Chloro-1-(cis-3-(methylsulfonyl)cyclobutoxy)-2,7-naphthyridin-4-yl)propan-2-ol

To a solution of 1-(6-chloro-1-(cis-3-(methylsulfonyl)cyclobutoxy)-2,7-naphthyridin-4-yl)ethan-1-one (350 mg, 986 μmol) in THF (10 mL) was added MeMgBr (3 M, 1.64 mL) at 25° C. The reaction mixture was stirred at 25° C. for 30 min, then was diluted with saturated aqueous ammonium chloride solution (25 mL) and extracted with EA (25 mL×3). The combined organic layers were dried over anhydrous sodium sulfate, filtered and concentrated to give a residue. The residue was purified by column chromatography (SiO₂, PE/EA=0:1 to 1:3) to give the title compound (170 mg, 46% yield) as a yellow solid. MS (ES+) C₁₆H₁₉ClN₂O₄S requires: 370, found: 371[M+H]⁺.

### Example 2x

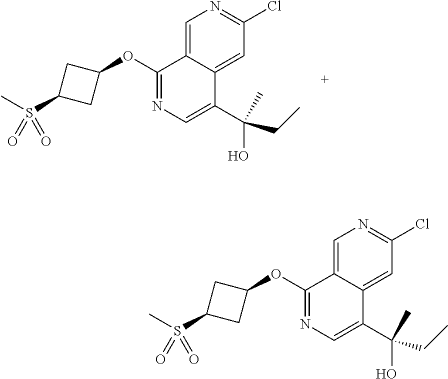

### Intermediates 52 and 53: (R)-2-(6-Chloro-1-(cis-3-(methylsulfonyl)cyclobutoxy)-2,7-naphthyridin-4-yl)butan-2-ol and (S)-2-(6-chloro-1-(cis-3-(methylsulfonyl)cyclobutoxy)-2,7-naphthyridin-4-yl)butan-2-ol

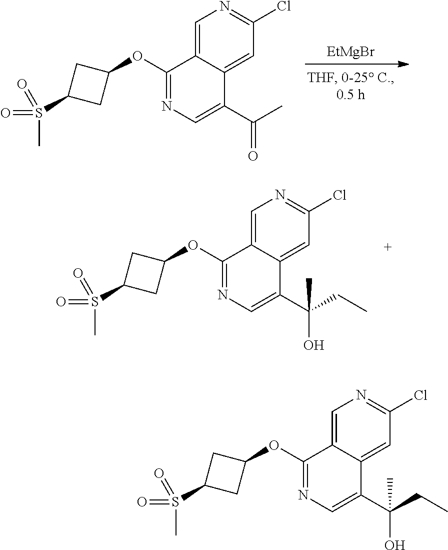 [SMILES: "EtMgBr" ; CC(=O)c1cnc(O[C@H]2C[C@@H](S(C)(=O)=O)C2)c2cnc(Cl)cc12 ; CC[C@](C)(O)c1cnc(O[C@H]2C[C@@H](S(C)(=O)=O)C2)c2cnc(Cl)cc12 ; CC[C@@](C)(O)c1cnc(O[C@H]2C[C@@H](S(C)(=O)=O)C2)c2cnc(Cl)cc12]

### Step 1: (R)-2-(6-Chloro-1-(cis-3-(methylsulfonyl)cyclobutoxy)-2,7-naphthyridin-4-yl)butan-2-ol and (S)-2-(6-chloro-1-(cis-3-(methylsulfonyl)cyclobutoxy)-2,7-naphthyridin-4-yl)butan-2-ol

To a solution of 1-(6-chloro-1-(cis-3-(methylsulfonyl)cyclobutoxy)-2,7-naphthyridin-4-yl)ethan-1-one (650 mg, 1.83 mmol, 1 eq) in THF (10 mL) was added EtMgBr (3 M, 1.83 mL, 3 eq) at 0° C. The cooling bath was removed, and the reaction mixture was stirred at 25° C. for 15 min. The reaction mixture was quenched by addition of water (50 mL) and extracted with EA (50 mL×3). The organic layers were dried over anhydrous sodium sulfate, filtered and concentrated to give a residue. The residue was purified by column chromatography (SiO₂, PE/EA=1/0 to 0/1) followed by prep-HPLC (column: Phenomenex Gemini-NX C18 75*30 mm*3 um; mobile phase: [water (0.225% FA)—ACN]; B %: 28%-58%, 7 min) to give a racemic mixture of the title compounds (110 mg, 15.6% yield) as a yellow solid. The mixture was separated by chiral SFC (Column: Chiralpak IG-3 50×4.6 mm I.D., 3 um Mobile phase: Phase A for CO₂, and Phase B for MeOH (0.05% DEA); Gradient elution: 40% MeOH (0.05% DEA) in CO₂) to give the first one of (R)-2-(6-chloro-1-(cis-3-(methylsulfonyl)cyclobutoxy)-2,7-naphthyridin-4-yl)butan-2-ol or (S)-2-(6-chloro-1-(cis-3-(methylsulfonyl)cyclobutoxy)-2,7-naphthyridin-4-yl)butan-2-ol (peak 1, Intermediate 52, 40 mg) as a yellow solid and the second one of (R)-2-(6-chloro-1-(cis-3-(methylsulfonyl)cyclobutoxy)-2,7-naphthyridin-4-yl)butan-2-ol or (S)-2-(6-chloro-1-(cis-3-(methylsulfonyl)cyclobutoxy)-2,7-naphthyridin-4-yl)butan-2-ol (peak 2, Intermediate 53, 40 mg) as a yellow solid. MS (ES+) C₁₇H₂₁ClN₂O₄S requires: 384, found: 385[M+H]⁺.

### Example 2y

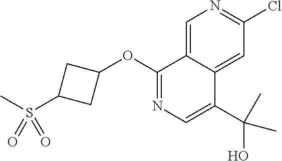 [SMILES: CC(C)(O)c1cnc(OC2CC(S(C)(=O)=O)C2)c2cnc(Cl)cc12]

### Intermediate 54: 2-(6-Chloro-1-((1-(methylsulfonyl)azetidin-3-yl)oxy)-2,7-naphthyridin-4-yl)propan-2-ol

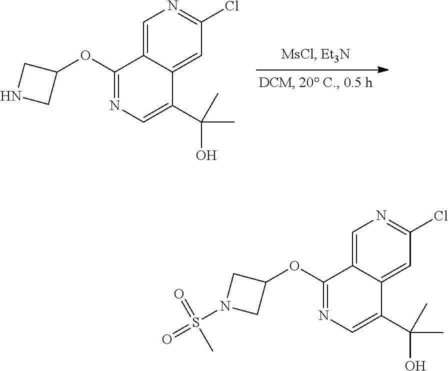 [SMILES: CC(C)(O)c1cnc(OC2CNC2)c2cnc(Cl)cc12 ; CC(C)(O)c1cnc(OC2CN(S(C)(=O)=O)C2)c2cnc(Cl)cc12]

### Step 1: 2-(6-Chloro-1-((1-(methylsulfonyl)azetidin-3-yl)oxy)-2,7-naphthyridin-4-yl)propan-2-ol

To a solution of 2-(1-(azetidin-3-yloxy)-6-chloro-2,7-naphthyridin-4-yl)propan-2-ol (50 mg, 170 umol) and Et₃N (51.7 mg, 511 umol, 71.1 uL) in DCM (5 mL) was added MsCl (14.5 uL, 187 umol). The reaction mixture was stirred at 25° C. for 2 h, then was quenched by addition of water (20 mL) and extracted with EA (20 mL×3). The combined organic layers were dried over with anhydrous sodium sulfate, filtered and concentrated to give a residue. The residue was purified by prep-TLC (PE:EA=0:1) to give the title compound (40 mg, 61% yield) as a white solid.

### Example 2z

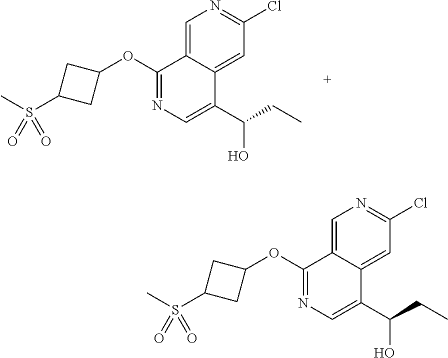

### Intermediates 55 and 56: (S)-1-(6-Chloro-1-((1-(methylsulfonyl)azetidin-3-yl)oxy)-2,7-naphthyridin-4-yl)propan-1-ol and (R)-1-(6-chloro-1-((1-(methylsulfonyl)azetidin-3-yl)oxy)-2,7-naphthyridin-4-yl)propan-1-ol

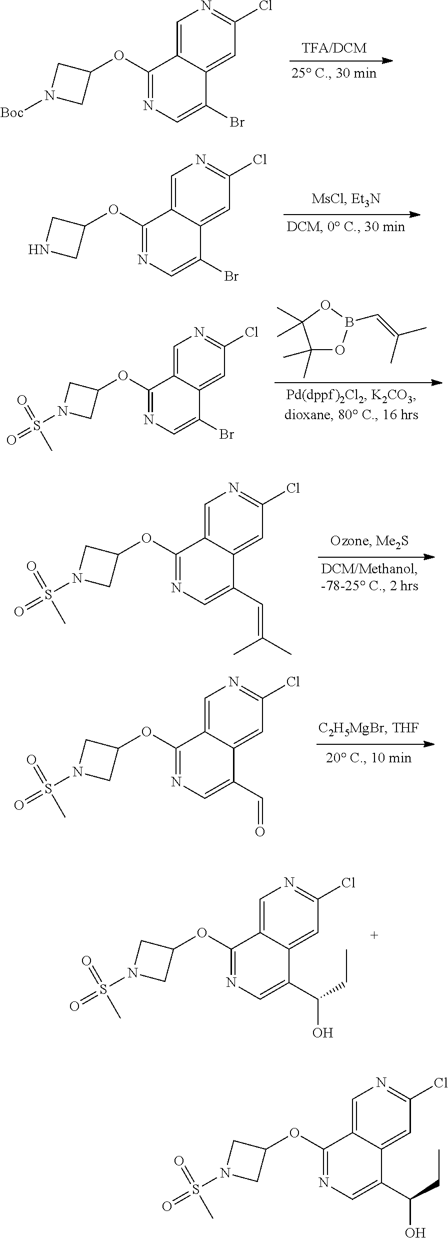

### Step 1: 1-(Azetidin-3-yloxy)-4-bromo-6-chloro-2,7-naphthyridine

To a solution of TFA (16.5 g, 145 mmol) in DCM (11 mL) was added tert-butyl 3-((4-bromo-6-chloro-2,7-naphthyridin-1-yl)oxy)azetidine-1-carboxylate (3.00 g, 7.23 mmol) at 20° C. The reaction mixture was stirred at 20° C. for 30 min, then was concentrated to give the title compound (3.10 g, TFA, crude) as a yellow oil that was used without further purification.

### Step 2: 4-Bromo-6-chloro-1-((1-(methylsulfonyl)azetidin-3-yl)oxy)-2,7-naphthyridine

To a solution of 1-(azetidin-3-yloxy)-4-bromo-6-chloro-2,7-naphthyridine (3.10 g, 7.23 mmol, TFA) in DCM (30 mL) was added Et₃N (1.83 g, 18.1 mmol) at 0° C., followed by a solution of MsCl (911 mg, 7.96 mmol) in DCM (2 mL). Thee reaction mixture was stirred at 0° C. for 1 h, then was quenched with water (10 mL) and extracted with DCM (20 ml×3). The combined organic layers were washed with water (20 mL), dried over anhydrous sodium sulfate, filtered and concentrated to give a residue. The residue was purified by flash silica gel chromatography (Eluent of 0-50% EA/PE) to give the title compound (2.00 g, 67% yield) as an off-white solid.

### Step 3: 6-Chloro-4-(2-methylprop-1-en-1-yl)-1-((1-(methylsulfonyl)azetidin-3-yl)oxy)-2,7-naphthyridine

A mixture of 4-bromo-6-chloro-1-((1-(methylsulfonyl)azetidin-3-yl)oxy)-2,7-naphthyridine (2.00 g, 5.09 mmol), 4,4,5,5-tetramethyl-2-(2-methylprop-1-en-1-yl)-1,3,2-dioxaborolane (974 mg, 5.35 mmol), Pd(dppf)Cl₂ (373 mg, 509 μmol), and K₂CO₃ (1.41 g, 10.2 mmol) in dioxane (40 mL) and water (1 mL) was stirred at 80° C. for 16 h. The reaction mixture was then concentrated, and the residue was purified by flash silica gel chromatography (Eluent of 0-30% EA/DCM) to give the title compound (1.40 g, 75% yield) as an off-white solid.

### Step 4: 6-Chloro-1-((1-(methylsulfonyl)azetidin-3-yl)oxy)-2,7-naphthyridine-4-carbaldehyde

A solution of 6-chloro-4-(2-methylprop-1-en-1-yl)-1-((1-(methylsulfonyl)azetidin-3-yl)oxy)-2,7-naphthyridine (1.20 g, 3.26 mmol) in MeOH (1.2 mL) and DCM (36 ml) was cooled to −78° C. Ozone was bubbled into the reaction mixture for 5 minute under 15 psi. The reaction mixture was then purged with N₂, and then Me₂S (2.03 g, 32.6 mmol) was added into the reaction mixture. The reaction mixture was warmed to 25° C. and stirred for 2 h, then was concentrated to give the title compound (1.10 g, crude) as a white solid. MS (ES+) C₁₃H₁₂ClN₃O₄S requires: 341, found: 342[M+H]⁺.

### Step 5: (S)-1-(6-Chloro-1-((1-(methylsulfonyl)azetidin-3-yl)oxy)-2,7-naphthyridin-4-yl)propan-1-ol and ®-1-(6-chloro-1-((1-(methylsulfonyl)azetidin-3-yl)oxy)-2,7-naphthyridin-4-yl)propan-1-ol

To a solution of 6-chloro-1-((1-(methylsulfonyl)azetidin-3-yl)oxy)-2,7-naphthyridine-4-carbaldehyde (500 mg, 1.46 mmol) in THF (6 ml) was added EtMgBr (3 M, 1.46 ml) at 20° C. in one portion. The reaction mixture was stirred at 20° C. for 10 min, then was quenched with brine (1 mL) and concentrated to give a residue. The residue was purified by flash silica gel chromatography (Eluent of 0-50% EA/PE) to give a racemic mixture of the title compounds (400 mg, 70% yield) as a white solid. ¹H NMR: (400 MHz, CD₃OD): δ ppm 9.45 (s, 1H), 8.15 (s, 1H), 7.90-8.15 (m, 1H), 5.60-5.40 (m, 1H), 5.10-4.90 (m, 1H), 4.50-4.40 (m, 2H), 4.30-4.10 (m, 2H), 2.95 (s, 3H), 2.00-1.90 (m, 2H), 1.10-0.90 (m, 3H). The mixture was separated by chiral SFC (column: Daicel Chiralpak IG (250 mm*30 mm, 10 m); mobile phase: [Neu-MeOH]; B %: 60%-60%) to give the first one of (S)-1-(6-chloro-1-((1-(methylsulfonyl)azetidin-3-yl)oxy)-2,7-naphthyridin-4-yl)propan-1-ol or ®-1-(6-chloro-1-((1-(methylsulfonyl)azetidin-3-yl)oxy)-2,7-naphthyridin-4-yl)propan-1-ol (peak 1, Intermediate 55, 170 mg, 42% yield) and the second one of (S)-1-(6-chloro-1-((1-(methylsulfonyl)azetidin-3-yl)oxy)-2,7-naphthyridin-4-yl)propan-1-ol or ®-1-(6-chloro-1-((1-(methylsulfonyl)azetidin-3-yl)oxy)-2,7-naphthyridin-4-yl)propan-1-ol (peak 2, Intermediate 56, 170 mg, 42% yield) was obtained as a white solid.

### Example 2aa

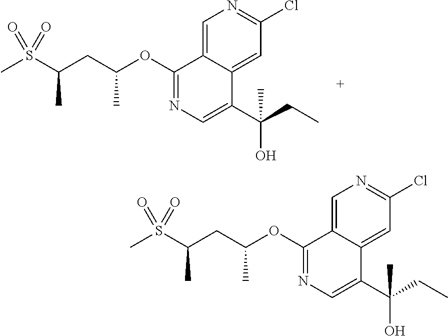

### Intermediates 57 and 58: (R)-2-(6-Chloro-1-(((2R,4R)-4-(methylsulfonyl)pentan-2-yl)oxy)-2,7-naphthyridin-4-yl)butan-2-ol and (S)-2-(6-chloro-1-(((2R,4R)-4-(methylsulfonyl)pentan-2-yl)oxy)-2,7-naphthyridin-4-yl)butan-2-ol

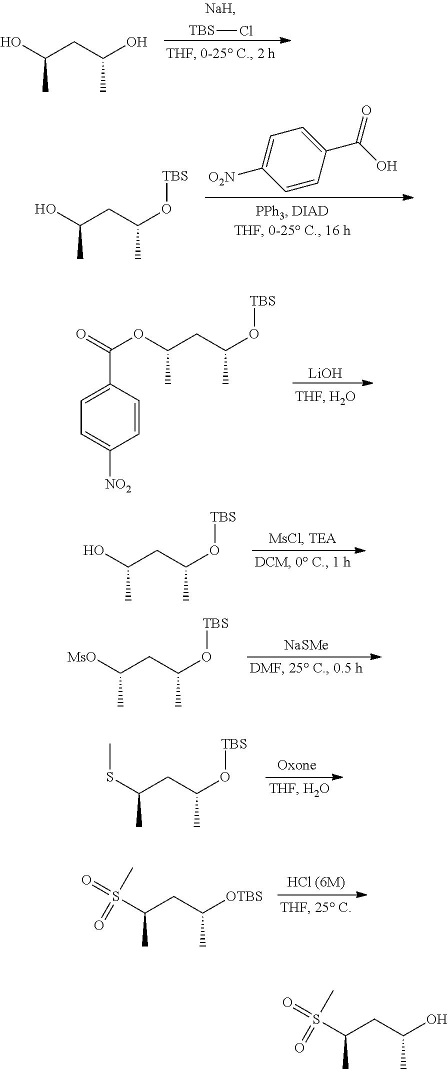

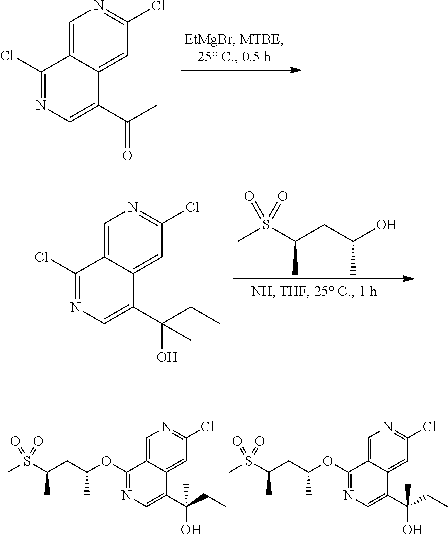

### Step 1: (2R,4R)-4-((tert-Butyldimethylsilyl)oxy)pentan-2-ol

To a solution of (2R,4R)-pentane-2,4-diol (3.80 g, 36.5 mmol) in THF (120 mL) was added NaH (1.75 g, 43.8 mmol, 60% purity) at 0° C. The reaction mixture was stirred for 30 min, then tert-butyldimethylsilyl chloride (6.05 g, 40.1 mmol) was added and the reaction mixture was stirred at 25° C. for 1.5 h. The reaction mixture was then added to water (200 mL) and extracted with EA (150 mL×3). The combined organic layers were dried over anhydrous sodium sulfate, filtered and concentrated to give a residue. The residue was purified by silica gel column chromatography (PE/EA=1:0 to 5:1) to give the title compound (7.80 g, 98% yield) as a colorless oil.

### Step 2: (2S,4R)-4-((tert-Butyldimethylsilyl)oxy)pentan-2-yl 4-nitrobenzoate

To a solution of (2R,4R)-4-((tert-butyldimethylsilyl)oxy)pentan-2-ol (1.00 g, 4.58 mmol), compound 4-nitrobenzoic acid (1.53 g, 9.16 mmol), and triphenylphosphine (3.60 g, 13.7 mmol) in THF (34 mL) was added DIAD (2.78 g, 13.74 mmol) at 0° C. The reaction mixture was stirred at 0° C. for 30 min, then was stirred at 25° C. for 15.5 h, diluted with water (50 mL) and extracted with EA (40 mL×3). The combined organic layers were dried over anhydrous sodium sulfate, filtered and concentrated to give a residue. The residue was purified by silica gel column chromatography (PE/EA=1:0 to 10:1) to give the title compound (1.50 g, 78% yield) as a yellow oil.

### Step 3: (2S,4R)-4-((tert-Butyldimethylsilyl)oxy)pentan-2-ol

To a solution of (2S,4R)-4-((tert-butyldimethylsilyl)oxy)pentan-2-yl 4-nitrobenzoate (1.50 g, 4.08 mmol) in THF (12 mL) and water (4 mL) was added LiOH·H₂O (977 mg, 40.8 mmol). The reaction mixture was stirred at 60° C. for 1 h, then was diluted with water (50 mL) and extracted with EA (40 mL×3). The combined organic layers were dried over anhydrous sodium sulfate, filtered and concentrated to give the title compound (890 mg, 100% yield) as a yellow oil that was used in the next step without further purification.

### Step 4: (2S,4R)-4-((tert-Butyldimethylsilyl)oxy)pentan-2-yl methanesulfonate

To a solution of (2S,4R)-4-((tert-butyldimethylsilyl)oxy)pentan-2-ol (890 mg, 4.07 mmol) in DCM (15 mL) was added TEA (1.24 g, 12.2 mmol) and MsCl (934 mg, 8.15 mmol) at 0° C. The reaction mixture was stirred for 1 h, then was diluted with water (100 mL) and extracted with DCM (80 mL×3). The combined organic layers were dried over anhydrous sodium sulfate, filtered and concentrated to give the title compound (1.20 g, 99% yield) as a yellow oil that was used in the next step without further purification.

### Step 5: tert-Butyldimethyl(((2R,4R)-4-(methylthio)pentan-2-yl)oxy)silane

To a solution of compound (2S,4R)-4-((tert-butyldimethylsilyl)oxy)pentan-2-yl methanesulfonate (1.20 g, 4.05 mmol) in DMF (30 mL) was added sodium methanethiolate (709 mg, 10.1 mmol). The reaction mixture was stirred at 25° C. for 0.5 h, then was diluted with water (80 mL) and extracted with EA (60 mL×3). The combined organic layers were dried over anhydrous sodium sulfate, filtered and concentrated to give the title compound (1.00 g, 99% yield) as a yellow oil that was used in the next step without further purification.

### Step 6: tert-Butyldimethyl(((2R,4R)-4-(methylsulfonyl)pentan-2-yl)oxy)silane

To a solution of tert-butyldimethyl(((2R,4R)-4-(methylthio)pentan-2-yl)oxy)silane (1.00 g, 4.02 mmol) in THF (14 mL) and water (7 mL) was added Oxone® (4.95 g, 8.05 mmol). The reaction mixture was stirred at 25° C. for 0.5 h, then was quenched by addition of saturated aqueous sodium sulfite solution (30 mL) and extracted with EA (20 mL×3). The combined organic layers were dried over anhydrous sodium sulfate, filtered and concentrated to give the title compound (1.10 g, 97% yield) as a yellow oil that was used without further purification.

### Step 7: (2R,4R)-4-(Methylsulfonyl)pentan-2-ol

To a solution of tert-butyldimethyl(((2R,4R)-4-(methylsulfonyl)pentan-2-yl)oxy)silane (1.10 g, 3.92 mmol) in THF (8 mL) was added aqueous HCl (6 M, 2 mL). The reaction mixture was stirred at 25° C. for 0.5 h, then was diluted with water (50 mL) and extracted with EA (40 mL×3). The combined organic layers were dried over anhydrous sodium sulfate, filtered and concentrated to give a residue. The residue was purified by silica gel column chromatography (PE/EA=1:0 to 0:1) to give the title compound (230 mg, 35% yield) as a yellow oil.

### Step 8: 2-(1,6-Dichloro-2,7-naphthyridin-4-yl)butan-2-ol

To a solution of 1-(1,6-dichloro-2,7-naphthyridin-4-yl)ethan-1-one (540 mg, 2.24 mmol) in MTBE (216 mL) was added EtMgBr (3 M, 2.24 mL) at 25° C. The reaction mixture was stirred at 25° C. for 0.5 h, then was quenched by addition of saturated aqueous ammonium chloride solution (20 mL), diluted with water (10 mL) and extracted with EA (40 mL×3). The combined organic layers were dried over anhydrous sodium sulfate, filtered and concentrated to give a residue. The residue was purified by flash silica gel chromatography (Eluent of 0˜40% EA/PE) to give the title compound (320 mg, 50% yield) as a yellow solid.

### Step 9: (R)-2-(6-Chloro-1-(((2R,4R)-4-(methylsulfonyl)pentan-2-yl)oxy)-2,7-naphthyridin-4-yl)butan-2-ol and (S)-2-(6-chloro-1-(((2R,4R)-4-(methylsulfonyl)pentan-2-yl)oxy)-2,7-naphthyridin-4-yl)butan-2-ol

To a solution of (2R,4R)-4-(methylsulfonyl)pentan-2-ol (290 mg, 1.07 mmol) in THF (12 mL) was added NaH (51.3 mg, 1.28 mmol, 60% purity) at 0° C. The cooling bath was removed, and the reaction mixture was stirred at 25° C. for 0.5 h, then 2-(1,6-dichloro-2,7-naphthyridin-4-yl)butan-2-ol (267 mg, 1.60 mmol) was added. The reaction mixture was stirred at 25° C. for 0.5 h, then was quenched with H₂O (30 mL) and extracted with EA (40 mL×3). The combined organic layers were dried over anhydrous sodium sulfate, filtered and concentrated to give a residue. The residue was purified by column chromatography (SiO₂, PE/EA=1/0 to 1/1) to give a mixture of the title compounds (320 mg, 74% yield) as a yellow oil. MS (ES+) C₁₈H₂₅ClN₂O₄S requires: 400, found: 401[M+H]⁺. The mixture was separated by chiral SFC (column: Daicel Chiralpak AD-H (250 mm×30 mm, 5 m); mobile phase: [0.1% NH₃H₂O EtOH]; B %: 30%-30%) to give the first one of (R)-2-(6-chloro-1-(((2R,4R)-4-(methylsulfonyl)pentan-2-yl)oxy)-2,7-naphthyridin-4-yl)butan-2-ol or (S)-2-(6-chloro-1-(((2R,4R)-4-(methylsulfonyl) pentan-2-yl)oxy)-2,7-naphthyridin-4-yl)butan-2-ol (peak 1, Intermediate 57, 130 mg, 41% yield) as a white oil and the second one of (R)-2-(6-chloro-1-(((2R,4R)-4-(methylsulfonyl)pentan-2-yl)oxy)-2,7-naphthyridin-4-yl)butan-2-ol or (S)-2-(6-chloro-1-(((2R,4R)-4-(methylsulfonyl) pentan-2-yl)oxy)-2,7-naphthyridin-4-yl)butan-2-ol (peak 2, Intermediate 58, 130 mg, 41% yield) as a white oil.

### Example 2ab

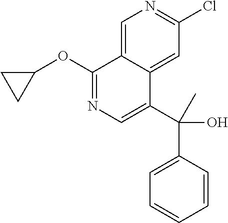 [SMILES: CC(O)(c1ccccc1)c1cnc(OC2CC2)c2cnc(Cl)cc12]

### Intermediate 59: 1-(6-Chloro-1-cyclopropoxy-2,7-naphthyridin-4-yl)-1-phenylethan-1-ol

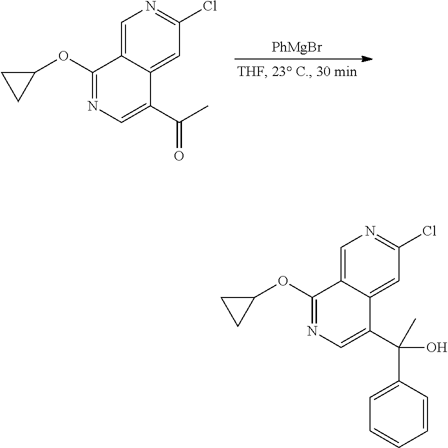 [SMILES: "PhMgBr" ; CC(=O)c1cnc(OC2CC2)c2cnc(Cl)cc12 ; CC(O)(c1ccccc1)c1cnc(OC2CC2)c2cnc(Cl)cc12]

### Step 1: 1-(6-Chloro-1-cyclopropoxy-2,7-naphthyridin-4-yl)-1-phenylethan-1-ol

1-(6-Chloro-1-cyclopropoxy-2,7-naphthyridin-4-yl)ethan-1-one (0.15 g, 0.571 mmol) was stirred in THF (5.71 ml) at 23° C. and phenylmagnesium bromide (1.0 M, 0.685 ml, 0.685 mmol) was added. The reaction mixture was stirred at 23° C. for 30 min, then was diluted with EA, washed with saturated aqueous sodium bicarbonate solution, brine, dried over anhydrous sodium sulfate, filtered and concentrated. The residue was purified by flash-column chromatography on silica gel (20-100% EA:Hexanes) to give the title compound (65 mg, 33%). MS (ES+) C₁₉H₁₇ClN₂O₂ requires: 340, found: 341[M+H]⁺.

### Example 2ac

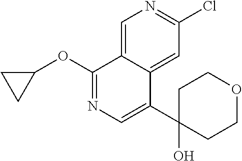 [SMILES: OC1(c2cnc(OC3CC3)c3cnc(Cl)cc23)CCOCC1]

### Intermediate 60: 4-(6-Chloro-1-cyclopropoxy-2,7-naphthyridin-4-yl)tetrahydro-2H-pyran-4-ol

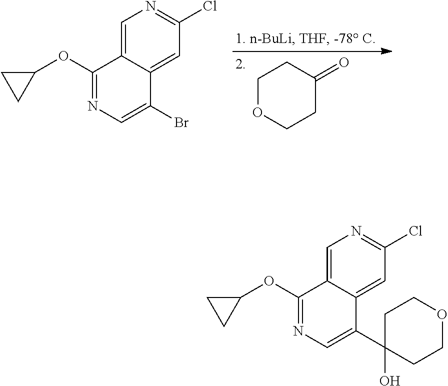 [SMILES: Clc1cc2c(Br)cnc(OC3CC3)c2cn1 ; O=C1CCOCC1 ; OC1(c2cnc(OC3CC3)c3cnc(Cl)cc23)CCOCC1]

### Step 1: −(6-Chloro-1-cyclopropoxy-2,7-naphthyridin-4-yl)tetrahydro-2H-pyran-4-ol

n-Butyllithium (421 μl, 1.052 mmol) was added dropwise to a solution of 4-bromo-6-chloro-1-cyclopropoxy-2,7-naphthyridine (300 mg, 1.001 mmol) in THF (7 mL) at −78° C. The reaction mixture was stirred for 30 min at −78° C., then tetrahydro-4H-pyran-4-one (111 μl, 1.202 mmol) was added dropwise. The reaction mixture was stirred at −78° C. for 40 min, then was quenched by addition of saturated aqueous ammonium chloride solution (4 mL). The cooling bath was removed, and the reaction mixture was diluted with water and extracted with EA. The organic layer was dried over sodium sulfate, filtered, and concentrated. The residue was purified by flash-column chromatography on silica gel (0-90% EA/hexanes) to give the title compound (108 mg, 33.6% yield).

### Example 3a: Synthetic Method 1

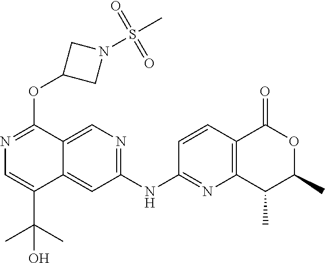 [SMILES: C[C@@H]1OC(=O)c2ccc(Nc3cc4c(C(C)(C)O)cnc(OC5CN(S(C)(=O)=O)C5)c4cn3)nc2[C@H]1C]

### (7S,8R)-2-((5-(2-Hydroxypropan-2-yl)-8-((1-(methylsulfonyl)azetidin-3-yl)oxy)-2,7-naphthyridin-3-yl)amino)-7,8-dimethyl-7,8-dihydro-5H-pyrano[4,3-b]pyridin-5-one (Compound 57)

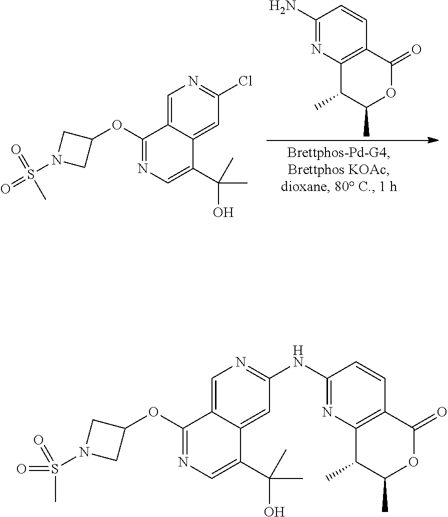 [SMILES: CC(C)(O)c1cnc(OC2CN(S(C)(=O)=O)C2)c2cnc(Cl)cc12 ; C[C@@H]1OC(=O)c2ccc(N)nc2[C@H]1C ; C[C@@H]1OC(=O)c2ccc(Nc3cc4c(C(C)(C)O)cnc(OC5CN(S(C)(=O)=O)C5)c4cn3)nc2[C@H]1C]

### Step 1: (7S,8R)-2-((5-(2-Hydroxypropan-2-yl)-8-((1-(methylsulfonyl)azetidin-3-yl)oxy)-2,7-naphthyridin-3-yl)amino)-7,8-dimethyl-7,8-dihydro-5H-pyrano[4,3-b]pyridin-5-one

To a solution of 2-(6-chloro-1-((1-(methylsulfonyl)azetidin-3-yl)oxy)-2,7-naphthyridin-4-yl)propan-2-ol (30 mg, 80.7 umol) and (7S,8R)-2-amino-7,8-dimethyl-7,8-dihydro-5H-pyrano[4,3-b]pyridin-5-one (17.1 mg, 88.8 umol) in dioxane (2 mL) was added BrettPhos (Pd, G4) (7.43 mg, 8.07 umol), BrettPhos (4.33 mg, 8.07 umol) and potassium acetate (23.8 mg, 242 umol). The mixture was stirred at 80° C. for 1 hour under nitrogen. The reaction mixture was then filtered and concentrated under reduced pressure to give a residue. The reaction mixture was purified by pre-HPLC (column: Phenomenex Synergi C18 150*25*10 um; mobile phase: [water (0.225% FA)—ACN]; B %: 29%-59%, 10 min) to give the title compound (15.4 mg, 30% yield) as a light yellow solid. MS (ES+) C₂₅H₂₉N₅O₆S requires: 527, found: 528[M+H]⁺. ¹H NMR (400 MHz, CD₃OD) δ ppm 9.40 (s, 1H), 9.27 (s, 1H), 8.15 (s, 1H), 8.13 (s, 1H), 7.32 (d, J=8.8 Hz, 1H), 5.60 (s, 1H), 4.70-4.56 (m, 1H), 4.44 (t, J=7.8 Hz, 2H), 4.28-4.11 (m, 2H), 3.33 (s, 6H), 3.05 (s, 4H), 1.82 (s, 6H), 1.57-1.45 (m, 6H).

### Example 4: Inhibition of MAP4K1 Biochemical Enzymatic Activity

MAP4K1 (HPK1) and relevant off-target enzymatic activity was monitored using the Perkin Elmer electrophoretic mobility shift technology platform—the EZReader 2. Fluorescent labeled substrate peptide was incubated in the presence of kinase and ATP, and in the presence of dosed compound, such that each dose of compound resulted in a reflective proportion of the peptide to be phosphorylated. Within the linear, steady-state phase of the kinase enzymatic reaction, the mixed pool of phosphorylated (product) and non-phosphorylated (substrate) peptides was passed through the microfluidic system of the PerkinElmer EZ Reader 2, under an applied electric potential difference. The presence of the phosphate group on the product peptide provided a difference in mass and charge between that of the substrate peptide, resulting in a separation of the substrate and product pools in the sample (Perrin et al. 2010). As the product and substrate peptide mixture passes the lasers within the instrument, these pools are detected (λₑₓ=488 nm, λₑₘ=568 nm) and resolved as separate peaks. The ratio between these peaks reflects the activity of the compound at that concentration, in that well, under those conditions.

### Enzyme Activity Inhibition Assay Protocol:

Inhibitors were dissolved in 100% DMSO at a stock concentration of 10 mM. A 100×, 10-point, 4-fold serial dilution of each inhibitor was created in 100% DMSO either manually or on a Hamilton STAR liquid handler, starting at a relevant concentration, usually 1 mM. A volume of 0.130 μL of each concentration was transferred to the relevant wells of a 384-well plate (Greiner 781 201) in duplicate using a TTPLabtech Mosquito nano-litre dispenser. Using a Multidrop Combi, the remaining constituents of the kinase reaction were added to the 130 nL of dosed compound as follows (see table below for final reaction details):

Enzyme activity assays at the ^{APP}KM for ATP or 1 mM ATP: In each well of a 384-well plate, 0.1-15 nM of untreated enzyme was incubated in a total of 13 μL of buffer (100 mM HEPES pH 7.5, 0.015% Brij 35, 10 mM MgCl₂, 1 mM DTT) with 1.5 μM fluorescent peptide and 20-1000 μM ATP, at 25° C., for 60-180 minutes in the presence or absence of a dosed concentration series of compound (1% DMSO final concentration). The kinase reactions were stopped by the addition of 70 μl of Stop buffer (100 mM HEPES pH 7.5, 0.015% Brij 35, 35 mM EDTA and 0.2% of Coating Reagent 3 (Caliper Lifesciences)). The plates were read on a Caliper EZReader 2 as described above.

TABLE 2
Kinase Reaction Conditions

| | | | Substrate | Kinase |
|---|---|---|---|---|
| Enzyme | Enzyme | ATP | Peptide & | Reaction |
| (Source) | Concentration | Concentration | Concentration | Time |
| HPK1 | 0.5 nM | 26 μM | S6K2tide, 1.5 μM | 120 min. |
| (Invitrogen) | | | | |
| HPK1 | 0.25 nM | 1000 μM | S6K2tide, 1.5 μM | 120 min. |
| (Invitrogen) | | | | |
| LCK | 12 nM | 26 μM | FL4tide, 1.5 μM | 60 min. |
| (Invitrogen) | | | | |
| HGK | 0.1 nM | 50 μM | FL25tide, 1.5 μM | 60 min. |
| (Invitrogen) | | | | |
| GLK | 15 nM | 20 μM | PKAtide, 2 μM | 180 min. |
| (SignalChem) | | | | |

S6K2tide; Carna Biosciences (5-FAM-Proprietary Sequence-CONH2)
FL4tide; Perkin Elmer (5-FAM-EGIYGVLFKKK (SEQ ID NO: 1)-CONH₂)
FL25tide; Perkin Elmer (5-FAM-VDGKEIYNTIRRK (SEQ ID NO: 2)-CONH₂)
PKAtide; Anaspec Peptide Co.(5-FAM-GRTGRRNSI (SEQ ID NO: 3)-CONH₂)

Perrin D, Frémaux C, Shutes A. Capillary microfluidic electrophoretic mobility shift assays: application to enzymatic assays in drug discovery. Expert Opin Drug Discov. 2010, 5(1):51-63.

The results obtained in these experiments for compounds prepared according to the examples are summarized in Table 3 below.

TABLE 3

| Compound | MAP4K1 | IL-2 Primary T |
|---|---|---|
| No. | IC50 (nM) | Cell EC50 (nM) |
| 1 | 121.00 | |
| 2 | 64.30 | |
| 3 | 23.10 | |
| 4 | 2.34 | |
| 5 | 18.40 | |
| 6 | 9.23 | |
| 7 | 17.50 | |
| 8 | 4.73 | |
| 9 | 1.63 | |
| 10 | 1.19 | |
| 11 | 15.30 | |
| 12 | 46.50 | |
| 13 | 6.36 | |
| 14 | 0.25 | |
| 15 | 3.06 | |
| 16 | 0.61 | |
| 17 | 0.30 | 10 |
| 18 | 1.65 | |
| 19 | 14.30 | |
| 20 | 3.48 | |
| 21 | 1.98 | |
| 22 | 23.60 | |
| 23 | 3.34 | |
| 24 | 0.39 | |
| 25 | 27.00 | |
| 26 | 1.06 | |
| 27 | 1.70 | 42 |
| 28 | 3.03 | |
| 29 | 1.43 | |
| 30 | 2.38 | |
| 31 | 0.42 | 47 |
| 32 | 15.50 | |
| 33 | 0.56 | |
| 34 | 2.53 | 132 |
| 35 | 67.30 | |
| 36 | 3.86 | |
| 37 | 2.01 | |
| 38 | 11.10 | 273 |
| 39 | 0.46 | 47 |
| 40 | 2.09 | 169 |
| 41 | 6.55 | |
| 42 | 3.38 | |
| 43 | 6.72 | |
| 44 | 1.53 | |
| 45 | 1.91 | |
| 46 | 31.90 | |
| 47 | 1.57 | |
| 48 | 39.50 | |
| 49 | 0.53 | |
| 50 | 4.51 | |
| 51 | 12.30 | |
| 52 | 23.20 | |
| 53 | 3.75 | |
| 54 | 16.90 | |
| 55 | 15.90 | |
| 56 | 0.45 | |
| 57 | 0.20 | |
| 58 | 2.52 | |
| 59 | 12.90 | |
| 60 | 0.29 | 65 |
| 61 | 0.39 | 23 |
| 62 | 1.66 | |
| 63 | 2.94 | |
| 64 | 0.67 | |
| 65 | 0.39 | |
| 66 | 1.74 | |
| 67 | 0.66 | |
| 68 | 0.41 | |
| 69 | 0.89 | |
| 70 | 0.49 | |

Additional Compounds 71-74 falling within the scope of Formula (I), see Table 4 below, were also tested in the MAP4K1 (HPK1) enzymatic assay of Example 4 and all of these compounds had inhibitory activities of greater than 10 micromolar.

TABLE 4

| Cpd. No. | Structure |
|---|---|
| 71 | 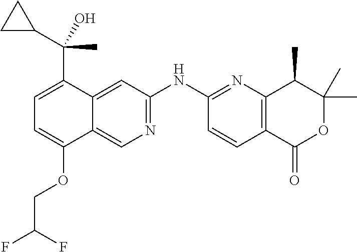 [SMILES: C[C@@H]1c2nc(Nc3cc4c([C@@](C)(O)C5CC5)ccc(OCC(F)F)c4cn3)ccc2C(=O)OC1(C)C] |
| 72 | 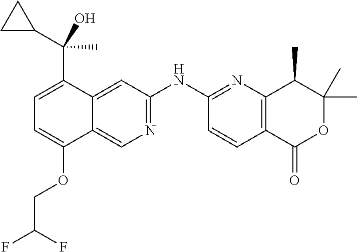 [SMILES: C[C@@H]1c2nc(Nc3cc4c([C@](C)(O)C5CC5)ccc(OCC(F)F)c4cn3)ccc2C(=O)OC1(C)C] |
| 73 | 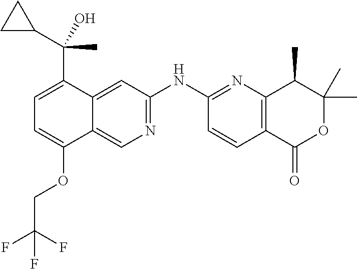 [SMILES: C[C@@H]1c2nc(Nc3cc4c([C@@](C)(O)C5CC5)ccc(OCC(F)(F)F)c4cn3)ccc2C(=O)OC1(C)C] |
| 74 | 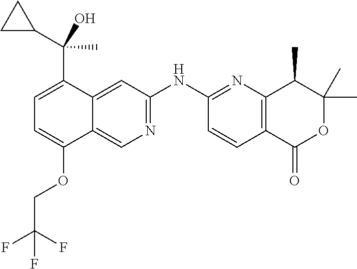 [SMILES: C[C@@H]1c2nc(Nc3cc4c([C@](C)(O)C5CC5)ccc(OCC(F)(F)F)c4cn3)ccc2C(=O)OC1(C)C] |

### Example 5: T Cell Enhancement of Cytokines

Isolation and Expansion of T Cells from Whole Blood

T cells are isolated from whole blood of healthy donors by immunomagnetic negative selection following manufacture's protocol (StemCell Technologies, human T cell isolation kit). Purity of isolated cells is assessed by flow cytometry and yields 95-98% CD3⁺ T cells. For expansion of T cells, 1×10⁶ cells/well are plated in serum free cell expansion media (ThermoFisher) containing 30U of recombinant human IL2 (R&D) and stimulated with 25 ul of CD3/CD28 beads (Invitrogen) in 24 well plates for 3-4 days. T cells are then expanded in 175 cm flasks and maintained at a cell density of 1 to 2.5×10⁶ cells/ml days by addition of ⅔ of fresh media every 2-3 days. After 10-14 days, cells are frozen in BamBanker freezing media (Thermo) and stored in liquid nitrogen. Phenotypic analysis of expanded T cells by flow cytometry, routinely shows 60% cells are CD8⁺ T cells upon freezing.

### Cytokine Measurement

For IL2 measurement, expanded CD3⁺ T cells are dispensed at 100K cells/well (cultured in X-VIVO 10 Serum-free media) and are stimulated with plate-bound anti-CD3 and soluble anti-CD28 in the presence of vehicle or compound of the disclosure at various concentrations for 24 h. As outlined in the manufacturer's protocol (Cisbio), 16 μL of conditioned media is transferred to a white 384-well low volume plate. Following a 24 h incubation with the anti-IL2 antibodies, the homogenous time resolved fluorescence (HTRF) is measured.

### Example 6: Inhibition of Anti-Tumor Activity in a Syngeneic Mouse Model

Generation of the MCA205 Syngeneic xenograft anti-tumor efficacy study Six to eight-week-old female, C57BL/6 mice (Jackson Labs, Bar Harbor, ME) are implanted subcutaneously on the left flank with 1×10⁶ MCA205 cells/mouse. After tumors reach an average volume of 50 mm³, mice are randomized into treatment groups, 10 mice per group, with tumors in the size range of 30-70 mm³. Compounds of the disclosure 10-30 mg/kg, anti-mouse PD-L1 mAb (B7 H1, clone #10F.9G2 Bio-X-cell, Lebanon, NH) and vehicle either alone or in different combinations are administered to tumor bearing mice. Reduction in tumor volume is measured [mm³] over time.

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

What is claimed is:

1. A compound of formula I: 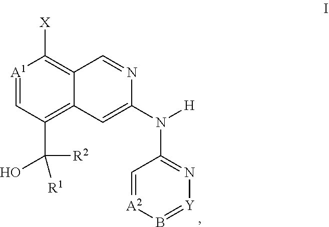
or a pharmaceutically acceptable salt thereof,
wherein:
A¹ is selected from N and CH;
A² is selected from CH and N;
X is selected from C₁₋₃ alkyl, OR³, NHR⁴ and halogen;
B is selected from CR⁵ and N, Y is CR⁶, or Y and B, taken together, form a 5 to 7-membered heterocycle or C₅₋₆ cycloalkyl, wherein said heterocycle or cycloalkyl is optionally substituted with 1-6 R⁷;
R¹ and R² are each independently selected from hydrogen, C₁₋₆ alkyl, C₁₋₆ haloalkyl, C₁₋₆ alkyl substituted with OR⁸, phenyl, C₃₋₆ cycloalkyl and 4 to 6-membered heterocycle, or
R¹ and R², taken together with the atoms to which they are attached, form a C₃₋₆ cycloalkyl or 4 to 6-membered heterocycle;
R³ is selected from C₁₋₃ alkyl, C₃₋₆ cycloalkyl and 4 to 6-membered heterocycle, wherein said alkyl, cycloalkyl, and heterocycle are optionally substituted with 1-3 R⁹;
R⁴ is selected from hydrogen, C₁₋₃ alkyl, C₃₋₅ cycloalkyl and 4 to 6-membered heterocycle, wherein said alkyl, cycloalkyl, and heterocycle are optionally substituted with 1-3 R¹⁰;
R⁵ is selected from hydrogen, COOH, CN, halogen, and C₁₋₃ alkoxy;
R⁶ is selected from C₁_₅ alkyl, C₄₋₆ cycloalkyl, 3 to 6-membered heterocycle, NHR¹¹, NR¹²R¹³ and OR¹⁴, wherein said alkyl, cycloalkyl or heterocycle is optionally substituted with OH, NH₂, 1-4 halogen or R¹⁵;
each R⁷ is independently selected from C₁₋₃ alkyl, halogen and OH, wherein said alkyl is optionally substituted with 1-3 halogen, or
two R⁷ attached to the same carbon form an oxo, or
two R⁷ attached to the same carbon atom taken together with the carbon atom to which they are attached form a C₃₋₅ cycloalkyl;
R⁸ is H or C₁₋₃ alkyl;
each R⁹ is independently selected from C₁₋₃ alkyl, C₃₋₆ cycloalkyl substituted with halogen, halogen, C(O)Me, SO₂Me, C(O)NR¹⁶R¹⁷, C₁₋₃ alkoxy, and OH;
each R¹⁰ is independently selected from C₁₋₃ alkyl, C₃₋₆ cycloalkyl substituted with halogen, halogen, SO₂Me, C(O)NR¹⁶R¹⁷, C₁₋₃ alkoxy, and OH;
R¹¹ is selected from C₁₋₆ alkyl and C₃₋₆ cycloalkyl, wherein said alkyl or cycloalkyl is optionally substituted with 1-3 R¹⁸;
R¹² and R¹³ are each is independently selected from C₁₋₆ alkyl and C₃₋₆ cycloalkyl, wherein said alkyl or cycloalkyl is optionally substituted with 1-3 R¹⁸;
R¹⁴ is selected from C₁₋₆ alkyl and C₃₋₆ cycloalkyl, wherein said alkyl or cycloalkyl is optionally substituted with 1-3 R¹⁸;
R¹⁵ is OH, C₁₋₃ alkyl or C₃₋₅ cycloalkyl;
R¹⁶ and R¹⁷ are each is independently selected from C₁₋₆ alkyl and C₃₋₆ cycloalkyl, wherein said alkyl or cycloalkyl is optionally substituted with 1-3 R¹⁹;
each R¹⁸ is independently halogen; and
each R¹⁹ is independently halogen.

2. The compound of claim 1, wherein the compound is represented by Formula II: 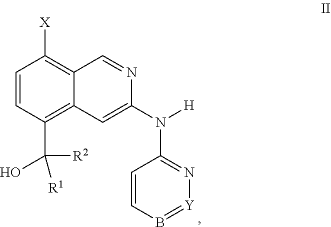
or a pharmaceutically acceptable salt thereof.

3. The compound of claim 1, wherein the compound is represented by Formula III: 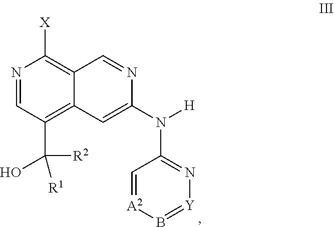
or a pharmaceutically acceptable salt thereof.

4. The compound of claim 3, wherein the compound is represented by Formula IV(A) or IV(B): 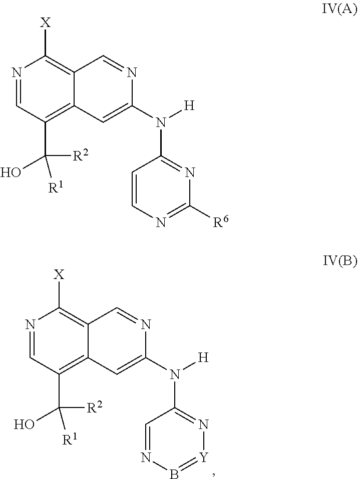
or a pharmaceutically acceptable salt thereof.

5. The compound of claim 3, wherein the compound is represented by Formula V: 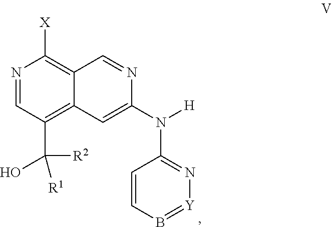
or a pharmaceutically acceptable salt thereof,
wherein Y and B, taken together, form a 5 to 7-membered heterocycle or C₅₋₆ cycloalkyl, and said heterocycle or cycloalkyl is optionally substituted with 1-6 R⁷.

6. The compound of claim 4, wherein the compound is represented by Formula VI: 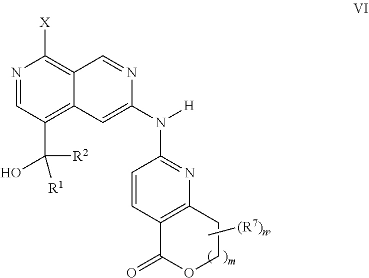
or a pharmaceutically acceptable salt thereof, wherein:
each R⁷ is independently selected from C₁₋₃ alkyl, halogen and OH, wherein said alkyl is optionally substituted with 1-3 halogen, or
two R⁷ attached to the same carbon atom taken together with the carbon atom to which they are attached form a C₃₋₅ cycloalkyl;
n is 0, 1, 2, 3, 4, 5 or 6; and
m is 0, 1 or 2.

7. The compound of claim 6, wherein the compound is represented by Formula VII: 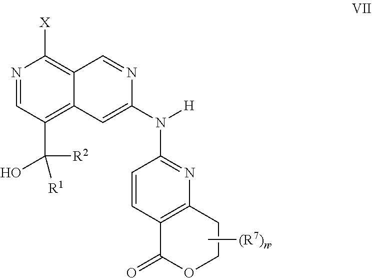
or a pharmaceutically acceptable salt thereof,
wherein n is 0, 1, 2, 3 or 4.

8. The compounds of any one of claims 1-7, or a pharmaceutically acceptable salt thereof, wherein:
X is NHR⁴; and
R⁴ is CH₃ or cyclopropyl.

9. The compounds of any one of claims 1-7, or a pharmaceutically acceptable salt thereof, wherein X is OR³.

10. The compounds of any one of claims 1-9, or a pharmaceutically acceptable salt thereof, wherein:
R¹ and R² are each independently selected from hydrogen, C₁₋₂ alkyl, C₁₋₂ haloalkyl, C₁₋₃ alkyl substituted with OR⁸, phenyl and C₃₋₄ cycloalkyl, or
R¹ and R², taken together with the atoms to which they are attached, form a 4 to 6-membered heterocycle; and
R⁸ is C₁₋₂ alkyl.

11. The compounds of any one of claims 1-9, or a pharmaceutically acceptable salt thereof, wherein:
R¹ and R² are each independently selected from hydrogen, CH₃, CH₂CH₃, CH₂OCH₃, CHF₂, CF₃, cyclobutyl, cyclopropyl and phenyl, or
R¹ and R², taken together with the atoms to which they are attached, form tetrahydropyran.

12. The compounds of any one of claims 1-9, or a pharmaceutically acceptable salt thereof, wherein:
R¹ and R² are each independently selected from hydrogen, CH₃, CH₂CH₃, CH₂OCH₃, CHF₂, CF₃, cyclobutyl, cyclopropyl and phenyl, or
R¹ and R², taken together with the atoms to which they are attached, form

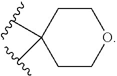 [SMILES: *C1(*)CCOCC1]

13. The compounds of any one of claims 1-7 and 9-12, or a pharmaceutically acceptable salt thereof, wherein:
R³ is selected from C₁₋₃ alkyl, C₃₋₄ cycloalkyl and 4-membered heterocycle; wherein said alkyl, cycloalkyl, and heterocycle are optionally substituted with 1-3 R⁹; and
each R⁹ is independently selected from C₁₋₃ alkyl, halogen, C(O)Me and SO₂Me.

14. The compound of any one of claims 1-7 and 9-12, or a pharmaceutically acceptable salt thereof, wherein:
R³ is 4-membered heterocycle containing nitrogen;
R⁹ is C(O)Me; and
a ring nitrogen of the 4-membered heterocycle is bonded to —C(O)Me.

15. The compounds of any one of claims 1-7 and 9-12, or a pharmaceutically acceptable salt thereof, wherein:
R³ is selected from CH₃, CH₂CH₃, CH₂CH₂CH₃, cyclopropyl, cyclobutyl, azetidinyl, each optionally substituted with 1-3 R⁹; and
each R⁹ is independently selected from CH₃, F, C(O)Me and SO₂Me.

16. The compounds of anyone of claims 1-7 and 9-12, or a pharmaceutically acceptable salt thereof, wherein R³ is selected from CH₃, CH₂CH₃, CH₂CHF₂, CH₂CF₃, CH₂CH₂CH₃, cyclopropyl:

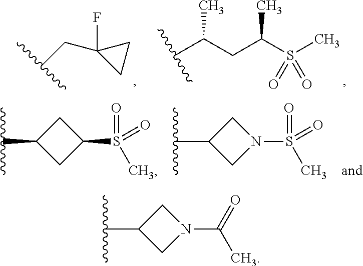

17. The compounds of any one of claims 1-4 and 8-15, or a pharmaceutically acceptable salt thereof, wherein R⁵ is CN.

18. The compounds of any one of claims 1-4 and 8-16, or a pharmaceutically acceptable salt thereof, wherein:
R⁶ is selected from C₁₋₄ alkyl, and 4 to 5-membered heterocycle, wherein said alkyl or heterocycle is optionally substituted with 1-4 halogen or R¹⁵; and
R¹⁵ is C₁₋₂ alkyl.

19. The compounds of any one of claims 1-4 and 8-16, or a pharmaceutically acceptable salt thereof, wherein R⁶ is selected from CH(CH₃)₂, CF(CH₃)₂, C(CH₃)₃,

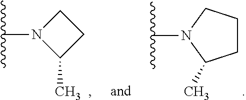

20. The compounds of any one of claims 1-16, or a pharmaceutically acceptable salt thereof, wherein:
each R⁷ is independently CH₃, or
two R⁷ attached to the same carbon form an oxo, or
two R⁷ attached to the same carbon atom taken together with the carbon atom to which they are attached form cyclopropyl.

21. The compounds of any one of claims 1-16, or a pharmaceutically acceptable salt thereof, wherein Y and B, taken together, form: 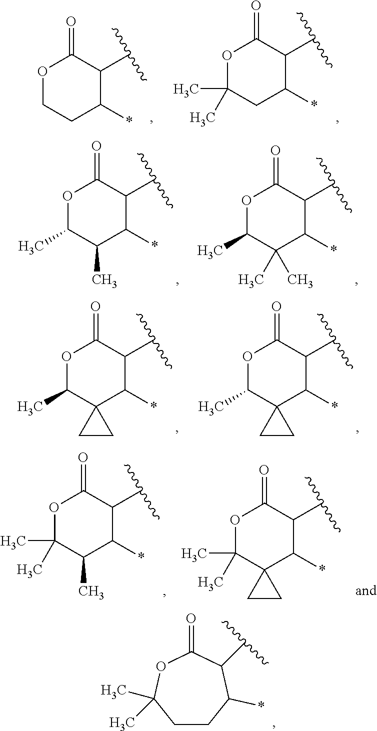
wherein [symbol] represents a bond to A², and [symbol] represents a bond to N.

22. A compound of Table 1 or a pharmaceutically acceptable salt thereof.

23. A pharmaceutical composition comprising the compound of any one of claims 1-22, or a pharmaceutically acceptable salt thereof, and a pharmaceutically acceptable carrier or excipient.

24. A method of inhibiting MAP4K1 in a subject in need thereof, comprising contacting MAP4K1 with an effective amount of the compound of any one of claims 1-22 or a pharmaceutically acceptable salt thereof; or the pharmaceutical composition of claim 23.

25. A method for enhancing an immune response in a subject in need thereof, comprising administering to said subject an effective amount of the compound of any one of claims 1-22 or a pharmaceutically acceptable salt thereof; or the pharmaceutical composition of claim 23.

26. A method for treating a MAP4K1-dependent disorder or disease in a subject in need thereof, comprising administering to said subject an effective amount of the compound of any one of claims 1-22 or a pharmaceutically acceptable salt thereof; or the pharmaceutical composition of claim 23.

27. The method of claim 26, wherein said MAP4K1-dependent disease or disorder is a cancer.

28. The method of claim 27, wherein the cancer comprises at least one cancer selected from the group consisting of colon cancer, pancreatic cancer, breast cancer, prostate cancer, lung cancer, ovarian cancer, cervical cancer, renal cancer, bladder cancer, stomach cancer, liver cancer, cancer of the head and neck, lymphoma, leukemia, and melanoma.

29. The method of claim 26 or 27, wherein said method further comprises administering an additional anti-cancer agent to said subject.

30. The method of claim 26, wherein the MAP4K1-dependent disorder or disease is a viral infection.

31. A compound of any one of claims 1-22, or a pharmaceutically acceptable salt thereof, for use in the treatment of a MAP4K1-dependent disease or disorder.

32. A compound for use according to claim 31, wherein the MAP4K1-dependent disease or disorder is a cancer.

33. The use of a compound of any one of claims 1-22, or a pharmaceutically acceptable salt thereof, for the preparation of a medicament for the treatment a MAP4K1-dependent disease or disorder.


SEQUENCE LISTING (3 sequences)

SEQ ID NO: 1 - protein, 11 aa, synthetic construct
  REGION 1..11: note = Synthetic peptide
EGIYGVLFKKK

SEQ ID NO: 2 - protein, 13 aa, synthetic construct
  REGION 1..13: note = Synthetic peptide
VDGKEIYNTIRRK

SEQ ID NO: 3 - protein, 9 aa, synthetic construct
  REGION 1..9: note = Synthetic peptide
GRTGRRNSI